(19) United States
(12) Patent Application Publication
(10) Pub. No.: US 2022/0409652 A1
(43) Pub. Date: Dec. 29, 2022

(54) miRNA-BASED PHARMACEUTICAL COMPOSITIONS AND USES THEREOF FOR THE PREVENTION AND THE TREATMENT OF TISSUE DISORDERS

(71) Applicant: NOVADIP BIOSCIENCES, Mont-Saint-Guibert (BE)

(72) Inventor: DENIS DUFRANE, LASNE (BE)

(73) Assignee: NOVADIP BIOSCIENCES, Mont-Saint-Guibert (BE)

(21) Appl. No.: 17/781,150

(22) PCT Filed: Nov. 27, 2020
(86) PCT No.: PCT/EP2020/083705

§ 371 Date: May 31, 2022

(30) Foreign Application Priority Data
Nov. 29, 2019 (EP) 19212642.3
Nov. 29, 2019 (EP) 19212678.7

(51) Int. Cl. A61K 31/7088; A61P 19/08; A61P 19/04

(52) U.S. Cl. CPC A61K 31/7088; A61P 19/08; A61P 19/04

(57) ABSTRACT

The present invention relates to a pharmaceutical composition comprising a therapeutically effective amount of (i) at least three miRNAs selected in any one of Table 1, Table 2, Table 3, Table 4, Table 5, Table 6, Table 7, Table 8, Table 9, Table 10, Table 11 or Table 12 and (ii) a pharmaceutical acceptable vehicle. The pharmaceutical composition according to the invention may be of therapeutic use for the prevention and/or the treatment of tissue disorders, including, but not limited to skin disorders, bone disorders and/or cartilage disorders.

### FIELD OF INVENTION

The present invention relates to the prevention and the treatment of tissue disorders, including skin disorders, bone disorders and cartilage disorders. More particularly, the invention relates to pharmaceutical compositions comprising a cocktail of RNAs, in particular miRNAs, that possesses tissue regenerating and/or repairing properties, including osteogenic and/or chondrogenic properties.

### BACKGROUND OF INVENTION Tissue reconstruction encompasses bone and cartilage reconstruction, but also skin (including dermis and epidermis) and muscle reconstruction.

Bone defect is a lack of bone tissue in a body area, where bone should normally be. Bone defects can be treated by various surgical methods. Surgical methods of bone defect reconstruction include inter alia decortication, excision and fixation, cancellous bone grafting and the Ilizarov intercalary bone transport method. However, often there are factors that impair bone healing, like diabetes mellitus, immunosuppressive therapy, poor locomotor status and others that one has to take into account when a procedure is planned. In addition, patients commonly have prolonged ambulatory impairment with suboptimal functional and aesthetic results.

Tissue engineering involves the restoration of tissue structure and/or function through the use of living cells. The general process consists of cell isolation and proliferation, followed by a re-implantation procedure in which a scaffold material is used. Mesenchymal stem cells (MSCs) provide a good alternative to cells from mature tissue and have a number of advantages as a cell source for tissue regeneration, including skin, bone and/or cartilage tissue regeneration.

By definition, a stem cell is characterized by its ability to undergo self-renewal and its ability to undergo multilineage differentiation and form terminally differentiated cells. Ideally, a stem cell for regenerative medicinal applications should meet the following set of criteria: (i) should be found in abundant quantities (millions to billions of cells); (ii) can be collected and harvested by a minimally invasive procedure; (iii) can be differentiated along multiple cell lineage pathways in a reproducible manner; (iv) can be safely and effectively transplanted to either an autologous or allogeneic host.

Studies have demonstrated that stem cells have the capacity to differentiate into cells of mesodermal, endodermal and ectodermal origins. The plasticity of MSCs most often refers to the inherent ability retained within stem cells to cross lineage barriers and to adopt the phenotypic, biochemical and functional properties of cells unique to other tissues. Adult mesenchymal stem cells can be isolated from bone marrow and adipose tissue, for example.

Adipose tissue-derived stem cells are multipotent and have profound regenerative capacities. Osteogenic differentiated ASCs were shown to have a great healing potential in various pre-clinical models when seeded on various scaffolds, such as β-tricalcium phosphate (β-TCP), hydroxyapatite (HA), type I collagen, poly-lactic-co-glycolic acid (PLGA) and alginate. The international patent application WO2013/059089 relates to a bone paste comprising stem cells and a mixture of calcium phosphate cement such as tricalcium phosphate and hydroxyapatite. US2011/104230 discloses a bone patch comprising scaffold material comprising synthetic ceramic material, mesenchymal stem cells and signaling molecules.

However, despite encouraging results in small animal models, critical size bone reconstruction using ASCs loaded on scaffolds remains limited by the large size of bone defect and consequently by the size of the implant to engineer. The cellular engraftment of the seeded cells is also limited by the poor diffusion of oxygen and nutrients. In addition, the cellular position within the scaffold is a major limitation for their in vitro and in vivo survival. Bioreactors with flow perfusion of scaffolds were designed to improve cell migration within the implant for a more homogenous cellular distribution, cell survival by delivering oxygen and nutrients to the core of the implant, and osteogenic cell differentiation (by the fluid shear force). Although these techniques are promising, relevant pre- and clinical data in large animal models are limited.

Recently, the publication WO2019/057862 disclosed a biomaterial having a multi-dimensional structure comprising osteogenic differentiated adipose tissue-derived stem cells (ASCs), a ceramic material and an extracellular matrix, wherein the biomaterial secretes osteoprotegerin (OPG) and comprises insulin-like growth factor (IGF1) and stromal cell-derived factor 1-alpha (SDF-1α).

In addition, the publication WO2020/058511 described a biomaterial having a multi-dimensional structure comprising differentiated adipose tissue-derived stem cells (ASCs), an extracellular matrix and gelatin. It was shown that said biomaterial may be used for treating tissue defect, such as bone, cartilage or skin defects.

Whereas such biomaterials may be suitable for autologous graft, on the other hand, allogenic or xenogeneic grafts cannot be however performed, because they may elicit an immune response hereby resulting in the rejection of the graft or they may carry adventitious pathogens resulting in infection of the recipient with the biomaterial.

Sterilization processes are often performed to alleviate these issues. However, these harsh conditions often deteriorate the biological properties of the sterilized material.

There is thus still a need in the art for tissue engineered materials for tissue reconstruction and/or regeneration that are fully biocompatible and provide appropriate mechanical features for the designated applications, although usable on a broad range of tissues.

There is also a need to provide biomaterials for tissue reconstruction and/or regeneration that are adapted to allogenic or xenogeneic grafts. Finally, there is also a need to provide a sterile biomaterial that maintains the biological properties as compared to the fresh biomaterial, i.e. a biomaterial prior of being sterilized.

There is a need to provide the necessary ingredients for treating tissue disorders, including bone or cartilage or skin disorders that would be safely administered and that would not elicit a significant immune reaction in the recipient individual.

### SUMMARY

A first aspect of the invention relates to a pharmaceutical composition comprising (i) a therapeutically effective amount of at least three miRNAs selected in any one of Table 1, Table 2, Table 3, Table 4, Table 5, Table 6, Table 7, Table 8, Table 9, Table 10, Table 11 or Table 12, and (ii) a pharmaceutical acceptable vehicle.

In some embodiments, said at least three miRNAs are selected in a group comprising hsa-miR-210-3p, hsa-miR-409-3p, hsa-let-7i-5p, hsa-miR-24-3p, hsa-miR-382-5p, hsa-miR-4485-3p, and a combination thereof. In certain embodiments, said at least three miRNAs are selected in a group comprising hsa-miR210-3p, hsa-miR-409-3p, hsa-miR-4454, hsa-miR-619-5p, hsa-miR-3607-5p, hsa-miR-3613-3p, hsa-miR-664b-5p, hsa-miR-3687, hsa-miR-3653-5p, hsa-miR-664b-3p, and a combination thereof. In some embodiments, said at least three miRNAs comprise hsa-miR210-3p and/or hsa-miR-409-3p. In certain embodiments, the composition is desiccated and/or sterilized.

Another aspect of the invention pertains to a pharmaceutical composition according to the instant disclosure, for use as a medicament. In some embodiments, the pharmaceutical composition is for use for the prevention and/or the treatment of a tissue disorder. In certain embodiments, said tissue is selected from the group comprising bone tissue, cartilage tissue, skin tissue, muscular tissue, epithelial tissue, endothelial tissue, connective tissue, neural tissue and adipose tissue. In some embodiments, the pharmaceutical composition is for use for the prevention and/or the treatment of a bone disorder and/or a cartilage disorder. In certain embodiments, the pharmaceutical composition is for use for the prevention and/or the treatment of a skin disorder. In some embodiments, the tissue disorder is selected in a group comprising aplasia cutis congenita; a burn; a cancer, including a breast cancer, a skin cancer and a bone cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer; arthritis; bone fracture; bone frailty; Caffey's disease; congenital pseudarthrosis; cranial deformation; cranial malformation; delayed union; infiltrative disorders of bone; hyperostosis; loss of bone mineral density; metabolic bone loss; osteogenesis imperfecta; osteomalacia; osteonecrosis; osteopenia; osteoporosis; Paget's disease; pseudarthrosis; sclerotic lesions; spina bifida; spondylolisthesis; spondylolysis; chondrodysplasia; costochondritis; enchondroma; hallux rigidus; hip labral tear; osteochondritis dissecans; osteochondrodysplasia; polychondritis; and the likes. In certain embodiments, the pharmaceutical composition is for use for tissue reconstruction.

In a still other aspect, the invention relates to a method for producing a composition comprising at least three miRNAs selected in any one of Table 1, Table 2, Table 3, Table 4, Table 5, Table 6, Table 7, Table 8, Table 9, Table 10, Table 11 or Table 12, said method comprising the steps of
1) culturing a combination comprising (i) viable cells capable to undergo tissue differentiation, and (ii) a particulate material so as to obtain a multidimensional structure comprising an extracellular matrix secreted by said cells, wherein the said cells have tissue regeneration and/or tissue repairing properties, wherein the cells and the particulate material are embedded in the extracellular matrix, and wherein said multidimensional structure comprises a RNA content comprising at least three miRNAs selected in selected in Table 1, Table 2, Table 3, Table 4, Table 5, Table 6, Table 7, Table 8, Table 9, Table 10, Table 11 or Table 12;
2) extracting the RNAs content produced in step 1), in particular the miRNAs content.

In some embodiments, the miRNAs content includes cellular miRNAs and/or exosomes-derived miRNAs.

In certain embodiments, the particulate material is selected in a group comprising:
an organic material, including demineralized bone matrix, gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins, cellulose derivatives, or combinations thereof;
a ceramic material, including particles of calcium phosphate (CaP), calcium carbonate (CaCO₃), calcium sulfate (CaSO₄), or calcium hydroxide (Ca(OH)₂), or combinations thereof;
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example poly(propylene fumarate) (PPF) or poly(propylene fumarate-co-ethylene glycol) (P(PF-co-EG)), oligo(poly(ethylene glycol) fumarate) (OPF), poly (n-isopropylacrylamide) (PNIPPAAm), poly(aldehyde guluronate) (PAG), poly(n-vinyl pyrrolidone) (PNVP), or combinations thereof;
a gel, including a self-assembling oligopeptide gel, a hydrogel material, a microgel, a nanogel, a particulate gel, a hydrogel material, a thixotropic gel, a xerogel, a responsive gel, or combinations thereof;
a creamer;
and any combination thereof.

In some embodiments, the particulate material is gelatin or a ceramic material.

Another aspect of the invention relates to a composition comprising at least three miRNAs selected in selected in any one of Table 1, Table 2, Table 3, Table 4, Table 5, Table 6, Table 7, Table 8, Table 9, Table 10, Table 11 or Table 12, obtainable by a method according to the instant invention.

Definitions

Unless otherwise defined, all technical and scientific terms used herein have the same meaning as commonly understood by one of ordinary skill in the art to which this invention pertains. In case of conflict, the present definitions provided by the instant are to be considered as authentic.

In the present invention, the following terms have the following meanings:
The term “about” preceding a value means plus or less 10% of the value of said value. It is to be understood that the value to which the term “about” refers to is itself also specifically, and preferably, disclosed.
The term “comprises” is intended to mean “contains”, “encompasses” and “includes”. In some embodiments, the term “comprises” also encompasses the term “consists of”.
The term “tissue disorder” is intended to refer to any disturbance, unbalance, of the physiological function of a tissue. Non-limitative examples of symptoms observed in tissue disorders include injury, strain, infection, sprain, trauma, fissure, swelling, redness, edema, aching, tenderness, soreness, wound, necrosis, or any combination thereof. As used herein, terms such as “tissue disorder”, “tissue disease”, “tissue medical condition” are intended to be equivalent.
The term “regeneration” or “tissue regeneration” includes, but is not limited to the growth, generation, or reconstruction of new cells types or tissues following a treatment with a pharmaceutical composition according to the invention. In one embodiment, these cells types or tissues include but are not limited to osteogenic cells (e.g., osteoblasts, osteocytes), chondrocytes, epithelial cells, endothelial cells, fibroblasts, keratinocytes, cardiomyocytes, hematopoietic cells, hepatic cells, adipocytes, neural cells, and myotubes. As used herein, the term “regeneration” is envisioned as preventive and/or therapeutic treatment with the cell types aforementioned following injury, wounding, surgeries, congenital, degenerative, traumatic or non-traumatic symptoms or conditions, or other procedures that result in fissures, openings, depressions, wounds, and the like.
The term “repair” or “tissue repair” includes but is not limited to the healing process to reconstruct a healthy tissue from a diseased or dysfunctional tissue. In one embodiment, tissue repair includes skin repair, such as, e.g., would healing, scar formation and attenuation. In one embodiment tissue repair includes bone repair, such as, e.g., fracture reduction. In one embodiment, tissue repair includes cartilage repair. In one embodiment, tissue repair includes filling, bulking, supporting, enlarging, extending, or increasing the size or mass of body tissue.
The term “miRNA” or “miR” refers to a non-coding RNA of about 18 to about 25 nucleotides in length. These miRNAs could originate from multiple origins including: an individual gene encoding for a miRNA, from introns of protein coding gene, or from poly-cistronic transcript that often encode multiple, closely related miRNAs. In the following disclosure, the standard nomenclature system is applied, in which uncapitalized “mir-X” refers to the pre-miRNA (precursor), and capitalized “miR-X” refers to the mature form. When two mature miRNAs originate from opposite arms of the same pre-miRNA, they are denoted with a −3p or −5p suffix. In the following disclosure, unless otherwise specified, the use of the expression miR-X refers to the mature miRNA including both forms −3p and −5p, if any. Within the scope of the invention, the expressions microRNA, miRNA and miR designate the same compound.
The term “exosome” is intended to refer to the extracellular vesicles that are released from cells upon fusion of an intermediate endocytic compartment, the multivesicular body (MVB), with the plasma membrane. In other words, exosomes correspond to the intraluminal vesicles that are released into the extracellular milieu.
The term “secretion” refers to a physiologically active substance transported out of the cell in which it is synthesized. In one embodiment, the physiologically active substance may be any molecule, in particular a protein (such as a growth factor or a transcription factor) or a nucleic acid (such as a miRNA). As used herein, the term “secretion” includes both active and passive secretion. In this application, the term “active secretion” refers to the secretion of physiologically active substances by living cells, in particular mesenchymal stem cells and preferably adipose tissue-derived stem cells, out of the cells as a response to a stimulus, thereby diffusing into the environment of the cells, such as, e.g., the extracellular matrix. By “living cells”, it is meant herein cells presenting at least one of the following characteristics: growth and development, reproduction, homeostasis, response to stimuli, consumption, metabolism, excretion. In this application, the term “passive secretion” refers to physiologically active substances released by non-living cells or fragments or extracts thereof, out of the cells or fragments or extracts thereof in the absence of a stimulus, thereby diffusing into the environment of the originating cells or fragments or extracts thereof, such as, e.g., the extracellular matrix. By “non-living cells”, it is meant herein cells presenting none of the following characteristics: growth and development, reproduction, homeostasis, response to stimuli, consumption, metabolism, excretion (non-living cells or fragments or extracts thereof are, for example, dead cells or cellular extracts). Physiologically active substances that are actively or passively secreted may then diffuse into the tissue or organ in which the biomaterial comprising such extracellular matrix is administered.
The term “treatment”, “treating” or “alleviation” refers to therapeutic treatments wherein the object is to prevent or slow down (lessen) a tissue disorder, including a skin disorder, a bone disorder and/or a cartilage disorder. Those in need of treatment include those already with the disorder as well as those prone to have the disorder or those in whom the tissue disorder, including the skin, bone or cartilage defect is to be prevented. A subject is successfully “treated” for a tissue disorder, including a skin disorder, a bone disorder and/or a cartilage disorder if, after receiving a therapeutic amount of a composition according to the methods of the present invention, the individual shows observable and/or measurable reduction in, or absence of, one or more of the following: reduction in the tissue disorder, such as, e.g., a skin disorder, a bone disorder and/or a cartilage disorder and/or relief to some extent, one or more of the symptoms associated with the tissue disorder, including a skin disorder, a bone disorder and/or a cartilage disorder; reduced morbidity and mortality, and improvement in quality of life issues. The above parameters for assessing successful treatment and improvement in the disorder are readily measurable by routine procedures familiar to a physician.
The term “prevention” refers to preventing or avoiding the occurrence of symptom of a tissue disorder, such as, e.g., a skin disorder, a bone disorder and/or a cartilage disorder. In the present invention, the term “prevention” may refer to a secondary prevention, i.e., to the prevention of the re-occurrence of a symptom or a relapse of a tissue disorder, such as, e.g., a skin disorder, a bone disorder and/or a cartilage disorder. It may also refer, when the disease is cancer, such as a bone cancer, to the occurrence of metastases after the treatment and/or the removal of a tumor.
The term “effective amount” refers to an amount sufficient to promote beneficial or desired results including clinical results. An effective amount can be administered in one or more administration(s).
The term “pharmaceutically acceptable vehicle” refers to a vehicle that does not produce any adverse, allergic or other unwanted reactions when administered to an animal individual, preferably a human individual. It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory Offices, such as, e.g., the Food and Drug Administration (FDA) in the United States or the European Medicines Agency (EMA) in the European Union.
The term “individual” refers to a vertebrate animal, preferably a mammal, more preferably a human. Examples of individuals include humans, non-human primates, dogs, cats, mice, rats, horses, cows, sheep and transgenic species thereof. In one embodiment, an individual may be a “patient”, i.e., a warm-blooded animal, more preferably a human, who/which is awaiting the receipt of, or is receiving medical care or was/is/will be the object of a medical procedure, or is monitored for the development of a disease. In one embodiment, the individual is an adult (for example a human subject above the age of 18). In another embodiment, the individual is a child (for example a human subject below the age of 18). In one embodiment, the individual is a male. In another embodiment, the individual is a female.

Other definitions appear in context throughout this disclosure.

### DETAILED DESCRIPTION

It was shown in the publication WO2019/057862 that the disclosed biomaterial may be used for treating bone or cartilage disorders. The publication WO2020/058511 also described a biomaterial that may be used for treating tissue disorders. It has emerged from further characterization of said biomaterials that the cellular content or the secreted content is of primary importance for promoting tissue repair, including bone repair and cartilage repair. Noticeably, it was shown that growth factors, transcription factors, and factors that are involved in tissue formation, including bone formation or cartilage formation, together with various micro RNAs (miRNAs), may represent the active agents for tissue repairing and/or tissue regeneration.

MicroRNAs (miRNAs) are short (approximately 18- to 25-nucleotide long) non-coding RNAs that silence gene expression post-transcriptionally, principally by binding to 3′ untranslated regions (3′UTR) of target mRNAs. Mature miRNAs are known to be required for the normal differentiation and function of several cell types.

The state of the art has disclosed that miRNAs may be useful for therapy. For example, WO2014072468 disclosed activated blood serum preparation mixed with platelet-rich plasma comprising miRNAs, which has been considered as being of therapeutic use for cartilage regeneration. WO2015052526 disclosed stem cells microparticles and miRNAs isolated therefrom, and their use in therapy of diseases including fibrosis, cancer, rheumatoid arthritis, atherosclerosis. WO2017163132 disclosed miRNAs-comprising exosomes that are secreted by umbilical cord blood mononuclear cells, which may be useful to treat wounds, in particular chronic wounds.

Without wishing to be bound to a theory, the inventors identified cocktails of miRNAs that may promote tissue regeneration, including osteogenesis and/or chondrogenesis, and be of therapeutic value in the prevention and/or the treatment of tissue disorders, including skin disorders, bone disorders and/or cartilage disorders. In practice, we discovered this miRNA cocktail may be extracted and purified from biomaterials produced by contacting (i) suitable differentiated cells having tissue regenerating and/or repairing properties, e.g., capable to undergo osteo- and/or chondro-induction with (ii) a particulate material, preferably gelatin or a ceramic material, in a culture medium allowing cell proliferation and secretion of an extracellular matrix. Said biomaterials may be characterized by their original miRNAs' content, originating from the cells themselves and/or from exosomes, or exosome-like vesicles, that are secreted by said cells.

The recitation of an embodiment below includes that embodiment as any single embodiment or in combination with any other embodiments or portions thereof and the recited embodiments are applicable to one or more of the aspects recited below. Other features and advantages of the invention will be apparent from the detailed description and from the claims. Thus, other aspects and embodiments of the invention are described in the following disclosure and are within the ambit of the invention.

The invention pertains to a pharmaceutical composition comprising (i) a therapeutically effective amount of at least one miRNA selected in any one of Table 1, Table 2, Table 3, Table 4, Table 5, Table 6, Table 7, Table 8, Table 9, Table 10, Table 11 or Table 12, and (ii) a pharmaceutical acceptable vehicle.

The invention also pertains to a pharmaceutical composition comprising (i) a therapeutically effective amount of at least three miRNAs selected in any one of Table 1, Table 2, Table 3, Table 4, Table 5, Table 6, Table 7, Table 8, Table 9, Table 10, Table 11 or Table 12, and (ii) a pharmaceutical acceptable vehicle.

As used herein, the expression “therapeutically effective amount” is intended to refer to a quantity of the active ingredient(s) that is sufficient to promote a physiological benefit to an individual in need thereof.

As used herein, the term “at least three miRNAs” includes 3, 4, 5, 6, 7, 8, 9, 10, 11, 12, 13, 14, 15, 16, 17, 18, 19, 20 or more miRNAs. In some embodiments, the combination of at least three miRNAs according to the invention are referred to as a cocktail of miRNAs.

Criteria and conventions for miRNA identification and nomenclature have been described in Ambros et al. (A uniform system for microRNA annotation. RNA 2003 9(3):277-279). The miRNAs sequences may be easily retrieved from the miRbase database (http://www.mirbase.org/) or the miRDB database (http://www.mirdb.org/).

TABLE 1
miRNAs according to the invention

| | | | |
|---|---|---|---|
| hsa-let-7a-5p | hsa-miR-199a-3p | hsa-miR-10a-5p | hsa-miR-411-5p |
| hsa-let-7b-5p | hsa-miR-145-5p | hsa-miR-495-3p | hsa-miR-505-5p |
| hsa-let-7f-5p | hsa-miR-30a-3p | hsa-miR-425-5p | hsa-miR-664a-3p |
| hsa-miR-24-3p | hsa-miR-382-5p | hsa-miR-2053 | hsa-miR-26a-5p |
| hsa-miR-21-5p | hsa-miR-19b-3p | hsa-miR-5096 | hsa-miR-377-3p |
| hsa-miR-23b-3p | hsa-miR-210-3p | hsa-miR-494-3p | hsa-miR-485-3p |
| hsa-miR-1273g-3p | hsa-miR-619-5p | hsa-miR-27a-3p | hsa-miR-590-3p |
| hsa-miR-574-3p | hsa-miR-17-5p | hsa-miR-4449 | hsa-miR-99a-3p |
| hsa-miR-25-3p | hsa-miR-193a-5p | hsa-miR-532-3p | hsa-miR-143-3p |
| hsa-let-7e-5p | hsa-miR-320b | hsa-miR-532-5p | hsa-miR-26b-3p |
| hsa-miR-214-3p | hsa-miR-193b-5p | hsa-miR-126-5p | hsa-miR-3607-5p |
| hsa-miR-199a-5p | hsa-miR-320a | hsa-miR-30c-5p | hsa-miR-3651 |
| hsa-miR-196a-5p | hsa-miR-151a-3p | hsa-miR-130b-3p | hsa-miR-374a-3p |
| hsa-miR-199b-5p | hsa-let-7a-3p | hsa-miR-136-3p | hsa-miR-376a-3p |
| hsa-miR-221-3p | hsa-miR-30e-3p | hsa-miR-15b-3p | hsa-miR-485-5p |
| hsa-miR-424-5p | hsa-miR-22-3p | hsa-miR-29b-1-5p | hsa-miR-103b |
| hsa-miR-23a-3p | hsa-miR-99b-5p | hsa-miR-99b-3p | hsa-miR-126-3p |
| hsa-let-7c-5p | hsa-miR-625-3p | hsa-miR-127-3p | hsa-miR-149-5p |
| hsa-miR-199b-3p | hsa-miR-4668-5p | hsa-miR-134-5p | hsa-miR-193b-3p |
| hsa-miR-191-5p | hsa-miR-29b-3p | hsa-miR-324-5p | hsa-miR-223-3p |
| hsa-miR-574-5p | hsa-miR-423-3p | hsa-miR-3605-3p | hsa-miR-340-3p |
| hsa-miR-424-3p | hsa-miR-376c-3p | hsa-miR-101-3p | hsa-miR-369-5p |
| hsa-miR-423-5p | hsa-let-7b-3p | hsa-miR-103a-3p | hsa-miR-6724-5p |
| hsa-miR-342-3p | hsa-miR-3074-5p | hsa-miR-1246 | hsa-miR-7847-3p |
| hsa-let-7d-3p | hsa-miR-98-5p | hsa-miR-138-5p | hsa-miR-874-3p |
| hsa-miR-130a-3p | hsa-miR-185-5p | hsa-miR-190a-5p | hsa-miR-3653-5p |
| hsa-miR-3184-3p | hsa-miR-19a-3p | hsa-miR-24-2-5p | hsa-miR-664b-3p |
| hsa-miR-222-3p | hsa-miR-34a-5p | hsa-miR-26a-2-3p | hsa-miR-664b-5p |
| hsa-let-7g-5p | hsa-miR-374c-3p | hsa-miR-301a-3p | hsa-miR-6516-3p |
| hsa-miR-125a-5p | hsa-miR-181a-5p | hsa-miR-98-3p | hsa-let-7i-3p |
| hsa-let-7d-5p | hsa-miR-328-3p | hsa-miR-1273a | hsa-miR-154-5p |
| hsa-miR-29a-3p | hsa-miR-92b-3p | hsa-miR-28-5p | hsa-miR-664a-5p |
| hsa-let-7i-5p | hsa-miR-335-5p | hsa-miR-34a-3p | hsa-miR-1291 |
| hsa-miR-146b-5p | hsa-let-7f-1-3p | hsa-miR-425-3p | hsa-miR-140-5p |
| hsa-miR-4454 | hsa-miR-196b-5p | hsa-miR-505-3p | hsa-miR-3609 |
| hsa-miR-28-3p | hsa-miR-3613-3p | hsa-miR-34b-3p | hsa-miR-4461 |
| hsa-miR-92a-3p | hsa-miR-23a-5p | hsa-miR-361-3p | hsa-miR-3613-5p |
| hsa-miR-125b-5p | hsa-miR-374b-5p | hsa-miR-10b-5p | hsa-miR-663b |
| hsa-miR-337-3p | hsa-miR-660-5p | hsa-miR-1306-5p | hsa-miR-378a-3p |
| hsa-miR-93-5p | hsa-miR-186-5p | hsa-miR-22-5p | hsa-miR-454-3p |
| hsa-miR-409-3p | | | |

In some embodiments, the at least three miRNAs are selected in a group comprising hsa-let-7a-5p, hsa-miR-199a-3p, hsa-miR-10a-5p, hsa-miR-411-5p, hsa-let-7b-5p, hsa-miR-145-5p, hsa-miR-495-3p, hsa-miR-505-5p, hsa-let-7f-5p, hsa-miR-30a-3p, hsa-miR-425-5p, hsa-miR-664a-3p, hsa-miR-24-3p, hsa-miR-382-5p, hsa-miR-2053, hsa-miR-26a-5p, hsa-miR-21-5p, hsa-miR-19b-3p, hsa-miR-5096, hsa-miR-377-3p, hsa-miR-23b-3p, hsa-miR-210-3p, hsa-miR-494-3p, hsa-miR-485-3p, hsa-miR-1273g-3p, hsa-miR-619-5p, hsa-miR-27a-3p, hsa-miR-590-3p, hsa-miR-574-3p, hsa-miR-17-5p, hsa-miR-4449, hsa-miR-99a-3p, hsa-miR-25-3p, hsa-miR-193a-5p, hsa-miR-532-3p, hsa-miR-143-3p, hsa-let-7e-5p, hsa-miR-320b, hsa-miR-532-5p, hsa-miR-26b-3p, hsa-miR-214-3p, hsa-miR-193b-5p, hsa-miR-126-5p, hsa-miR-3607-5p, hsa-miR-199a-5p, hsa-miR-320a, hsa-miR-30c-5p, hsa-miR-3651, hsa-miR-196a-5p, hsa-miR-151a-3p, hsa-miR-130b-3p, hsa-miR-374a-3p, hsa-miR-199b-5p, hsa-let-7a-3p, hsa-miR-136-3p, hsa-miR-376a-3p, hsa-miR-221-3p, hsa-miR-30e-3p, hsa-miR-15b-3p, hsa-miR-485-5p, hsa-miR-424-5p, hsa-miR-22-3p, hsa-miR-29b-1-5p, hsa-miR-103b, hsa-miR-23a-3p, hsa-miR-99b-5p, hsa-miR-99b-3p, hsa-miR-126-3p, hsa-let-7c-5p, hsa-miR-625-3p, hsa-miR-127-3p, hsa-miR-149-5p, hsa-miR-199b-3p, hsa-miR-4668-5p, hsa-miR-134-5p, hsa-miR-193b-3p, hsa-miR-191-5p, hsa-miR-29b-3p, hsa-miR-324-5p, hsa-miR-223-3p, hsa-miR-574-5p, hsa-miR-423-3p, hsa-miR-3605-3p, hsa-miR-340-3p, hsa-miR-424-3p, hsa-miR-376c-3p, hsa-miR-101-3p, hsa-miR-369-5p, hsa-miR-423-5p, hsa-let-7b-3p, hsa-miR-103a-3p, hsa-miR-6724-5p, hsa-miR-342-3p, hsa-miR-3074-5p, hsa-miR-1246, hsa-miR-7847-3p, hsa-let-7d-3p, hsa-miR-98-5p, hsa-miR-138-5p, hsa-miR-874-3p, hsa-miR-130a-3p, hsa-miR-185-5p, hsa-miR-190a-5p, hsa-miR-3653-5p, hsa-miR-3184-3p, hsa-miR-19a-3p, hsa-miR-24-2-5p, hsa-miR-664b-3p, hsa-miR-222-3p, hsa-miR-34a-5p, hsa-miR-26a-2-3p, hsa-miR-664b-5p, hsa-let-7g-5p, hsa-miR-374c-3p, hsa-miR-301a-3p, hsa-miR-6516-3p, hsa-miR-125a-5p, hsa-miR-181a-5p, hsa-miR-98-3p, hsa-let-7i-3p, hsa-let-7d-5p, hsa-miR-328-3p, hsa-miR-1273a, hsa-miR-154-5p, hsa-miR-29a-3p, hsa-miR-92b-3p, hsa-miR-28-5p, hsa-miR-664a-5p, hsa-let-7i-5p, hsa-miR-335-5p, hsa-miR-34a-3p, hsa-miR-1291, hsa-miR-146b-5p, hsa-let-7f-1-3p, hsa-miR-425-3p, hsa-miR-140-5p, hsa-miR-4454, hsa-miR-196b-5p, hsa-miR-505-3p, hsa-miR-3609, hsa-miR-28-3p, hsa-miR-3613-3p, hsa-miR-34b-3p, hsa-miR-4461, hsa-miR-92a-3p, hsa-miR-23a-5p, hsa-miR-361-3p, hsa-miR-3613-5p, hsa-miR-125b-5p, hsa-miR-374b-5p, hsa-miR-10b-5p, hsa-miR-663b, hsa-miR-337-3p, hsa-miR-660-5p, hsa-miR-1306-5p, hsa-miR-378a-3p, hsa-miR-93-5p, hsa-miR-186-5p, hsa-miR-22-5p, hsa-miR-454-3p, hsa-miR-409-3p, and a combination thereof.

TABLE 2
miRNAs according to the invention

| | | | |
|---|---|---|---|
| hsa-let-7a-5p | hsa-miR-30a-3p | hsa-miR-103a-3p | hsa-miR-542-3p |
| hsa-let-7b-5p | hsa-miR-320b | hsa-miR-19a-3p | hsa-miR-663a |
| hsa-miR-24-3p | hsa-miR-193a-5p | hsa-miR-126-5p | hsa-miR-101-3p |
| hsa-miR-21-5p | hsa-miR-382-5p | hsa-miR-2053 | hsa-miR-143-3p |
| hsa-let-7f-5p | hsa-miR-423-3p | hsa-miR-29b-1-5p | hsa-miR-21-3p |
| hsa-miR-574-3p | hsa-miR-17-5p | hsa-miR-3648 | hsa-miR-224-5p |
| hsa-miR-23b-3p | hsa-miR-19b-3p | hsa-miR-374a-3p | hsa-miR-26a-5p |
| hsa-miR-1273g-3p | hsa-miR-92b-3p | hsa-miR-454-3p | hsa-miR-27a-5p |
| hsa-miR-25-3p | hsa-miR-320a | hsa-miR-532-3p | hsa-miR-324-5p |
| hsa-miR-199a-5p | hsa-miR-3074-5p | hsa-miR-136-3p | hsa-miR-340-3p |
| hsa-miR-196a-5p | hsa-miR-376c-3p | hsa-miR-361-3p | hsa-miR-379-5p |
| hsa-miR-214-3p | hsa-let-7b-3p | hsa-miR-1246 | hsa-miR-409-5p |
| hsa-miR-125a-5p | hsa-miR-625-3p | hsa-miR-130b-3p | hsa-miR-543 |
| hsa-miR-221-3p | hsa-miR-99b-5p | hsa-miR-134-5p | hsa-miR-5787 |
| hsa-miR-222-3p | hsa-miR-34a-5p | hsa-miR-154-5p | hsa-miR-6089 |
| hsa-let-7e-5p | hsa-miR-5096 | hsa-miR-34a-3p | hsa-miR-127-3p |
| hsa-miR-191-5p | hsa-miR-30e-3p | hsa-miR-576-5p | hsa-miR-149-5p |
| hsa-miR-199b-3p | hsa-miR-22-3p | hsa-miR-874-3p | hsa-miR-181c-5p |
| hsa-miR-342-3p | hsa-miR-151a-3p | hsa-miR-100-5p | hsa-miR-193b-3p |
| hsa-miR-23a-3p | hsa-miR-186-5p | hsa-miR-103b | hsa-miR-222-5p |
| hsa-miR-424-3p | hsa-miR-193b-5p | hsa-miR-1273a | hsa-miR-3613-5p |
| hsa-miR-28-3p | hsa-miR-328-3p | hsa-miR-1306-5p | hsa-miR-365b-3p |
| hsa-let-7g-5p | hsa-miR-4449 | hsa-miR-138-5p | hsa-miR-3960 |
| hsa-miR-92a-3p | hsa-miR-27a-3p | hsa-miR-15b-3p | hsa-miR-485-3p |
| hsa-miR-424-5p | hsa-miR-30c-5p | hsa-miR-26b-3p | hsa-miR-6087 |
| hsa-let-7d-3p | hsa-miR-494-3p | hsa-miR-10b-5p | hsa-miR-92a-1-5p |
| hsa-miR-4454 | hsa-miR-98-5p | hsa-miR-22-5p | hsa-miR-3607-5p |
| hsa-miR-146b-5p | hsa-miR-10a-5p | hsa-miR-3613-3p | hsa-miR-3653-5p |
| hsa-miR-423-5p | hsa-miR-29b-3p | hsa-miR-655-3p | hsa-miR-664b-5p |
| hsa-miR-29a-3p | hsa-miR-374b-5p | hsa-miR-7-1-3p | hsa-miR-664b-3p |
| hsa-miR-574-5p | hsa-miR-335-5p | hsa-miR-23a-5p | hsa-miR-6516-3p |
| hsa-miR-199b-5p | hsa-miR-374c-3p | hsa-miR-24-2-5p | hsa-miR-1291 |
| hsa-miR-125b-5p | hsa-miR-425-5p | hsa-miR-3605-3p | hsa-let-7i-3p |
| hsa-miR-3184-3p | hsa-miR-181a-5p | hsa-miR-6832-3p | hsa-miR-455-3p |
| hsa-let-7c-5p | hsa-miR-196b-5p | hsa-miR-146a-5p | hsa-miR-671-5p |
| hsa-miR-337-3p | hsa-let-7f-1-3p | hsa-miR-16-2-3p | hsa-miR-1271-5p |
| hsa-let-7d-5p | hsa-miR-4668-5p | hsa-miR-181b-5p | hsa-miR-4461 |
| hsa-miR-145-5p | hsa-miR-660-5p | hsa-miR-26a-2-3p | hsa-miR-6724-5p |
| hsa-miR-93-5p | hsa-miR-664a-3p | hsa-miR-376a-3p | hsa-miR-190a-5p |
| hsa-miR-619-5p | hsa-miR-185-5p | hsa-miR-539-5p | hsa-miR-3609 |
| hsa-miR-130a-3p | hsa-miR-3651 | hsa-miR-708-5p | hsa-miR-411-5p |
| hsa-let-7i-5p | hsa-miR-495-3p | hsa-miR-98-3p | hsa-miR-425-3p |
| hsa-miR-409-3p | hsa-let-7a-3p | hsa-miR-1237-5p | hsa-miR-4485-3p |
| hsa-miR-210-3p | hsa-miR-28-5p | hsa-miR-223-3p | hsa-miR-532-5p |
| hsa-miR-199a-3p | hsa-miR-99b-3p | | |

In some embodiments, at least three miRNAs are selected in a group comprising or consisting of hsa-let-7a-5p, hsa-miR-30a-3p, hsa-miR-103a-3p, hsa-miR-542-3p, hsa-let-7b-5p, hsa-miR-320b, hsa-miR-19a-3p, hsa-miR-663a, hsa-miR-24-3p, hsa-miR-193a-5p, hsa-miR-126-5p, hsa-miR-101-3p, hsa-miR-21-5p, hsa-miR-382-5p, hsa-miR-2053, hsa-miR-143-3p, hsa-let-7f-5p, hsa-miR-423-3p, hsa-miR-29b-1-5p, hsa-miR-21-3p, hsa-miR-574-3p, hsa-miR-17-5p, hsa-miR-3648, hsa-miR-224-5p, hsa-miR-23b-3p, hsa-miR-19b-3p, hsa-miR-374a-3p, hsa-miR-26a-5p, hsa-miR-1273g-3p, hsa-miR-92b-3p, hsa-miR-454-3p, hsa-miR-27a-5p, hsa-miR-25-3p, hsa-miR-320a, hsa-miR-532-3p, hsa-miR-324-5p, hsa-miR-199a-5p, hsa-miR-3074-5p, hsa-miR-136-3p, hsa-miR-340-3p, hsa-miR-196a-5p, hsa-miR-376c-3p, hsa-miR-361-3p, hsa-miR-379-5p, hsa-miR-214-3p, hsa-let-7b-3p, hsa-miR-1246, hsa-miR-409-5p, hsa-miR-125a-5p, hsa-miR-625-3p, hsa-miR-130b-3p, hsa-miR-543, hsa-miR-221-3p, hsa-miR-99b-5p, hsa-miR-134-5p, hsa-miR-5787, hsa-miR-222-3p, hsa-miR-34a-5p, hsa-miR-154-5p, hsa-miR-6089, hsa-let-7e-5p, hsa-miR-5096, hsa-miR-34a-3p, hsa-miR-127-3p, hsa-miR-191-5p, hsa-miR-30e-3p, hsa-miR-576-5p, hsa-miR-149-5p, hsa-miR-199b-3p, hsa-miR-22-3p, hsa-miR-874-3p, hsa-miR-181c-5p, hsa-miR-342-3p, hsa-miR-151a-3p, hsa-miR-100-5p, hsa-miR-193b-3p, hsa-miR-23a-3p, hsa-miR-186-5p, hsa-miR-103b, hsa-miR-222-5p, hsa-miR-424-3p, hsa-miR-193b-5p, hsa-miR-1273a, hsa-miR-3613-5p, hsa-miR-28-3p, hsa-miR-328-3p, hsa-miR-1306-5p, hsa-miR-365b-3p, hsa-let-7g-5p, hsa-miR-4449, hsa-miR-138-5p, hsa-miR-3960, hsa-miR-92a-3p, hsa-miR-27a-3p, hsa-miR-15b-3p, hsa-miR-485-3p, hsa-miR-424-5p, hsa-miR-30c-5p, hsa-miR-26b-3p, hsa-miR-6087, hsa-let-7d-3p, hsa-miR-494-3p, hsa-miR-10b-5p, hsa-miR-92a-1-5p, hsa-miR-4454, hsa-miR-98-5p, hsa-miR-22-5p, hsa-miR-3607-5p, hsa-miR-146b-5p, hsa-miR-10a-5p, hsa-miR-3613-3p, hsa-miR-3653-5p, hsa-miR-423-5p, hsa-miR-29b-3p, hsa-miR-655-3p, hsa-miR-664b-5p, hsa-miR-29a-3p, hsa-miR-374b-5p, hsa-miR-7-1-3p, hsa-miR-664b-3p, hsa-miR-574-5p, hsa-miR-335-5p, hsa-miR-23a-5p, hsa-miR-6516-3p, hsa-miR-199b-5p, hsa-miR-374c-3p, hsa-miR-24-2-5p, hsa-miR-1291, hsa-miR-125b-5p, hsa-miR-425-5p, hsa-miR-3605-3p, hsa-let-7i-3p, hsa-miR-3184-3p, hsa-miR-181a-5p, hsa-miR-6832-3p, hsa-miR-455-3p, hsa-let-7c-5p, hsa-miR-196b-5p, hsa-miR-146a-5p, hsa-miR-671-5p, hsa-miR-337-3p, hsa-let-7f-1-3p, hsa-miR-16-2-3p, hsa-miR-1271-5p, hsa-let-7d-5p, hsa-miR-4668-5p, hsa-miR-18 lb-5p, hsa-miR-4461, hsa-miR-145-5p, hsa-miR-660-5p, hsa-miR-26a-2-3p, hsa-miR-6724-5p, hsa-miR-93-5p, hsa-miR-664a-3p, hsa-miR-376a-3p, hsa-miR-190a-5p, hsa-miR-619-5p, hsa-miR-185-5p, hsa-miR-539-5p, hsa-miR-3609, hsa-miR-130a-3p, hsa-miR-3651, hsa-miR-708-5p, hsa-miR-411-5p, hsa-let-7i-5p, hsa-miR-495-3p, hsa-miR-98-3p, hsa-miR-425-3p, hsa-miR-409-3p, hsa-let-7a-3p, hsa-miR-1237-5p, hsa-miR-4485-3p, hsa-miR-210-3p, hsa-miR-28-5p, hsa-miR-223-3p, hsa-miR-532-5p, hsa-miR-199a-3p, hsa-miR-99b-3p and a combination thereof.

TABLE 3
miRNAs according to the invention

| | | | |
|---|---|---|---|
| hsa-let-7a-5p | hsa-miR-92a-3p | hsa-miR-92b-3p | hsa-miR-24-2-5p |
| hsa-let-7b-5p | hsa-miR-125b-5p | hsa-miR-335-5p | hsa-miR-26a-2-3p |
| hsa-let-7f-5p | hsa-miR-337-3p | hsa-let-7f-1-3p | hsa-miR-301a-3p |
| hsa-miR-24-3p | hsa-miR-93-5p | hsa-miR-196b-5p | hsa-miR-98-3p |
| hsa-miR-21-5p | hsa-miR-409-3p | hsa-miR-3613-3p | hsa-miR-1273a |
| hsa-miR-23b-3p | hsa-miR-199a-3p | hsa-miR-23a-5p | hsa-miR-28-5p |
| hsa-miR-1273g-3p | hsa-miR-145-5p | hsa-miR-374b-5p | hsa-miR-34a-3p |
| hsa-miR-574-3p | hsa-miR-30a-3p | hsa-miR-660-5p | hsa-miR-425-3p |
| hsa-miR-25-3p | hsa-miR-382-5p | hsa-miR-186-5p | hsa-miR-505-3p |
| hsa-let-7e-5p | hsa-miR-19b-3p | hsa-miR-454-3p | hsa-miR-34b-3p |
| hsa-miR-214-3p | hsa-miR-210-3p | hsa-miR-10a-5p | hsa-miR-361-3p |
| hsa-miR-199a-5p | hsa-miR-619-5p | hsa-miR-495-3p | hsa-miR-10b-5p |
| hsa-miR-196a-5p | hsa-miR-17-5p | hsa-miR-425-5p | hsa-miR-1306-5p |
| hsa-miR-199b-5p | hsa-miR-193a-5p | hsa-miR-2053 | hsa-miR-22-5p |
| hsa-miR-221-3p | hsa-miR-320b | hsa-miR-5096 | hsa-miR-378a-3p |
| hsa-miR-424-5p | hsa-miR-193b-5p | hsa-miR-494-3p | hsa-miR-411-5p |
| hsa-miR-23a-3p | hsa-miR-320a | hsa-miR-27a-3p | hsa-miR-505-5p |
| hsa-let-7c-5p | hsa-miR-151a-3p | hsa-miR-4449 | hsa-miR-664a-3p |
| hsa-miR-199b-3p | hsa-let-7a-3p | hsa-miR-532-3p | hsa-miR-26a-5p |
| hsa-miR-191-5p | hsa-miR-30e-3p | hsa-miR-532-5p | hsa-miR-377-3p |
| hsa-miR-574-5p | hsa-miR-22-3p | hsa-miR-126-5p | hsa-miR-485-3p |
| hsa-miR-424-3p | hsa-miR-99b-5p | hsa-miR-30c-5p | hsa-miR-590-3p |
| hsa-miR-423-5p | hsa-miR-625-3p | hsa-miR-130b-3p | hsa-miR-99a-3p |
| hsa-miR-342-3p | hsa-miR-4668-5p | hsa-miR-136-3p | hsa-miR-143-3p |
| hsa-let-7d-3p | hsa-miR-29b-3p | hsa-miR-15b-3p | hsa-miR-26b-3p |
| hsa-miR-130a-3p | hsa-miR-423-3p | hsa-miR-29b-1-5p | hsa-miR-3607-5p |
| hsa-miR-3184-3p | hsa-miR-376c-3p | hsa-miR-99b-3p | hsa-miR-3651 |
| hsa-miR-222-3p | hsa-let-7b-3p | hsa-miR-127-3p | hsa-miR-374a-3p |
| hsa-let-7g-5p | hsa-miR-3074-5p | hsa-miR-134-5p | hsa-miR-376a-3p |
| hsa-miR-125a-5p | hsa-miR-98-5p | hsa-miR-324-5p | hsa-miR-485-5p |
| hsa-let-7d-5p | hsa-miR-185-5p | hsa-miR-3605-3p | hsa-miR-103b |
| hsa-miR-29a-3p | hsa-miR-19a-3p | hsa-miR-101-3p | hsa-miR-126-3p |
| hsa-let-7i-5p | hsa-miR-34a-5p | hsa-miR-103a-3p | hsa-miR-149-5p |
| hsa-miR-146b-5p | hsa-miR-374c-3p | hsa-miR-1246 | hsa-miR-193b-3p |
| hsa-miR-4454 | hsa-miR-181a-5p | hsa-miR-138-5p | hsa-miR-223-3p |
| hsa-miR-28-3p | hsa-miR-328-3p | hsa-miR-190a-5p | hsa-miR-340-3p |
| hsa-miR-874-3p | hsa-miR-7847-3p | hsa-miR-6724-5p | hsa-miR-369-5p |

In some embodiments, the at least three miRNAs are selected in a group comprising hsa-let-7a-5p, hsa-miR-92a-3p, hsa-miR-92b-3p, hsa-miR-24-2-5p, hsa-let-7b-5p, hsa-miR-125b-5p, hsa-miR-335-5p, hsa-miR-26a-2-3p, hsa-let-7f-5p, hsa-miR-337-3p, hsa-let-7f-1-3p, hsa-miR-301a-3p, hsa-miR-24-3p, hsa-miR-93-5p, hsa-miR-196b-5p, hsa-miR-98-3p, hsa-miR-21-5p, hsa-miR-409-3p, hsa-miR-3613-3p, hsa-miR-1273a, hsa-miR-23b-3p, hsa-miR-199a-3p, hsa-miR-23a-5p, hsa-miR-28-5p, hsa-miR-1273g-3p, hsa-miR-145-5p, hsa-miR-374b-5p, hsa-miR-34a-3p, hsa-miR-574-3p, hsa-miR-30a-3p, hsa-miR-660-5p, hsa-miR-425-3p, hsa-miR-25-3p, hsa-miR-382-5p, hsa-miR-186-5p, hsa-miR-505-3p, hsa-let-7e-5p, hsa-miR-19b-3p, hsa-miR-454-3p, hsa-miR-34b-3p, hsa-miR-214-3p, hsa-miR-210-3p, hsa-miR-10a-5p, hsa-miR-361-3p, hsa-miR-199a-5p, hsa-miR-619-5p, hsa-miR-495-3p, hsa-miR-10b-5p, hsa-miR-196a-5p, hsa-miR-17-5p, hsa-miR-425-5p, hsa-miR-1306-5p, hsa-miR-199b-5p, hsa-miR-193a-5p, hsa-miR-2053, hsa-miR-22-5p, hsa-miR-221-3p, hsa-miR-320b, hsa-miR-5096, hsa-miR-378a-3p, hsa-miR-424-5p, hsa-miR-193b-5p, hsa-miR-494-3p, hsa-miR-411-5p, hsa-miR-23a-3p, hsa-miR-320a, hsa-miR-27a-3p, hsa-miR-505-5p, hsa-let-7c-5p, hsa-miR-151a-3p, hsa-miR-4449, hsa-miR-664a-3p, hsa-miR-199b-3p, hsa-let-7a-3p, hsa-miR-532-3p, hsa-miR-26a-5p, hsa-miR-191-5p, hsa-miR-30e-3p, hsa-miR-532-5p, hsa-miR-377-3p, hsa-miR-574-5p, hsa-miR-22-3p, hsa-miR-126-5p, hsa-miR-485-3p, hsa-miR-424-3p, hsa-miR-99b-5p, hsa-miR-30c-5p, hsa-miR-590-3p, hsa-miR-423-5p, hsa-miR-625-3p, hsa-miR-130b-3p, hsa-miR-99a-3p, hsa-miR-342-3p, hsa-miR-4668-5p, hsa-miR-136-3p, hsa-miR-143-3p, hsa-let-7d-3p, hsa-miR-29b-3p, hsa-miR-15b-3p, hsa-miR-26b-3p, hsa-miR-130a-3p, hsa-miR-423-3p, hsa-miR-29b-1-5p, hsa-miR-3607-5p, hsa-miR-3184-3p, hsa-miR-376c-3p, hsa-miR-99b-3p, hsa-miR-3651, hsa-miR-222-3p, hsa-let-7b-3p, hsa-miR-127-3p, hsa-miR-374a-3p, hsa-let-7g-5p, hsa-miR-3074-5p, hsa-miR-134-5p, hsa-miR-376a-3p, hsa-miR-125a-5p, hsa-miR-98-5p, hsa-miR-324-5p, hsa-miR-485-5p, hsa-let-7d-5p, hsa-miR-185-5p, hsa-miR-3605-3p, hsa-miR-103b, hsa-miR-29a-3p, hsa-miR-19a-3p, hsa-miR-101-3p, hsa-miR-126-3p, hsa-let-7i-5p, hsa-miR-34a-5p, hsa-miR-103a-3p, hsa-miR-149-5p, hsa-miR-146b-5p, hsa-miR-374c-3p, hsa-miR-1246, hsa-miR-193b-3p, hsa-miR-4454, hsa-miR-181a-5p, hsa-miR-138-5p, hsa-miR-223-3p, hsa-miR-28-3p, hsa-miR-328-3p, hsa-miR-190a-5p, hsa-miR-340-3p, hsa-miR-874-3p, hsa-miR-7847-3p, hsa-miR-6724-5p, hsa-miR-369-5p, and a combination thereof.

TABLE 4
miRNAs according to the invention

| | | | |
|---|---|---|---|
| hsa-let-7a-5p | hsa-let-7i-5p | hsa-miR-660-5p | hsa-miR-6832-3p |
| hsa-let-7b-5p | hsa-miR-409-3p | hsa-miR-664a-3p | hsa-miR-146a-5p |
| hsa-miR-24-3p | hsa-miR-210-3p | hsa-miR-185-5p | hsa-miR-16-2-3p |
| hsa-miR-21-5p | hsa-miR-199a-3p | hsa-miR-3651 | hsa-miR-181b-5p |
| hsa-let-7f-5p | hsa-miR-30a-3p | hsa-miR-495-3p | hsa-miR-26a-2-3p |
| hsa-miR-574-3p | hsa-miR-320b | hsa-let-7a-3p | hsa-miR-376a-3p |
| hsa-miR-23b-3p | hsa-miR-193a-5p | hsa-miR-28-5p | hsa-miR-539-5p |
| hsa-miR-1273g-3p | hsa-miR-382-5p | hsa-miR-99b-3p | hsa-miR-708-5p |
| hsa-miR-25-3p | hsa-miR-423-3p | hsa-miR-103a-3p | hsa-miR-98-3p |
| hsa-miR-199a-5p | hsa-miR-17-5p | hsa-miR-19a-3p | hsa-miR-1237-5p |
| hsa-miR-196a-5p | hsa-miR-19b-3p | hsa-miR-126-5p | hsa-miR-223-3p |
| hsa-miR-214-3p | hsa-miR-92b-3p | hsa-miR-2053 | hsa-miR-532-5p |
| hsa-miR-125a-5p | hsa-miR-320a | hsa-miR-29b-1-5p | hsa-miR-542-3p |
| hsa-miR-221-3p | hsa-miR-3074-5p | hsa-miR-3648 | hsa-miR-663a |
| hsa-miR-222-3p | hsa-miR-376c-3p | hsa-miR-374a-3p | hsa-miR-101-3p |
| hsa-let-7e-5p | hsa-let-7b-3p | hsa-miR-454-3p | hsa-miR-143-3p |
| hsa-miR-191 -5p | hsa-miR-625-3p | hsa-miR-532-3p | hsa-miR-21-3p |
| hsa-miR-199b-3p | hsa-miR-99b-5p | hsa-miR-136-3p | hsa-miR-224-5p |
| hsa-miR-342-3p | hsa-miR-34a-5p | hsa-miR-361-3p | hsa-miR-26a-5p |
| hsa-miR-23a-3p | hsa-miR-5096 | hsa-miR-1246 | hsa-miR-27a-5p |
| hsa-miR-424-3p | hsa-miR-30e-3p | hsa-miR-130b-3p | hsa-miR-324-5p |
| hsa-miR-28-3p | hsa-miR-22-3p | hsa-miR-134-5p | hsa-miR-340-3p |
| hsa-let-7g-5p | hsa-miR-151a-3p | hsa-miR-154-5p | hsa-miR-379-5p |
| hsa-miR-92a-3p | hsa-miR-186-5p | hsa-miR-34a-3p | hsa-miR-409-5p |
| hsa-miR-424-5p | hsa-miR-193b-5p | hsa-miR-576-5p | hsa-miR-543 |
| hsa-let-7d-3p | hsa-miR-328-3p | hsa-miR-874-3p | hsa-miR-5787 |
| hsa-miR-4454 | hsa-miR-4449 | hsa-miR-100-5p | hsa-miR-6089 |
| hsa-miR-146b-5p | hsa-miR-27a-3p | hsa-miR-103b | hsa-miR-127-3p |
| hsa-miR-423-5p | hsa-miR-30c-5p | hsa-miR-1273a | hsa-miR-149-5p |
| hsa-miR-29a-3p | hsa-miR-494-3p | hsa-miR-1306-5p | hsa-miR-181c-5p |
| hsa-miR-574-5p | hsa-miR-98-5p | hsa-miR-138-5p | hsa-miR-193b-3p |
| hsa-miR-199b-5p | hsa-miR-10a-5p | hsa-miR-15b-3p | hsa-miR-222-5p |
| hsa-miR-125b-5p | hsa-miR-29b-3p | hsa-miR-26b-3p | hsa-miR-3613-5p |
| hsa-miR-3184-3p | hsa-miR-374b-5p | hsa-miR-10b-5p | hsa-miR-365b-3p |
| hsa-let-7c-5p | hsa-miR-335-5p | hsa-miR-22-5p | hsa-miR-3960 |
| hsa-miR-337-3p | hsa-miR-374c-3p | hsa-miR-3613-3p | hsa-miR-485-3p |
| hsa-let-7d-5p | hsa-miR-425-5p | hsa-miR-655-3p | hsa-miR-6087 |
| hsa-miR-145-5p | hsa-miR-181a-5p | hsa-miR-7-1-3p | hsa-miR-92a-1-5p |
| hsa-miR-93-5p | hsa-miR-196b-5p | hsa-miR-23a-5p | hsa-miR-4668-5p |
| hsa-miR-619-5p | hsa-let-7f-1-3p | hsa-miR-24-2-5p | hsa-miR-3605-3p |
| hsa-miR-130a-3p | | | |

In certain embodiments, at least three miRNAs are selected in a group comprising or consisting of hsa-let-7a-5p, hsa-let-7i-5p, hsa-miR-660-5p, hsa-miR-6832-3p, hsa-let-7b-5p, hsa-miR-409-3p, hsa-miR-664a-3p, hsa-miR-146a-5p, hsa-miR-24-3p, hsa-miR-210-3p, hsa-miR-185-5p, hsa-miR-16-2-3p, hsa-miR-21-5p, hsa-miR-199a-3p, hsa-miR-3651, hsa-miR-18 lb-5p, hsa-let-7f-5p, hsa-miR-30a-3p, hsa-miR-495-3p, hsa-miR-26a-2-3p, hsa-miR-574-3p, hsa-miR-320b, hsa-let-7a-3p, hsa-miR-376a-3p, hsa-miR-23b-3p, hsa-miR-193a-5p, hsa-miR-28-5p, hsa-miR-539-5p, hsa-miR-1273g-3p, hsa-miR-382-5p, hsa-miR-99b-3p, hsa-miR-708-5p, hsa-miR-25-3p, hsa-miR-423-3p, hsa-miR-103a-3p, hsa-miR-98-3p, hsa-miR-199a-5p, hsa-miR-17-5p, hsa-miR-19a-3p, hsa-miR-1237-5p, hsa-miR-196a-5p, hsa-miR-19b-3p, hsa-miR-126-5p, hsa-miR-223-3p, hsa-miR-214-3p, hsa-miR-92b-3p, hsa-miR-2053, hsa-miR-532-5p, hsa-miR-125a-5p, hsa-miR-320a, hsa-miR-29b-1-5p, hsa-miR-542-3p, hsa-miR-221-3p, hsa-miR-3074-5p, hsa-miR-3648, hsa-miR-663a, hsa-miR-222-3p, hsa-miR-376c-3p, hsa-miR-374a-3p, hsa-miR-101-3p, hsa-let-7e-5p, hsa-let-7b-3p, hsa-miR-454-3p, hsa-miR-143-3p, hsa-miR-191-5p, hsa-miR-625-3p, hsa-miR-532-3p, hsa-miR-21-3p, hsa-miR-199b-3p, hsa-miR-99b-5p, hsa-miR-136-3p, hsa-miR-224-5p, hsa-miR-342-3p, hsa-miR-34a-5p, hsa-miR-361-3p, hsa-miR-26a-5p, hsa-miR-23a-3p, hsa-miR-5096, hsa-miR-1246, hsa-miR-27a-5p, hsa-miR-424-3p, hsa-miR-30e-3p, hsa-miR-130b-3p, hsa-miR-324-5p, hsa-miR-28-3p, hsa-miR-22-3p, hsa-miR-134-5p, hsa-miR-340-3p, hsa-let-7g-5p, hsa-miR-151a-3p, hsa-miR-154-5p, hsa-miR-379-5p, hsa-miR-92a-3p, hsa-miR-186-5p, hsa-miR-34a-3p, hsa-miR-409-5p, hsa-miR-424-5p, hsa-miR-193b-5p, hsa-miR-576-5p, hsa-miR-543, hsa-let-7d-3p, hsa-miR-328-3p, hsa-miR-874-3p, hsa-miR-5787, hsa-miR-4454, hsa-miR-4449, hsa-miR-100-5p, hsa-miR-6089, hsa-miR-146b-5p, hsa-miR-27a-3p, hsa-miR-103b, hsa-miR-127-3p, hsa-miR-423-5p, hsa-miR-30c-5p, hsa-miR-1273a, hsa-miR-149-5p, hsa-miR-29a-3p, hsa-miR-494-3p, hsa-miR-1306-5p, hsa-miR-181c-5p, hsa-miR-574-5p, hsa-miR-98-5p, hsa-miR-138-5p, hsa-miR-193b-3p, hsa-miR-199b-5p, hsa-miR-10a-5p, hsa-miR-15b-3p, hsa-miR-222-5p, hsa-miR-125b-5p, hsa-miR-29b-3p, hsa-miR-26b-3p, hsa-miR-3613-5p, hsa-miR-3184-3p, hsa-miR-374b-5p, hsa-miR-10b-5p, hsa-miR-365b-3p, hsa-let-7c-5p, hsa-miR-335-5p, hsa-miR-22-5p, hsa-miR-3960, hsa-miR-337-3p, hsa-miR-374c-3p, hsa-miR-3613-3p, hsa-miR-485-3p, hsa-let-7d-5p, hsa-miR-425-5p, hsa-miR-655-3p, hsa-miR-6087, hsa-miR-145-5p, hsa-miR-181a-5p, hsa-miR-7-1-3p, hsa-miR-92a-1-5p, hsa-miR-93-5p, hsa-miR-196b-5p, hsa-miR-23a-5p, hsa-miR-4668-5p, hsa-miR-619-5p, hsa-let-7f-1-3p, hsa-miR-24-2-5p, hsa-miR-3605-3p, hsa-miR-130a-3p and a combination thereof.

TABLE 5
miRNAs according to the invention

| | | | |
|---|---|---|---|
| hsa-let-7a-5p | hsa-miR-210-3p | hsa-miR-29b-3p | hsa-miR-30e-3p |
| hsa-let-7b-5p | hsa-miR-3184-3p | hsa-miR-92a-3p | hsa-miR-320a |
| hsa-miR-24-3p | hsa-let-7d-5p | hsa-miR-193b-5p | hsa-miR-361-3p |
| hsa-miR-199a-5p | hsa-miR-25-3p | hsa-miR-181a-5p | hsa-miR-151a-3p |
| hsa-miR-214-3p | hsa-miR-193a-5p | hsa-miR-30c-5p | hsa-miR-154-5p |
| hsa-let-7f-5p | hsa-miR-199a-3p | hsa-miR-664b-3p | hsa-miR-664a-5p |
| hsa-miR-3607-5p | hsa-miR-29a-3p | hsa-miR-27a-3p | hsa-miR-92b-3p |
| hsa-miR-199b-3p | hsa-miR-342-3p | hsa-miR-320b | hsa-miR-1291 |
| hsa-let-7e-5p | hsa-miR-130a-3p | hsa-miR-3651 | hsa-miR-103b |
| hsa-miR-1273g-3p | hsa-miR-30a-3p | hsa-miR-664b-5p | hsa-miR-34a-3p |
| hsa-miR-125a-5p | hsa-miR-145-5p | hsa-miR-664a-3p | hsa-miR-140-5p |
| hsa-miR-21-5p | hsa-miR-28-3p | hsa-miR-98-5p | hsa-miR-3609 |
| hsa-let-7i-5p | hsa-miR-93-5p | hsa-miR-146b-5p | hsa-miR-374c-3p |
| hsa-miR-125b-5p | hsa-miR-34a-5p | hsa-miR-337-3p | hsa-miR-10a-5p |
| hsa-let-7g-5p | hsa-miR-222-3p | hsa-miR-4449 | hsa-miR-22-3p |
| hsa-miR-191-5p | hsa-miR-3074-5p | hsa-miR-6516-3p | hsa-miR-4668-5p |
| hsa-miR-574-3p | hsa-miR-424-5p | hsa-let-7i-3p | hsa-miR-24-2-5p |
| hsa-miR-199b-5p | hsa-miR-424-3p | hsa-miR-103a-3p | hsa-miR-29b-1-5p |
| hsa-miR-423-5p | hsa-miR-328-3p | hsa-miR-324-5p | hsa-miR-335-5p |
| hsa-miR-574-5p | hsa-miR-17-5p | hsa-miR-660-5p | hsa-miR-425-5p |
| hsa-miR-23b-3p | hsa-miR-23a-3p | hsa-miR-185-5p | hsa-miR-4461 |
| hsa-miR-196a-5p | hsa-let-7d-3p | hsa-miR-374b-5p | hsa-miR-127-3p |
| hsa-let-7c-5p | hsa-miR-423-3p | hsa-miR-409-3p | hsa-miR-196b-5p |
| hsa-miR-221-3p | hsa-miR-382-5p | hsa-miR-619-5p | hsa-miR-3613-5p |
| hsa-miR-3653-5p | hsa-miR-19b-3p | hsa-miR-99b-5p | hsa-miR-376c-3p |
| hsa-miR-99b-3p | hsa-miR-663b | hsa-miR-495-3p | hsa-miR-454-3p |

In some embodiments, the at least three miRNAs are selected in a group comprising hsa-let-7a-5p, hsa-miR-210-3p, hsa-miR-29b-3p, hsa-miR-30e-3p, hsa-let-7b-5p, hsa-miR-3184-3p, hsa-miR-92a-3p, hsa-miR-320a, hsa-miR-24-3p, hsa-let-7d-5p, hsa-miR-193b-5p, hsa-miR-361-3p, hsa-miR-199a-5p, hsa-miR-25-3p, hsa-miR-181a-5p, hsa-miR-151a-3p, hsa-miR-214-3p, hsa-miR-193a-5p, hsa-miR-30c-5p, hsa-miR-154-5p, hsa-let-7f-5p, hsa-miR-199a-3p, hsa-miR-664b-3p, hsa-miR-664a-5p, hsa-miR-3607-5p, hsa-miR-29a-3p, hsa-miR-27a-3p, hsa-miR-92b-3p, hsa-miR-199b-3p, hsa-miR-342-3p, hsa-miR-320b, hsa-miR-1291, hsa-let-7e-5p, hsa-miR-130a-3p, hsa-miR-3651, hsa-miR-103b, hsa-miR-1273g-3p, hsa-miR-30a-3p, hsa-miR-664b-5p, hsa-miR-34a-3p, hsa-miR-125a-5p, hsa-miR-145-5p, hsa-miR-664a-3p, hsa-miR-140-5p, hsa-miR-21-5p, hsa-miR-28-3p, hsa-miR-98-5p, hsa-miR-3609, hsa-let-7i-5p, hsa-miR-93-5p, hsa-miR-146b-5p, hsa-miR-374c-3p, hsa-miR-125b-5p, hsa-miR-34a-5p, hsa-miR-337-3p, hsa-miR-10a-5p, hsa-let-7g-5p, hsa-miR-222-3p, hsa-miR-4449, hsa-miR-22-3p, hsa-miR-191-5p, hsa-miR-3074-5p, hsa-miR-6516-3p, hsa-miR-4668-5p, hsa-miR-574-3p, hsa-miR-424-5p, hsa-let-7i-3p, hsa-miR-24-2-5p, hsa-miR-199b-5p, hsa-miR-424-3p, hsa-miR-103a-3p, hsa-miR-29b-1-5p, hsa-miR-423-5p, hsa-miR-328-3p, hsa-miR-324-5p, hsa-miR-335-5p, hsa-miR-574-5p, hsa-miR-17-5p, hsa-miR-660-5p, hsa-miR-425-5p, hsa-miR-23b-3p, hsa-miR-23a-3p, hsa-miR-185-5p, hsa-miR-4461, hsa-miR-196a-5p, hsa-let-7d-3p, hsa-miR-374b-5p, hsa-miR-127-3p, hsa-let-7c-5p, hsa-miR-423-3p, hsa-miR-409-3p, hsa-miR-196b-5p, hsa-miR-221-3p, hsa-miR-382-5p, hsa-miR-619-5p, hsa-miR-3613-5p, hsa-miR-3653-5p, hsa-miR-19b-3p, hsa-miR-99b-5p, hsa-miR-376c-3p, hsa-miR-99b-3p, hsa-miR-663b, hsa-miR-495-3p, hsa-miR-454-3p, and a combination thereof.

TABLE 6
miRNAs according to the invention

| | | | |
|---|---|---|---|
| hsa-let-7a-5p | hsa-miR-3653-5p | hsa-miR-98-5p | hsa-miR-28-5p |
| hsa-let-7b-5p | hsa-miR-342-3p | hsa-miR-664a-3p | hsa-miR-10a-5p |
| hsa-miR-24-3p | hsa-miR-28-3p | hsa-miR-92b-3p | hsa-miR-151a-3p |
| hsa-let-7f-5p | hsa-miR-23b-3p | hsa-miR-4449 | hsa-miR-30e-3p |
| hsa-miR-199a-5p | hsa-let-7c-5p | hsa-miR-320a | hsa-miR-324-5p |
| hsa-miR-214-3p | hsa-miR-222-3p | hsa-miR-181a-5p | hsa-miR-495-3p |
| hsa-miR-3607-5p | hsa-miR-29a-3p | hsa-miR-3651 | hsa-miR-576-5p |
| hsa-miR-125a-5p | hsa-miR-92a-3p | hsa-miR-185-5p | hsa-miR-625-3p |
| hsa-miR-199b-3p | hsa-miR-30a-3p | hsa-miR-664b-5p | hsa-miR-671-5p |
| hsa-miR-125b-5p | hsa-miR-424-3p | hsa-miR-196b-5p | hsa-miR-1271-5p |
| hsa-miR-21-5p | hsa-miR-423-3p | hsa-miR-27a-3p | hsa-miR-186-5p |
| hsa-let-7e-5p | hsa-miR-34a-5p | hsa-miR-29b-3p | hsa-miR-23a-5p |
| hsa-let-7i-5p | hsa-miR-424-5p | hsa-miR-664b-3p | hsa-miR-3613-5p |
| hsa-let-7g-5p | hsa-miR-145-5p | hsa-miR-99b-5p | hsa-miR-376c-3p |
| hsa-miR-574-3p | hsa-miR-328-3p | hsa-miR-103a-3p | hsa-miR-409-3p |
| hsa-miR-574-5p | hsa-miR-3074-5p | hsa-miR-6516-3p | hsa-miR-4461 |
| hsa-miR-191-5p | hsa-let-7d-3p | hsa-miR-22-3p | hsa-miR-454-3p |
| hsa-miR-196a-5p | hsa-miR-93-5p | hsa-miR-26a-5p | hsa-miR-6724-5p |
| hsa-miR-221-3p | hsa-miR-23a-3p | hsa-miR-103b | hsa-let-7b-3p |
| hsa-miR-25-3p | hsa-miR-19b-3p | hsa-miR-1291 | hsa-miR-190a-5p |
| hsa-miR-423-5p | hsa-miR-146b-5p | hsa-miR-425-5p | hsa-miR-26b-3p |
| hsa-miR-210-3p | hsa-miR-320b | hsa-miR-22-5p | hsa-miR-3609 |
| hsa-miR-1273g-3p | hsa-miR-337-3p | hsa-miR-374c-3p | hsa-miR-411-5p |
| hsa-let-7d-5p | hsa-miR-17-5p | hsa-let-7i-3p | hsa-miR-425-3p |
| hsa-miR-199b-5p | hsa-miR-130a-3p | hsa-miR-374b-5p | hsa-miR-4485-3p |
| hsa-miR-199a-3p | hsa-miR-193b-5p | hsa-miR-455-3p | hsa-miR-30c-5p |
| hsa-miR-193a-5p | hsa-miR-382-5p | hsa-miR-532-3p | hsa-miR-619-5p |
| hsa-miR-3184-3p | | | |

In some embodiments, at least three miRNAs are selected in a group comprising or consisting of hsa-let-7a-5p, hsa-miR-3653-5p, hsa-miR-98-5p, hsa-miR-28-5p, hsa-let-7b-5p, hsa-miR-342-3p, hsa-miR-664a-3p, hsa-miR-10a-5p, hsa-miR-24-3p, hsa-miR-28-3p, hsa-miR-92b-3p, hsa-miR-151a-3p, hsa-let-7f-5p, hsa-miR-23b-3p, hsa-miR-4449, hsa-miR-30e-3p, hsa-miR-199a-5p, hsa-let-7c-5p, hsa-miR-320a, hsa-miR-324-5p, hsa-miR-214-3p, hsa-miR-222-3p, hsa-miR-181a-5p, hsa-miR-495-3p, hsa-miR-3607-5p, hsa-miR-29a-3p, hsa-miR-3651, hsa-miR-576-5p, hsa-miR-125a-5p, hsa-miR-92a-3p, hsa-miR-185-5p, hsa-miR-625-3p, hsa-miR-199b-3p, hsa-miR-30a-3p, hsa-miR-664b-5p, hsa-miR-671-5p, hsa-miR-125b-5p, hsa-miR-424-3p, hsa-miR-196b-5p, hsa-miR-1271-5p, hsa-miR-21-5p, hsa-miR-423-3p, hsa-miR-27a-3p, hsa-miR-186-5p, hsa-let-7e-5p, hsa-miR-34a-5p, hsa-miR-29b-3p, hsa-miR-23a-5p, hsa-let-7i-5p, hsa-miR-424-5p, hsa-miR-664b-3p, hsa-miR-3613-5p, hsa-let-7g-5p, hsa-miR-145-5p, hsa-miR-99b-5p, hsa-miR-376c-3p, hsa-miR-574-3p, hsa-miR-328-3p, hsa-miR-103a-3p, hsa-miR-409-3p, hsa-miR-574-5p, hsa-miR-3074-5p, hsa-miR-6516-3p, hsa-miR-4461, hsa-miR-191-5p, hsa-let-7d-3p, hsa-miR-22-3p, hsa-miR-454-3p, hsa-miR-196a-5p, hsa-miR-93-5p, hsa-miR-26a-5p, hsa-miR-6724-5p, hsa-miR-221-3p, hsa-miR-23a-3p, hsa-miR-103b, hsa-let-7b-3p, hsa-miR-25-3p, hsa-miR-19b-3p, hsa-miR-1291, hsa-miR-190a-5p, hsa-miR-423-5p, hsa-miR-146b-5p, hsa-miR-425-5p, hsa-miR-26b-3p, hsa-miR-210-3p, hsa-miR-320b, hsa-miR-22-5p, hsa-miR-3609, hsa-miR-1273g-3p, hsa-miR-337-3p, hsa-miR-374c-3p, hsa-miR-411-5p, hsa-let-7d-5p, hsa-miR-17-5p, hsa-let-7i-3p, hsa-miR-425-3p, hsa-miR-199b-5p, hsa-miR-130a-3p, hsa-miR-374b-5p, hsa-miR-4485-3p, hsa-miR-199a-3p, hsa-miR-193b-5p, hsa-miR-455-3p, hsa-miR-30c-5p, hsa-miR-193a-5p, hsa-miR-382-5p, hsa-miR-532-3p, hsa-miR-619-5p, hsa-miR-3184-3p and a combination thereof.

TABLE 7
miRNAs according to the invention

| | | | |
|---|---|---|---|
| hsa-miR-3687 | hsa-miR-619-5p | hsa-let-7e-5p | hsa-miR-24-3p |
| hsa-miR-664b-5p | hsa-miR-181a-5p | hsa-miR-25-3p | hsa-miR-382-5p |
| hsa-miR-210-3p | hsa-miR-409-3p | hsa-miR-374c-3p | hsa-miR-214-3p |
| hsa-miR-4449 | hsa-let-7a-3p | hsa-miR-29b-3p | hsa-miR-199b-5p |
| hsa-miR-3651 | hsa-miR-4454 | hsa-let-7b-3p | hsa-miR-199a-5p |
| hsa-miR-663a | hsa-let-7i-5p | hsa-miR-23b-3p | hsa-miR-3074-5p |
| hsa-miR-664b-3p | hsa-miR-335-5p | hsa-miR-3613-3p | hsa-miR-361-3p |
| hsa-miR-3653-5p | hsa-miR-1246 | hsa-miR-138-5p | hsa-miR-6723-5p |
| hsa-miR-664a-3p | hsa-miR-6516-5p | hsa-miR-6516-3p | hsa-miR-130a-3p |
| hsa-miR-3648 | hsa-miR-3607-5p | hsa-miR-4485-3p | hsa-miR-660-5p |
| hsa-miR-196b-5p | hsa-miR-342-3p | hsa-miR-221-3p | |

In some embodiments, the at least three miRNAs are selected in a group comprising hsa-miR-3687, hsa-miR-619-5p, hsa-let-7e-5p, hsa-miR-24-3p, hsa-miR-664b-5p, hsa-miR-181a-5p, hsa-miR-25-3p, hsa-miR-382-5p, hsa-miR-210-3p, hsa-miR-409-3p, hsa-miR-374c-3p, hsa-miR-214-3p, hsa-miR-4449, hsa-let-7a-3p, hsa-miR-29b-3p, hsa-miR-199b-5p, hsa-miR-3651, hsa-miR-4454, hsa-let-7b-3p, hsa-miR-199a-5p, hsa-miR-663a, hsa-let-7i-5p, hsa-miR-23b-3p, hsa-miR-3074-5p, hsa-miR-664b-3p, hsa-miR-335-5p, hsa-miR-3613-3p, hsa-miR-361-3p, hsa-miR-3653-5p, hsa-miR-1246, hsa-miR-138-5p, hsa-miR-6723-5p, hsa-miR-664a-3p, hsa-miR-6516-5p, hsa-miR-6516-3p, hsa-miR-130a-3p, hsa-miR-3648, hsa-miR-3607-5p, hsa-miR-4485-3p, hsa-miR-660-5p, hsa-miR-196b-5p, hsa-miR-342-3p, hsa-miR-221-3p, and a combination thereof.

TABLE 8
miRNAs according to the invention

| | | | |
|---|---|---|---|
| hsa-miR-210-3p | hsa-miR-409-3p | hsa-miR-219 | hsa-miR-29b |
| hsa-miR-4454 | hsa-miR-3607-5p | hsa-miR-299-5p | has-miR-140-5p |
| hsa-miR-619-5p | hsa-miR-3609 | hsa-miR-302b | hsa-miR-31 |
| hsa-miR-1246 | hsa-miR-663a | has-miR-221 | hsa-miR-30 |
| hsa-miR-222-3p | hsa-miR-19a-3p | hsa-miR-155 | hsa-miR-30e |
| hsa-miR-181a-5p | hsa-miR-3651 | hsa-miR-885-5p | hsa-miR-17 |
| hsa-miR-6832-3p | hsa-miR-4668-5p | hsa-miR-181a | hsa-miR-433 |
| hsa-miR-335-5p | hsa-miR-301a-3p | hsa-miR-320c | hsa-miR-486-5p |
| hsa-let-7a-3p | hsa-miR-664a-3p | hsa-miR-548d-5p | hsa-miR-335 |
| hsa-miR-28-3p | hsa-miR-485-5p | hsa-miR-34a | hsa-miR-106a |
| hsa-miR-125a-5p | hsa-miR-382-5p | hsa-miR-378 | hsa-miR-21-3p |
| hsa-miR-374c-3p | hsa-miR-4449 | hsa-346 | hsa-miR-26a-5p |
| hsa-miR-181c-5p | hsa-miR-138-5p | hsa-10a | let-7a-5p |
| hsa-miR-374b-5p | let-7a | hsa-125b | hsa-miR-10a |
| hsa-miR-3687 | hsa-miR-199b | hsa-miR-322 | hsa-miR-148-a |
| hsa-miR-3653-5p | hsa-miR-218 | hsa-miR-21 | hsa-miR-31-5p |
| hsa-miR-664b-5p | hsa-miR-148a | hsa-miR-96 | hsa-miR-486-5p |
| hsa-miR-664b-3p | hsa-miR-135b | hsa-miR-22 | hsa-miR-24-3p |
| hsa-miR-3613-3p | hsa-miR-203 | hsa-miR-27 | hsa-let-7i-5p |
| hsa-miR-3074-5p | hsa-miR-4485-3p | hsa-let-7c-5p | hsa-miR-6723-5p |
| hsa-miR-671-5p | hsa-miR-93-5p | hsa-miR-154-5p | |

In certain embodiments, at least three miRNAs are selected in a group comprising or consisting of hsa-miR-210-3p, hsa-miR-409-3p, hsa-miR-219, hsa-miR-29b, hsa-miR-4454, hsa-miR-3607-5p, hsa-miR-299-5p, has-miR-140-5p, hsa-miR-619-5p, hsa-miR-3609, hsa-miR-302b, hsa-miR-31, hsa-miR-1246, hsa-miR-663a, has-miR-221, hsa-miR-30, hsa-miR-222-3p, hsa-miR-19a-3p, hsa-miR-155, hsa-miR-30e, hsa-miR-181a-5p, hsa-miR-3651, hsa-miR-885-5p, hsa-miR-17, hsa-miR-6832-3p, hsa-miR-4668-5p, hsa-miR-181a, hsa-miR-433, hsa-miR-335-5p, hsa-miR-301a-3p, hsa-miR-320c, hsa-miR-486-5p, hsa-let-7a-3p, hsa-miR-664a-3p, hsa-miR-548d-5p, hsa-miR-335, hsa-miR-28-3p, hsa-miR-485-5p, hsa-miR-34a, hsa-miR-106a, hsa-miR-125a-5p, hsa-miR-382-5p, hsa-miR-378, hsa-miR-21-3p, hsa-miR-374c-3p, hsa-miR-4449, hsa-346, hsa-miR-26a-5p, hsa-miR-181c-5p, hsa-miR-138-5p, hsa-10a, let-7a-5p, hsa-miR-374b-5p, let-7a, hsa-125b, hsa-miR-10a, hsa-miR-3687, hsa-miR-199b, hsa-miR-322, hsa-miR-148-a, hsa-miR-3653-5p, hsa-miR-218, hsa-miR-21, hsa-miR-31-5p, hsa-miR-664b-5p, hsa-miR-148a, hsa-miR-96, hsa-miR-486-5p, hsa-miR-664b-3p, hsa-miR-135b, hsa-miR-22, hsa-miR-24-3p, hsa-miR-3613-3p, hsa-miR-203, hsa-miR-27, hsa-let-7i-5p, hsa-miR-3074-5p, hsa-miR-4485-3p, hsa-let-7c-5p, hsa-miR-6723-5p, hsa-miR-671-5p, hsa-miR-93-5p, hsa-miR-154-5p and a combination thereof.

TABLE 9
miRNAs according to the invention

| | | | |
|---|---|---|---|
| hsa-miR-210-3p | hsa-let-7i-5p | hsa-miR-29b-3p | hsa-miR-199a-5p |
| hsa-miR-619-5p | hsa-miR-335-5p | hsa-miR-23b-3p | hsa-miR-3074-5p |
| hsa-miR-181a-5p | hsa-miR-1246 | hsa-miR-24-3p | hsa-miR-361-3p |
| hsa-let-7a-3p | hsa-let-7e-5p | hsa-miR-214-3p | hsa-miR-130a-3p |
| hsa-miR-4454 | hsa-miR-374c-3p | hsa-miR-199b-5p | hsa-miR-3607-5p |
| hsa-miR-660-5p | hsa-miR-342-3p | | |

In some embodiments, the at least three miRNAs are selected in a group comprising hsa-miR-210-3p, hsa-let-7i-5p, hsa-miR-29b-3p, hsa-miR-199a-5p, hsa-miR-619-5p, hsa-miR-335-5p, hsa-miR-23b-3p, hsa-miR-3074-5p, hsa-miR-181a-5p, hsa-miR-1246, hsa-miR-24-3p, hsa-miR-361-3p, hsa-let-7a-3p, hsa-let-7e-5p, hsa-miR-214-3p, hsa-miR-130a-3p, hsa-miR-4454, hsa-miR-374c-3p, hsa-miR-199b-5p, hsa-miR-3607-5p, hsa-miR-660-5p, hsa-miR-342-3p, and a combination thereof.

TABLE 10
miRNAs according to the invention

| | | | |
|---|---|---|---|
| hsa-miR-210-3p | hsa-miR-125a-5p | hsa-miR-219 | hsa-miR-21 |
| hsa-miR-4454 | hsa-miR-374c-3p | hsa-miR-299-5p | hsa-miR-96 |
| hsa-miR-619-5p | hsa-miR-181c-5p | hsa-miR-302b | hsa-miR-22 |
| hsa-miR-1246 | hsa-miR-374b-5p | hsa-miR-548d-5p | hsa-miR-27 |
| hsa-miR-222-3p | let-7a | hsa-miR-34a | hsa-miR-29b |
| hsa-miR-181a-5p | hsa-miR-199b | hsa-miR-378 | hsa-miR-24-3p |
| hsa-miR-6832-3p | hsa-miR-218 | hsa-346 | hsa-let-7i-5p |
| hsa-miR-335-5p | hsa-miR-148a | hsa-10a | hsa-miR-3074-5p |
| hsa-let-7a-3p | hsa-miR-135b | hsa-125b | hsa-miR-671-5p |
| hsa-miR-28-3p | hsa-miR-203 | hsa-miR-322 | |

In certain embodiments, at least three miRNAs are selected in a group comprising or consisting of hsa-miR-210-3p, hsa-miR-125a-5p, hsa-miR-219, hsa-miR-21, hsa-miR-4454, hsa-miR-374c-3p, hsa-miR-299-5p, hsa-miR-96, hsa-miR-619-5p, hsa-miR-181c-5p, hsa-miR-302b, hsa-miR-22, hsa-miR-1246, hsa-miR-374b-5p, hsa-miR-548d-5p, hsa-miR-27, hsa-miR-222-3p, let-7a, hsa-miR-34a, hsa-miR-29b, hsa-miR-181a-5p, hsa-miR-199b, hsa-miR-378, hsa-miR-24-3p, hsa-miR-6832-3p, hsa-miR-218, hsa-346, hsa-let-7i-5p, hsa-miR-335-5p, hsa-miR-148a, hsa-10a, hsa-miR-3074-5p, hsa-let-7a-3p, hsa-miR-135b, hsa-125b, hsa-miR-671-5p, hsa-miR-28-3p, hsa-miR-203, hsa-miR-322 and a combination thereof.

TABLE 11
miRNAs according to the invention

| | | | |
|---|---|---|---|
| hsa-miR-3687 | hsa-miR-664b-3p | hsa-miR-6516-5p | hsa-miR-138-5p |
| hsa-miR-664b-5p | hsa-miR-3653-5p | hsa-miR-3607-5p | hsa-miR-6516-3p |
| hsa-miR-4449 | hsa-miR-664a-3p | hsa-miR-25-3p | hsa-miR-4485-3p |
| hsa-miR-3651 | hsa-miR-3648 | hsa-let-7b-3p | hsa-miR-382-5p |
| hsa-miR-663a | hsa-miR-409-3p | hsa-miR-3613-3p | hsa-miR-6723-5p |
| hsa-miR-3687 | hsa-miR-664b-3p | hsa-miR-6516-5p | hsa-miR-138-5p |
| hsa-miR-196b-5p | hsa-miR-221-3p | | |

In some embodiments, the at least three miRNAs are selected in a group comprising hsa-miR-3687, hsa-miR-664b-3p, hsa-miR-6516-5p, hsa-miR-138-5p, hsa-miR-664b-5p, hsa-miR-3653-5p, hsa-miR-3607-5p, hsa-miR-6516-3p, hsa-miR-4449, hsa-miR-664a-3p, hsa-miR-25-3p, hsa-miR-4485-3p, hsa-miR-3651, hsa-miR-3648, hsa-let-7b-3p, hsa-miR-382-5p, hsa-miR-663a, hsa-miR-409-3p, hsa-miR-3613-3p, hsa-miR-6723-5p, hsa-miR-3687, hsa-miR-664b-3p, hsa-miR-6516-5p, hsa-miR-138-5p, hsa-miR-196b-5p, hsa-miR-221-3p, and a combination thereof.

TABLE 12
miRNAs according to the invention

| | | | |
|---|---|---|---|
| hsa-miR-3687 | hsa-miR-19a-3p | has-miR-221 | hsa-miR-17 |
| hsa-miR-3653-5p | hsa-miR-3651 | hsa-miR-155 | hsa-miR-433 |
| hsa-miR-664b-5p | hsa-miR-4668-5p | hsa-miR-885-5p | hsa-miR-486-5p |
| hsa-miR-664b-3p | hsa-miR-301a-3p | hsa-miR-181a | hsa-miR-335 |
| hsa-miR-3613-3p | hsa-miR-664a-3p | hsa-miR-320c | hsa-miR-106a |
| hsa-miR-409-3p | hsa-miR-485-5p | has-miR-140-5p | hsa-miR-4485-3p |
| hsa-miR-3607-5p | hsa-miR-382-5p | hsa-miR-31 | hsa-miR-93-5p |
| hsa-miR-3609 | hsa-miR-4449 | hsa-miR-30 | hsa-let-7c-5p |
| hsa-miR-663a | hsa-miR-138-5p | hsa-miR-30e | hsa-miR-154-5p |
| hsa-miR-6723-5p | | | |

In certain embodiments, at least three miRNAs are selected in a group comprising or consisting of hsa-miR-3687, hsa-miR-19a-3p, has-miR-221, hsa-miR-17, hsa-miR-3653-5p, hsa-miR-3651, hsa-miR-155, hsa-miR-433, hsa-miR-664b-5p, hsa-miR-4668-5p, hsa-miR-885-5p, hsa-miR-486-5p, hsa-miR-664b-3p, hsa-miR-301a-3p, hsa-miR-181a, hsa-miR-335, hsa-miR-3613-3p, hsa-miR-664a-3p, hsa-miR-320c, hsa-miR-106a, hsa-miR-409-3p, hsa-miR-485-5p, has-miR-140-5p, hsa-miR-4485-3p, hsa-miR-3607-5p, hsa-miR-382-5p, hsa-miR-31, hsa-miR-93-5p, hsa-miR-3609, hsa-miR-4449, hsa-miR-30, hsa-let-7c-5p, hsa-miR-663a, hsa-miR-138-5p, hsa-miR-30e, hsa-miR-154-5p, hsa-miR-6723-5p and a combination thereof.

In certain embodiments, the at least three miRNAs are selected in a group comprising hsa-miR-210-3p, hsa-miR-409-3p, hsa-miR-361-3p, hsa-miR-130a-3p, hsa-miR-660-5p, hsa-miR-199b-5p, hsa-miR-3074-5p, hsa-let-7i-5p, hsa-miR-24-3p, hsa-miR-342-3p, hsa-miR-214-3p, hsa-miR-199a-5p, hsa-miR-3607-5p, hsa-miR-221-3p, hsa-miR-4449, hsa-miR-382-5p, hsa-miR-196b-5p, hsa-miR-663a, hsa-miR-4485-3p, hsa-miR-6723-5p and a combination thereof.

In some embodiments, the at least three miRNAs are selected in a group comprising hsa-miR-210-3p, hsa-miR-409-3p, hsa-let-7i-5p, hsa-miR-3607-5p, hsa-let-7a-3p, hsa-miR-1246, hsa-miR-335-5p, hsa-miR-4454, hsa-miR-181a-5p, hsa-miR-374c-3p, hsa-miR-619-5p, hsa-miR-29b-3p, hsa-let7e-5p, hsa-miR-23b-3p, hsa-miR-4449, hsa-miR-663a, hsa-miR-25-3p, hsa-let-7b-3p, hsa-miR-138-5p, hsa-miR-3613-3p, hsa-miR-6516-3p, hsa-miR-664a-3p, hsa-miR-3648, hsa-miR-3653-5p, hsa-miR-6516-5p, hsa-miR-3651, hsa-miR-3687, hsa-miR-664-5p, hsa-miR-664-3p, and a combination thereof.

In certain embodiments, the at least three miRNAs are selected in a group comprising hsa-miR-210-3p, hsa-miR-361-3p, hsa-miR-130a-3p, hsa-miR-660-5p, hsa-miR-199b-5p, hsa-miR-3074-5p, hsa-let-7i-5p, hsa-miR-24-3p, hsa-miR-342-3p, hsa-miR-214-3p, hsa-miR-199a-5p, hsa-let-7a-3p, hsa-miR-1246, hsa-miR-335-5p, hsa-miR-4454, hsa-miR-181a-5p, hsa-miR-374c-3p, hsa-miR-619-5p, hsa-miR-29b-3p, hsa-let7e-5p, hsa-miR-23b-3p, and a combination thereof.

In some embodiments, the at least three miRNAs are selected in a group comprising miR-210-3p, hsa-miR-409-3p, hsa-let-7i-5p, hsa-miR-3607-5p, hsa-miR-4449, hsa-miR-663a, and a combination thereof.

In some embodiments, said at least three miRNAs are selected in a group comprising hsa-miR210-3p, hsa-miR-409-3p, hsa-let-7i-5p, hsa-miR-24-3p, hsa-miR-93-5p, hsa-miR-382-5p, hsa-miR-4485-3p, and a combination thereof.

In some embodiments, said at least three miRNA are selected in a group comprising hsa-miR210-3p, hsa-miR-409-3p, hsa-miR-4454, hsa-miR-619-5p, hsa-miR-3607-5p, hsa-miR-3613-3p, hsa-miR-664b-5p, hsa-miR-3687, hsa-miR-3653-5p, hsa-miR-664b-3p, and a combination thereof.

In certain embodiments, said at least three miRNAs are selected in a group comprising hsa-miR210-3p, hsa-let-7i-5p, hsa-miR-24-3p, hsa-miR-4454, hsa-miR-619-5p, and a combination thereof.

In certain embodiments, said at least three miRNAs are selected in a group comprising hsa-miR-210-3p, hsa-miR-409-3p, hsa-let-7i-5p, hsa-miR-24-3p, hsa-miR-382-5p, hsa-miR-4485-3p, and a combination thereof.

In some embodiments, said at least three miRNAs are selected in a group comprising hsa-miR210-3p, hsa-miR-409-3p, hsa-miR-4454, hsa-miR-619-5p, hsa-miR-3607-5p, hsa-miR-3613-3p, hsa-miR-664b-5p, hsa-miR-3687, hsa-miR-3653-5p, hsa-miR-664b-3p, and a combination thereof.

In some embodiments, said at least three miRNAs comprise hsa-miR210-3p and/or hsa-miR-409-3p.

In certain embodiments, said at least three miRNAs comprise hsa-miR210-3p. In some embodiments, said at least three miRNAs comprise hsa-miR-409-3p.

In another embodiment, the pharmaceutical composition of the invention comprises a therapeutically effective amount of at least 4, 5, 6, 7, 8, 9, 10, 12, 14, 16, 18, 20 miRNAs selected in any one of Table 1, Table 2, Table 3, Table 4, Table 5, Table 6, Table 7, Table 8, Table 9, Table 10, Table 11 or Table 12.

In some embodiments, said composition comprises a combination of hsa-miR-210-3p, hsa-miR-361-3p, hsa-miR-130a-3p, hsa-miR-660-5p, hsa-miR-199b-5p, hsa-miR-3074-5p, hsa-let-7i-5p, hsa-miR-24-3p, hsa-miR-342-3p, hsa-miR-214-3p, hsa-miR-199a-5p, hsa-miR-3607-5p.

In certain embodiments, the composition comprises a combination of hsa-miR210-3p, hsa-let-7i-5p, hsa-miR-24-3p.

In some embodiments, said composition comprises a combination of hsa-miR210-3p, hsa-miR-409-3p, hsa-let-7i-5p, hsa-miR-24-3p, hsa-miR-93-5p, hsa-miR-382-5p, and hsa-miR-4485-3p.

In some preferred embodiments, said composition comprises a combination of hsa-miR210-3p, hsa-let-7i-5p, hsa-miR-24-3p, hsa-miR-93-5p and hsa-miR-382-5p. In some preferred embodiments, said composition comprises at least hsa-miR210-3p.

In some embodiments, the at least three miRNAs are the active agents. In certain embodiments, the at least three miRNAs are the sole active agents in the pharmaceutical composition. As used herein, the expression “sole active agents” means that the at least three miRNAs represent the only or exclusive active ingredients for preventing and/or treating a tissue disorder, including a skin disorder, a bone disorder and/or a cartilage disorder. In another embodiment, the composition comprises one or more other active agent(s) than the miRNAs of the invention.

In some embodiments, the miRNAs may be synthesized by suitable cells, preferably cells having undergone tissue-differentiation, more preferably differentiated cells having tissue regenerating and/or repairing properties. As used herein, the expression “differentiated cells having tissue regenerating and/or repairing properties” is intended to refer to a cell population that possesses the ability to promote tissue regenerating and/or repairing, and/or to maintain existing tissues in a healthy physiological condition.

In one embodiment, the cells have undergone osteogenic, chondrogenic, epithelial, endothelial, myogenic or adipogenic differentiation. In some embodiments, the cells have undergone osteogenic and/or chondrogenic differentiation. In certain embodiments, the cells have undergone epithelial, endothelial, myogenic or adipogenic differentiation.

In some embodiments, said differentiated cells are selected in a group comprising primary cells, stem cells, genetically modified cells, and a combination thereof.

In some embodiments, primary cells may be selected in a group comprising or consisting of osteocytes, osteoblasts, osteoclasts, chondroblasts, chondrocytes, keratinocytes, dermal fibroblasts, fibroblasts, epithelial cells, hematopoietic cells, hepatic cells, neural cells, myofibroblasts, epithelial cells, endothelial cells, connective cells, adipocytes, and a combination thereof, and precursors thereof.

In certain embodiments, said cells are selected in a group comprising primary cells, in particular selected in a group comprising or consisting of osteocytes, osteoblasts, osteoclasts, chondroblasts, chondrocytes and a mixture thereof; stem cells, in particular selected in a group comprising or consisting of osteoprogenitors, embryonic stem cells (ESCs), mesenchymal stem cells (MSCs), pluripotent stem cells (pSCs), induced pluripotent stem cells (ipSCs) and a mixture thereof, genetically modified cells; and a combination thereof.

In some embodiments, the miRNAs synthesized by the cells may be extracted from the cells and/or may be recovered from exosomes or exosome-like vesicles that are secreted by the cells.

In practice, the RNAs content of the cells according to the instant invention may be assessed by any suitable method known in the art, or any method adapted therefrom. Illustratively, RNA may be extracted, e.g., by the mean of commercial kit (such as miRNeasy kit from Qiagen®); and further sequenced, e.g., by the mean of a high-throughput sequencing system (such as NextSeq 500 system from Illumina®).

Illustratively, one may use the Qiazol lysis reagent (Qiagene, Hilden, Germany) and a Precellys homogenizer (Bertine instruments, Montigny-le-Bretonneux, France). RNAs may be purified using Rneasy mini kit (Qiagen®, Hilden, Germany) with an additional on column DNase digestion according to the manufacturer's instruction. Quality and quantity of RNA may be determined using a spectrophotometer (Spectramax 190, Molecular Devices, California, USA). cDNA may be synthesized from 0.5 μg of total RNA using RT² RNA first strand kit (Qiagene, Hilden, Germany) for genes expression profiles though customized PCR arrays (Customized Human Osteogenic and angiogenic RT² Profiler Assay—Qiagen®, Hilden, Germany). The ABI Quantstudio 5 system (Applied Biosystems®) and SYBR Green ROX Mastermix (Qiagen®, Hilden, Germany) may be used for detection of the amplification product. Quantification may be obtained according to the ΔΔCT method. The final result of each sample may be normalized to the means of expression level of three housekeeping genes (e.g., ACTB, B2M and GAPDH). In practice, cellular miRNAs may be isolated, i.e., recovered from cells, by any suitable method known from the state of the art, or a method adapted therefrom. One may refer to, e.g., Chapter 7: Extraction, Purification, and Analysis of mRNA from Eukaryotic Cells of Molecular Cloning: a laboratory manual (Russell and Sambrook; 2001; Cold Spring Harbor Laboratory). Illustratively, miRNAs may be isolated by a commercial kit, such as, e.g., RNeasy Mini kit (Qiagen®) or MagMax mirVana Total RNA isolation kit (Applied Biosystems®), miRNeasy kit Mastermix (Qiagen®, Hilden, Germany), following the manufacturer's instructions. RNA concentration may be determined by Nanodrop (ThermoFisher®, Waltham, Mass., USA).

In some embodiments, the miRNAs may be synthesized de novo, by any suitable method known in the state of the art, or a method adapted therefrom. In some embodiments, the miRNAs are in vitro and/or in vivo synthesized.

In some embodiments, the composition is desiccated and/or sterilized.

In certain embodiments, the composition is desiccated. As used herein, the term “desiccated” and the term “dehydrated” are intended to be substituted by one another.

In some embodiments, desiccation is obtained by freeze-drying. Illustratively, freeze-drying, otherwise referred to as lyophilization, may be performed accordingly any one of the protocols disclosed in the state of the art, or a protocol adapted therefrom. In some embodiments, the freeze-drying of the composition is performed at a temperature of about −80° C., under vacuum.

In certain embodiments, sterilization is obtained by gamma-irradiation, preferably at a dose of about 7 kGy to about 45 kGy, more preferably at room temperature.

Within the scope of the invention, the expression “about 7 kGy to about 45 KGy” encompasses 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 and 45 kGy.

In some embodiments, the sterilization is obtained by gamma-irradiation at a dose of about 10 kGy to about 40 kGy.

Within the scope of the invention, the term “room temperature” is intended to refer to a temperature comprised from about 18° C. to about 22° C., which encompasses 18° C., 19° C., 20° C., 21° C. and 22° C. In some embodiments, room temperature is a temperature of about 20° C.

In some embodiments, the gamma-irradiation may be performed at a temperature below about 10° C., preferably on ice (about 0° C.). Within the scope of the invention, a temperature below about 10° C. encompasses 9.5° C., 8° C., 8.5° C., 8° C., 7.5° C., 7° C., 6.5° C., 6° C., 5° C., 4° C., 3° C., 2° C., 1° C., 0° C., −1° C., −2° C., −3° C., −4° C., −5° C., −10° C., −20° C., −30° C., −40° C., −50° C., −60° C., −70° C. and −80° C.

In practice, the gamma-irradiation may be performed for a duration that would depend from the amount (e.g., expressed in ng, μg, mg or g) of ingredients to be sterilized and/or the dose to be administered. In certain embodiments, the gamma-irradiation may be performed from about 10 sec to about 24 h, preferably from about 5 min (300 sec) to about 12h, more preferably, from about 10 min (600 sec) to about 3 h (10,800 sec). Within the scope of the invention, the expression “from about 10 sec to about 24 h” encompasses 10 sec, 12 sec, 14 sec, 16 sec, 18 sec 20 sec, 25 sec, 30 sec, 35 sec, 40 sec, 45 sec, 50 sec, 55 sec, 1 min, 1 min 30, 2 min, 2 min 30, 3 min, 3 min 30, 4 min, 4 min 30, 5 min, 6 min, 7 min, 8 min, 9 min, 10 min, 12 min, 14 min, 16 min, 18 min, 20 min, 22 min, 24 min, 26 min, 28 min, 30 min, 35 min, 40 min, 45 min, 50 min, 55 min, 1 h, 1 h 30, 2 h, 2 h 30, 3 h, 3 h 30, 4 h, 4 h 30, 5 h, 5 h 30, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h and 24 h.

As used herein, “pharmaceutically acceptable vehicle” refers to any solvent, dispersion medium, coating, antibacterial and/or antifungal agent, isotonic and absorption delaying agent and the like.

In one embodiment, the pharmaceutical composition comprises one or more pharmaceutically acceptable vehicle, such as emulsifiers, viscosity increasing agents, antimicrobial agents, antioxidants, preservatives, gelling agents, permeation enhancers or stabilizing agents.

In practice, the pharmaceutically acceptable vehicle may comprise one or more ingredient(s) selected in a group of additives polypeptides; amino acids; lipids; and carbohydrates. Among carbohydrates, one may cite sugars, including monosaccharides, di-, tri-, tetra-, and oligosaccharides; derivatized sugars such as alditols, aldonic acids, esterified sugars and the like; and polysaccharides or sugar polymers.

Examples of suitable pharmaceutically acceptable vehicles may include polypeptides such as, e.g., gelatin, casein, and the like.

Another aspect of the invention relates to a medical device comprising a pharmaceutical composition according to the invention.

In some embodiments, the medical device is an implant. In some embodiments, the implant may be in the form of an organic or inorganic scaffold. In certain embodiments, the implant is resorbable.

The invention further pertains to an implant comprising a pharmaceutical composition according to the instant disclosure. In some embodiments, the implant is allogeneic. In certain embodiments, the implant is autologous. In some embodiments, the implant is xenogeneic. In certain embodiments, the implant is lyophilized and sterilized, preferably sterilized by gamma-irradiation.

In certain embodiments, the medical device is a dressing for local application. In some embodiments, the dressing may comprise woven or non-woven fabrics.

In some embodiments, the medical device is coated by or with the composition according to the present invention. In certain embodiments, the medical device according to the invention is configured to allow the controlled release of the pharmaceutical composition. In some embodiments, the medical device is in the form of a patch.

Uses and methods according to the invention may be performed in vivo or ex vivo.

In one aspect, the invention also relates to a pharmaceutical composition according to the instant invention, for use as a medicament.

The invention further relates to the use of a pharmaceutical composition according to the instant invention, for preparing or manufacturing a medicament.

A further aspect of the invention is a medicament comprising a therapeutically effective amount of at least three miRNAs selected in any one of Table 1, Table 2, Table 3, Table 4, Table 5, Table 6, Table 7, Table 8, Table 9, Table 10, Table 11 or Table 12. In one embodiment, the medicament comprises a composition according to the present invention.

In some embodiments, the pharmaceutical composition is for the prevention and/or the treatment of a tissue disorder.

In some embodiments, the tissue is selected from the group comprising bone tissue, cartilage tissue, skin tissue, muscular tissue, epithelial tissue, endothelial tissue, neural tissue, connective tissue and adipose tissue.

In some embodiments, the pharmaceutical composition is for the prevention and/or the treatment of a bone disorder and/or a cartilage disorder.

In some embodiments, the pharmaceutical composition is for the prevention and/or the treatment of a skin disorder.

Another aspect of the invention pertains to a method for the prevention and/or the treatment of a tissue disorder in an individual in need thereof, comprising the administration of an effective amount of a pharmaceutical composition according to the instant invention.

In one embodiment, the term “tissue” comprises or consists of bone, cartilage, skin, muscle, epithelium, endothelium, connective, neural and adipose tissue. Accordingly, in one embodiment, tissue disorder comprises or consists of bone, cartilage, skin, muscle, endothelium and adipose tissue disorder.

In certain embodiments, the tissue disorder is selected from the group comprising, or consisting of, aplasia cutis congenita; a burn; a cancer, including a breast cancer, a skin cancer and a bone cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer; arthritis; bone fracture; bone frailty; Caffey's disease; congenital pseudarthrosis; cranial deformation; cranial malformation; delayed union; infiltrative disorders of bone; hyperostosis; loss of bone mineral density; metabolic bone loss; osteogenesis imperfecta; osteomalacia; osteonecrosis; osteopenia; osteoporosis; Paget's disease; pseudarthrosis; sclerotic lesions; spina bifida; spondylolisthesis; spondylolysis; chondrodysplasia; costochondritis; enchondroma; hallux rigidus; hip labral tear; osteochondritis dissecans; osteochondrodysplasia; polychondritis; and the likes.

As used herein, the term “cancer” includes solid cancer. In certain embodiments, the solid cancer is selected from the group comprising, or consisting of, a bone cancer, a brain cancer, a skin cancer, a breast cancer, a cancer of the central nervous system, a cancer of the cervix, a cancer of the upper aero digestive tract, a colorectal cancer, an endometrial cancer, a germ cell cancer, a bladder cancer, a kidney cancer, a laryngeal cancer, a liver cancer, a lung cancer, a neuroblastoma, an esophageal cancer, an ovarian cancer, a pancreatic cancer, a pleural cancer, a prostate cancer, a retinoblastoma, a small intestine cancer, a soft tissue sarcoma, a stomach cancer, a testicular cancer and a thyroid cancer. In some embodiments, the tissue disorder is a soft tissue disorder. As used herein, the term “soft tissue” is intended to refer to a tissue that does not have a solid structure, and therefore is not obtained by a process of ossification and/or calcification.

In certain embodiments, the soft tissue disorder is selected from the group comprising, or consisting of aplasia cutis congenita; a bum; a cancer, including a breast cancer, a skin cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer. In one embodiment, pharmaceutical composition is for use for tissue reconstruction.

In some embodiments, the tissue reconstruction is selected from the group comprising bone reconstruction, cartilage reconstruction, skin reconstruction, muscle or myogenic reconstruction, epithelial reconstruction, endothelial reconstruction, connective reconstruction, neural reconstruction and adipogenic reconstruction.

Examples of bone and skin reconstruction include, but are not limited to, dermal and/or epidermal reconstruction, wound healing, diabetic ulcer treatment such as diabetic foot ulcer, post-burn lesions reconstruction, post-radiation lesions reconstruction, reconstruction after breast cancer or breast deformities.

Examples of cartilage reconstruction include, but are not limited to, knee chondroplasty, nose or ear reconstruction, costal or sternal reconstruction.

Examples of myogenic reconstruction include, but are not limited to, skeletal muscle reconstruction, reconstruction after break of the abdominal wall, reconstruction after ischemic muscular injury of lower limbs, reconstruction associated with compartment syndrome (CS).

Examples of endothelial reconstruction include, but are not limited to, recellularization of vascular patchs for vascular anastomosis such as venous arteriosclerosis shunt.

Examples of adipogenic reconstruction include, but are not limited to, esthetic surgery, rejuvenation, lipofilling reconstruction.

In some aspect, the invention relates to the composition or a pharmaceutical composition for use according to the invention, for skin reconstruction, preferably for treating a skin wound.

The invention also pertains to a method for skin reconstruction, preferably for treating a skin wound, in an individual in need thereof, comprising the administration of a therapeutically effective amount of a pharmaceutical composition according to the invention.

In one embodiment, the pharmaceutical composition or medical device of the invention is for use in treating skin tissue disorders. In one embodiment, the pharmaceutical composition or medical device of the invention is for use for skin reconstruction, including dermis and/or epidermis reconstruction. In one embodiment, the pharmaceutical composition or medical device of the invention is for dermal and/or epidermal reconstruction, wound healing, diabetic ulcer treatment such as diabetic foot ulcer, post-burn lesions reconstruction, post-radiation lesions reconstruction, reconstruction after breast cancer or breast deformities. In a particular embodiment, the pharmaceutical composition or medical device of the invention is for use for, or for use in treating, skin wound, preferably diabetic skin wound. In one embodiment, the pharmaceutical composition or medical device of the invention is for promoting the closure of wound. In one embodiment, the composition, pharmaceutical composition or medical device of the invention is for reducing the thickness of wound, in particular during wound healing.

In a particular embodiment, the pharmaceutical composition or medical device of the invention is for use for, or for use in treating, epidermolysis bulbosa, giant congenital nevi, and/or aplasia cutis congenita.

In still another aspect, the invention relates to the pharmaceutical composition or medical device of the invention for use for reconstructive and/or aesthetic surgery.

In one embodiment, the subject has already been treated for tissue defect. In another embodiment, the subject has not already been treated for a tissue disorder. In one embodiment, the subject was non-responsive to at least one other treatment for a tissue disorder. In one embodiment, the subject is diabetic. In one embodiment, the subject is suffering from a diabetic wound.

Another aspect of the invention also relates to a pharmaceutical composition for use according to the invention for compensating the side effects of a primary treatment of a tissue disorder. The invention further pertains to a method for compensating the side effects of a primary treatment of a tissue disorder, in an individual in need thereof, comprising the administration of a therapeutically effective amount of a pharmaceutical composition according to the invention. In certain embodiments, the primary treatment may be selected in a group comprising an anti-inflammatory treatment, a cancer treatment, the like and a combination thereof.

In another aspect, the invention also relates to a pharmaceutical composition for use according to the invention for strengthening a primary treatment of a tissue disorder. In practice, the pharmaceutical composition according to the invention may be administered prior, during or upon the primary treatment.

Another aspect of the invention also relates to a composition for use according to the invention for compensating the side effects of a therapeutic treatment known to have a deleterious effect on tissues. In certain embodiments, the said therapeutic treatment may be selected in a group comprising an anti-inflammatory treatment, a cancer treatment, an antibiotic treatment, an immunotherapy, a chemotherapy, the like and a combination thereof.

Another aspect of the invention pertains to a method for the prevention and/or the treatment of a bone disorder and/or a cartilage disorder in an individual in need thereof, comprising the administration of an effective amount of a pharmaceutical composition according to the instant invention.

In certain embodiments, said bone disorder is selected in a group of disorders comprising arthritis, bone cancer, bone fracture, bone frailty, Caffey's disease, congenital pseudarthrosis, cranial deformation, cranial malformation, delayed union, infiltrative disorders of bone, hyperostosis, loss of bone mineral density, metabolic bone loss, osteogenesis imperfecta, osteomalacia, osteonecrosis, osteopenia, osteoporosis, Paget's disease, pseudarthrosis, sclerotic lesions, spina bifida, spondylolisthesis and spondylolysis.

In some embodiments, the cartilage disorder is selected in a group comprising arthritis, chondrodysplasia, costochondritis, enchondroma, hallux rigidus, hip labral tear, osteochondritis dissecans, osteochondrodysplasia and polychondritis.

In certain embodiments, the pharmaceutical composition is for promoting osteogenesis. Another aspect of the invention pertains to a method for promoting osteogenesis in an individual in need thereof, comprising the administration of an effective amount of a pharmaceutical composition according to the instant invention.

In some embodiments, the pharmaceutical composition is for inhibiting and/or reducing osteoclastogenesis.

Another aspect of the invention pertains to a method for inhibiting and/or reducing osteoclastogenesis in an individual in need thereof, comprising the administration of an effective amount of a pharmaceutical composition according to the instant invention.

In certain embodiments, the pharmaceutical composition is for promoting chondrogenesis.

Another aspect of the invention pertains to a method for promoting chondrogenesis in an individual in need thereof, comprising the administration of an effective amount of a pharmaceutical composition according to the instant invention.

In certain embodiments, the pharmaceutical composition is for promoting angiogenesis.

In another aspect, the invention further pertains to a method for promoting angiogenesis in an individual in need thereof, comprising the administration of an effective amount of a pharmaceutical composition according to the instant invention.

In some embodiments, an individual in need thereof is an individual having or susceptible to develop a bone disorder selected in a group comprising arthritis, bone cancer, bone fracture, bone frailty, Caffey's disease, congenital pseudarthrosis, cranial deformation, cranial malformation, delayed union, infiltrative disorders of bone, hyperostosis, loss of bone mineral density, metabolic bone loss, osteogenesis imperfecta, osteomalacia, osteonecrosis, osteopenia, osteoporosis, Paget's disease, pseudarthrosis, sclerotic lesions, spina bifida, spondylolisthesis and spondylolysis.

In certain embodiments, an individual in need thereof is an individual having or susceptible to develop a cartilage disorder selected in a group comprising arthritis, chondrodysplasia, costochondritis, enchondroma, hallux rigidus, hip labral tear, osteochondritis dissecans, osteochondrodysplasia and polychondritis.

Another aspect of the invention also relates to a pharmaceutical composition for use according to the invention for compensating the side effects of a primary treatment of a bone disorder and/or a cartilage disorder.

The invention further pertains to a method for compensating the side effects of a primary treatment of a bone disorder and/or a cartilage disorder, in an individual in need thereof, comprising the administration of a therapeutically effective amount of a pharmaceutical composition according to the invention.

In certain embodiments, the primary treatment may be selected in a group comprising an anti-inflammatory treatment, a cancer treatment, in particular a solid cancer treatment, the like and a combination thereof.

In another aspect, the invention also relates to a pharmaceutical composition for use according to the invention for strengthening a primary treatment of a bone disorder and/or a cartilage disorder.

In practice, the pharmaceutical composition according to the invention may be administered prior, during or upon the primary treatment.

Another aspect of the invention also relates to a composition for use according to the invention for compensating the side effects of a therapeutic treatment known to have a deleterious effect on bones and/or cartilages.

In certain embodiments, the said therapeutic treatment may be selected in a group comprising an anti-inflammatory treatment, a cancer treatment, an antibiotic treatment, an immunotherapy, a chemotherapy, the like and a combination thereof.

In some embodiments, a pharmaceutical composition according to the invention may be formulated in any suitable form encompassed by the state in the art, e.g. in the form of an injectable solution or suspension, a tablet, a coated tablet, a capsule, a syrup, a suppository, a cream, an ointment, a lotion, a gel and the like.

In some embodiments, the pharmaceutical composition is in the form of a semi solid. In some embodiments, the pharmaceutical composition is in the form of a paste, an ointment, a cream, a plaster or a gel. In some embodiments, the pharmaceutical composition may be in the form of a moldable paste or a film that can be manipulated and grafted.

In one embodiment, the pharmaceutical composition of the invention can be processed together with suitable excipients to the semi solid form, preferably the paste. Suitable excipients are, in particular, those excipients normally used to produce paste bases. Particularly suitable according to the invention are excipients normally used to produce gel-like paste bases, such as gel formers. Gel formers are substances which form gels with a dispersant such as water. Examples of gel formers of the invention are sheet silicates, carrageenans, xanthan, gum acacia, alginates, alginic acids, pectins, modified celluloses or poloxamers.

In one embodiment, the pharmaceutical composition in a semi solid form, preferably in the form of a paste, is ready for use. In another embodiment, the pharmaceutical composition in a semi solid form, preferably in the form of a paste, has to be extemporaneously produced.

In some embodiments, the miRNAs comprised in the pharmaceutical composition of the invention are encapsulated, i.e. are immobilized in a vesicular system. In one embodiment, the encapsulation is a bilayer encapsulation. In another embodiment, the encapsulation is a single layer encapsulation. In still another embodiment, the encapsulation is a matrix encapsulation.

In one embodiment, the vesicles encapsulating the miRNAs are made of a biopolymer. In another embodiment, the vesicles encapsulating the miRNAs are extracellular vesicles. In a particular embodiment, the vesicles encapsulating the miRNAs are exosomes. Thus, in this embodiment, the pharmaceutical composition of the invention comprises miRNAs-encapsulating exosomes. In a specific embodiment, the exosomes are cells-derived exosomes, preferably exosomes from which the miRNAs are derived. In another specific embodiment, the exosomes are engineered exosomes.

Exosome engineering may be performed by any suitable methods known in the state of the art, or adapted therefrom. One may refer to, e.g., “Exosome engineering: Current progress in cargo loading and targeted delivery” (Fu et al., Nanolmplant, 2020, Volume 20, 100261).

In some embodiments, an effective amount of said active agent is administered to said individual in need thereof. Within the scope of the instant invention, an “effective amount” refers to the amount of said active agent that alone stimulates the desired outcome, i.e. alleviates or eradicates the symptoms of the tissue disorder, including the skin disorder, the bone disorder and/or cartilage disorder.

Within the scope of the instant invention, the effective amount of the active agent to be administered may be determined by a physician or an authorized person skilled in the art and can be suitably adapted within the time course of the treatment.

In certain embodiments, the effective amount to be administered may depend upon a variety of parameters, including the material selected for administration, whether the administration is in single or multiple doses, and the individual's parameters including gender, age, physical condition, size, weight, and the severity of the disorder.

In certain embodiments, an effective amount of the active agent may comprise from about 0.001 mg to about 3,000 mg, per dosage unit, preferably from about 0.05 mg to about 100 mg, per dosage unit.

Within the scope of the instant invention, from about 0.001 mg to about 3,000 mg includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg, per dosage unit.

In certain embodiments, the active agent may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day.

In certain embodiments, each dosage unit may be administered three times a day, two times a day, once a day, every other day, every three days, every week, every two weeks, every three weeks, or every four weeks.

In certain embodiments, the therapeutic treatment encompasses an administration of a plurality of dosage units, including two, three, four, five, six, seven, eight, nine, ten, eleven, twelve, thirteen, fourteen, or more administrations.

According to one embodiment, the pharmaceutical composition, medicament or medical device of the invention is administered by any suitable route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical, mucosal, nasal, buccal, sublingual; by intratracheal instillation, bronchial instillation, and/or inhalation; and/or as an oral spray, nasal spray, and/or aerosol.

In one embodiment, the pharmaceutical composition, medicament or medical device is administered at the site of the tissue disorder. In certain embodiments, the pharmaceutical composition, medicament or medical device of the invention may be administered locally, e.g., by injection, during surgery, in particular during invasive surgery.

According to one embodiment, the pharmaceutical composition, medicament or medical device of the invention is administered topically, by injection or by surgical implantation. In one embodiment, the pharmaceutical composition, medicament or medical device is administered at the site of the bone and/or cartilage disorder.

In some embodiments, the pharmaceutical compositions of the instant invention may be rehydrated before administration. Illustratively, the pharmaceutical compositions of the instant invention may be rehydrated with a sterile saline composition, in particular a sterile saline composition comprising from about 0.75% to about 1.25% NaCl, more preferably a sterile saline composition comprising from about 0.90% NaCl.

One aspect of the invention relates to a method for producing a composition comprising at least three miRNAs selected in selected in any one of Table 1, Table 2, Table 3, Table 4, Table 5, Table 6, Table 7, Table 8, Table 9, Table 10, Table 11, or Table 12, said method comprising the steps of:
1) culturing a combination comprising (i) viable cells capable to undergo differentiation, and (ii) a particulate material, so as to obtain a multidimensional structure comprising an extracellular matrix secreted by said cells, wherein the said cells have tissue regeneration and/or tissue repairing properties, wherein the cells and the particulate material are embedded in the extracellular matrix, and wherein said multidimensional structure comprises a RNA content comprising at least three miRNAs selected in selected in Table 1, Table 2, Table 3, Table 4, Table 5, Table 6, Table 7, Table 8, Table 9, Table 10, Table 11, or Table 12;
2) extracting the RNAs content produced in step 1), in particular the miRNAs content.

As used herein, the expression “viable cells capable to undergo differentiation” is intended to refer to a population of cells that can be differentiated in cells that belong to the tissue to be regenerated and/or repaired, or that possess tissue regeneration and/or tissue repair properties.

One further aspect of the invention relates to a method for producing a composition comprising at least three miRNAs selected in selected in Table 1, Table 3, Table 5, Table 7, Table 9 or Table 11, said method comprising the steps of:
1) culturing a combination comprising (i) viable cells capable to undergo differentiation, and (ii) gelatin so as to obtain a multidimensional structure comprising an extracellular matrix secreted by said cells, wherein the said cells have tissue regeneration and/or tissue repair properties, wherein the cells and the gelatin are embedded in the extracellular matrix, and wherein said multidimensional structure comprises a RNA content comprising at least three miRNAs selected in selected in Table 1, Table 3, Table 5, Table 7, Table 9 or Table 11;
2) extracting the RNAs content produced in step 1), in particular the miRNAs content.

One aspect of the invention relates to a method for producing a composition comprising at least three miRNAs selected in selected in Table 2, Table 4, Table 6, Table 8, Table 10 or Table 12, said method comprising the steps of:
1) culturing a combination comprising (i) viable cells capable to undergo osteogenic and/or chondrogenic differentiation, and (ii) a particulate material so as to obtain a multidimensional structure comprising an extracellular matrix secreted by said cells, wherein the said cells have osteogenic and/or chondrogenic properties, wherein the cells and the particulate material are embedded in the extracellular matrix, and wherein said multidimensional structure comprises a RNA content comprising at least three miRNAs selected in selected in Table 2, Table 4, Table 6, Table 8, Table 10 or Table 12;
2) extracting the RNAs content produced in step 1), in particular the miRNAs content.

As used herein, the expression “viable cells capable to undergo osteogenic and/or chondrogenic differentiation” is intended to refer to a population of cells that can be differentiated in cells that possess osteogenic and/or chondrogenic properties.

Viability of the cells according to the invention may be assessed by any suitable methods known in the state of the art, or adapted therefrom. One may refer to, e.g., “Mammalian cell viability: Methods and Protocols” (2011; Editor: M J Stoddart). Illustratively, cells may be recovered upon hydration of a desiccated composition and contacted with a suitable culturing medium in adapted culturing conditions. Viability of the cells may be assessed upon trypan blue dye exclusion staining. Alternatively, viability of the cells may be assessed upon measurement of the consumption of a carbon source, in particular glucose, in the culture medium.

As used herein, the term “embedded in” is intended to mean “enclosed closely in” or “being an integral part of”. In other words, by “cells and the particulate material are embedded in the extracellular matrix”, one may understand that the cells, the particulate material and the extracellular matrix are intimately linked one to another and that the three ingredients make one unique structure.

In certain embodiments, said cells are selected in a group comprising primary cells, stem cells, genetically modified cells, and a mixture thereof.

In practice, the cells according to the instant invention may be animal cells, preferably mammal cells, more preferably human cells.

In some embodiments, primary cells may be selected in a group comprising or consisting of osteocytes, osteoblasts, osteoclasts, chondroblasts, chondrocytes, keratinocytes, dermal fibroblasts, fibroblasts, hematopoietic cells, hepatic cells, epithelial cells, myofibroblasts, endothelial cells, connective cells, neural cells, adipocytes, and a combination thereof. In some embodiments, primary cells may be selected in a group comprising or consisting of osteocytes, osteoblasts, osteoclasts, chondroblasts, chondrocytes and a mixture thereof. Because primary cells are differentiated cells, they can be cultured in any suitable culture medium for maintenance or proliferation purposes. In some embodiments, the primary cells may be cultured in a culture medium suitable for allowing proliferation or maintenance of the cells.

In certain embodiments, stem cells may be selected in a group comprising or consisting of osteoprogenitors, embryonic stem cells (ESCs), mesenchymal stem cells (MSCs), pluripotent stem cells (pSCs) and induced pluripotent stem cells (ipSCs).

As used herein, “embryonic stem cells” (ESCs) generally refer to embryonic cells, which are capable of differentiating into cells of any one of the three embryonic germ layers, namely endoderm, ectoderm or mesoderm, or capable of being maintained in an undifferentiated state. Such cells may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation and other methods with non-fertilized eggs, such as parthenogenesis method or nuclear transfer.

In certain embodiments, the ESCs according to the invention are animal ESCs, preferably mammal ESCs, more preferably human ESCs (hESCs).

In practice, suitable ESCs may be obtained using well-known cell-culture methods. For example, ESCs can be isolated from blastocysts. Blastocysts are typically obtained from in vivo preimplantation embryos or from in vitro fertilized (IVF) embryos. Alternatively, a single cell embryo can be expanded to the blastocyst stage. Further details on methods of preparation ESCs may be found in U.S. Pat. No. 5,843,780.

In some embodiments, hESCs may advantageously be obtained without embryo destruction, as described by Chung et al. (2008)). In some embodiments, hESCs may be advantageously obtained from embryo collected or isolated less than 14 days upon fertilization. In some embodiments, the ESCs are not human ESCs.

As used herein, “mesenchymal stem cells” (MSCs) generally refer to stromal cells from a specialized tissue (also named differentiated tissue) and capable of self-renewal (i.e. making identical copies of themselves) for the lifetime of the organism and have multipotent differentiation potential.

In some embodiments, the MSCs according to the invention are animal MSCs, preferably mammal MSCs, more preferably human MSCs (hMSCs). In practice, hMSCs suitable for implementing the instant invention thus encompass any suitable human multipotent stem cells derived from any suitable tissue, using any appropriate isolation method. Illustratively, hMSCs encompass, but are not limited to, adult multilineage inducible (MIAMI) cells (D'Ippolito et al.; 2004), cord blood derived stem cells (Kogler et al.; 2004), mesoangioblasts (Sampaolesi et al.; 2006; Dellavalle et al.; 2007), and amniotic stem cells (De Coppi et al.; 2007). Furthermore, umbilical cord blood banks (e.g., Etablissement Francais du Sang, France) provide secure and easily available sources of such cells for transplantation. In certain embodiments, the MSCs according to the invention are pre-osteoblasts or pre-chondroblasts.

In some embodiments, the mesenchymal stem cells are adipose tissue-derived stem cells (ASCs). As used herein, the following terms are considered to refer to ASCs: Adipose tissue-derived Stem/Stromal Cells (ASCs); Adipose Derived Adult Stem (ADAS) Cells, Adipose Derived Adult Stromal Cells, Adipose Derived Stromal Cells (ADSC), Adipose Stromal Cells (ASC), Adipose Mesenchymal Stem Cells (AdMSC), Lipoblasts, Pericytes, Pre-Adipocytes, Processed Lipoaspirate (PLA) Cells.

In one embodiment, ASCs tissue is of animal origin, preferably of mammal origin, more preferably of human origin. Accordingly, in one embodiment, ASCs are animal ASCs, preferably mammal ASCs, more preferably human ASCs. In a preferred embodiment, ASCs are human ASCs.

Methods of isolating stem cells from adipose tissue are known in the art, and are disclosed for example in Zuk et al. (Tissue Engineering. 2001, 7:211-228). In one embodiment, ASCs are isolated from adipose tissue by liposuction.

As an illustration, adipose tissue may be collected by needle biopsy or liposuction aspiration. ASCs may be isolated from adipose tissue by first washing the tissue sample extensively with phosphate-buffered saline (PBS), optionally containing antibiotics, for example 1% Penicillin/Streptomycin (P/S). Then the sample may be placed in a sterile tissue culture plate, or a sterile tube, with collagenase for tissue digestion (for example, Collagenase Type I prepared in PBS containing 2% P/S), and incubated for 60 min at 37° C., 5% CO₂, in a water bath, with manual shaking every 20 min. The collagenase activity may be neutralized by adding culture medium (for example DMEM containing 10% human platelet lysate (hPL)). Upon disintegration, the sample may be transferred to a tube. The stromal vascular fraction (SVF), containing the ASCs, is obtained by centrifuging the sample (for example at 2,000 rpm for 5 min). To complete the separation of the stromal cells from the primary adipocytes, the sample may be shaken vigorously to thoroughly disrupt the pellet and to mix the cells. The centrifugation step may be repeated. After spinning and the collagenase solution aspirate, the pellet may be resuspended in lysis buffer, incubated on ice (for example for 10 min), washed (for example with PBS/2% P/S) and centrifuged (for example at 2,000 rpm for 5 min). The supernatant may be then aspirated, the cell pellet resuspended in medium (for example, stromal medium, i.e., α-MEM, supplemented with 20% FBS, 1% L-glutamine, and 1% P/S), and the cell suspension filtered (for example, through 70 um cell strainer). The sample containing the cells may be finally plated in culture plates and incubated at 37° C., 5% CO₂.

In one embodiment, ASCs of the invention are isolated from the stromal vascular fraction of adipose tissue. In one embodiment, the lipoaspirate may be kept several hours at room temperature, or at +4° C. for 24-72 hours prior to use, or below 0° C., for example −18° C. or −80° C., for long-term conservation.

In one embodiment, ASCs may be fresh ASCs or refrigerated ASCs. Fresh ASCs are isolated ASCs which have not undergone a refrigerating treatment. Refrigerated ASCs are isolated ASCs which have undergone a refrigerating treatment. In one embodiment, a refrigerating treatment means any treatment below 0° C. In one embodiment, the refrigerating treatment may be performed at about −18° C., at −80° C. or at −180° C. In a specific embodiment, the refrigerating treatment may be cryopreservation.

As an illustration of refrigerating treatment, ASCs may be harvested at 80-90% confluence. After steps of washing and detachment from the dish, cells may be pelleted at 20° C. with a refrigerating preservation medium and placed in vials. In one embodiment, the refrigerating preservation medium comprises 80% fetal bovine serum or human serum, 10% dimethylsulfoxide (DMSO) and 10% DMEM/Ham's F-12. Then, vials may be stored at −80° C. overnight. For example, vials may be placed in an alcohol freezing container which cools the vials slowly, at approximately 1° C. every minute, until reaching −80° C. Finally, frozen vials may be transferred to a liquid nitrogen container for long-term storage.

In one embodiment, ASCs are differentiated ASCs. In a preferred embodiment, ASCs are osteogenic differentiated ACSs. In other words, in a preferred embodiment, ASCs are differentiated into osteogenic cells. In a particular embodiment, ASCs are differentiated into osteoblasts and/or osteocytes. As used herein, the term “differentiated” when referred to stem cells, in particular ASCs, is intended to mean that the cells are in a mature form and possess the characteristics of cells physiologically found in a given tissue. The differentiated cells underwent a differentiation process, and the population of differentiated cells may be partially or fully differentiated.

In another embodiment, ASCs are chondrogenic differentiated ACSs. In other words, in one embodiment, ASCs are differentiated into chondrogenic cells. In a particular embodiment, ASCs are differentiated into chondrocytes.

As used herein, the term “pluripotent” refers to cells having the capacity to generate a cellular progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8 to 12 weeks-old SCID mice, can be used to establish the pluripotency of a cell population. However, identification of various pluripotent stem cell characteristics can also be used to identify pluripotent cells. In some embodiments of the invention, the pluripotent stem cells are animal pluripotent stem cells, preferably mammal pluripotent stem cells, more preferably human pluripotent stem cells.

As used herein, an “induced pluripotent stem cell” (iPSC) refers to a pluripotent stem cell artificially derived from a non-pluripotent cell. A non-pluripotent cell may be a cell of lesser ability (or potency) to self-renew and to differentiate as compared to a pluripotent stem cell. Cells of lesser potency may be, but are not limited to, somatic stem cells, tissue specific progenitor cells, primary or secondary cells. In some embodiments, the iPSCs are human iPSCs (hiPSCs).

In some embodiments, the cells comprise genetically modified cells. In practice genetically modified cells are engineered so as to synthesize the factors and the nucleic acids that promote tissue regeneration and/or tissue repair properties.

Within the scope of the instant invention, the expression “genetically modified” is intended to refer to a cell that possesses one or more nucleotide substitution, addition or deletion in its genome and/or comprises one or more additional extra chromosomic nucleic acids encoding one or more factors interfering with the physiological outcome of the cell's fate. In certain embodiments, the genetically modified cells are of animal origin, preferably of mammal origin, more preferably of human origin.

On the contrary, because stem cells and genetically modified cells are not differentiated cells, they may undergo a differentiation, including, but not limited to an osteogenic and/or chondrogenic differentiation process.

In some embodiments, the differentiation comprises osteogenic differentiation, chondrogenic differentiation, keratinogenic differentiation, epithelial differentiation, endothelial differentiation, myofibrogenic differentiation, connective tissue differentiation, neural differentiation, adipogenic differentiation, the like and a combination thereof.

In one embodiment, cells, in particular ASCs, are differentiated. In a preferred embodiment, cells, in particular ASCs, are osteogenic differentiated. In other words, in a preferred embodiment, cells, in particular ASCs, are differentiated into osteogenic cells. In a particular embodiment, cells, in particular ASCs, are differentiated into osteoblasts and/or osteocytes, or precursor cells thereof.

Methods to control and assess the osteogenic differentiation are known in the art. For example, the osteo-differentiation of the cells or tissues of the invention may be assessed by staining of osteocalcin and/or phosphate (e.g., with von Kossa); by staining calcium phosphate (e.g., with Alizarin red); by magnetic resonance imaging (MRI); by measurement of mineralized matrix formation; or by measurement of alkaline phosphatase activity.

In one embodiment, osteogenic differentiation of stem cells or genetically modified cells, in particular ASCs, is performed by culture of cells in osteogenic differentiation medium (MD). In one embodiment, the osteogenic differentiation medium comprises human serum. In a particular embodiment, the osteogenic differentiation medium comprises human platelet lysate (hPL). In one embodiment, the osteogenic differentiation medium does not comprise any other animal serum, preferably it comprises no other serum than human serum.

In one embodiment, the osteogenic differentiation medium comprises or consists of proliferation medium supplemented with dexamethasone, ascorbic acid and sodium phosphate. In one embodiment, the osteogenic differentiation medium further comprises antibiotics, such as penicillin, streptomycin, gentamycin and/or amphotericin B. In one embodiment, all media are free of animal proteins.

In one embodiment, proliferation medium may be any culture medium designed to support the growth of the cells known to one of ordinary skill in the art. As used herein, the proliferation medium is also called “growth medium”. Examples of growth medium include, without limitation, RPMI, MEM, DMEM, IMDM, RPMI 1640, FGM or FGM-2, 199/109 medium, HamF10/HamF12 or McCoy's 5A. In a preferred embodiment, the proliferation medium is DMEM.

In one embodiment, the osteogenic differentiation medium comprises or consists of DMEM supplemented with L-alanyl-L-glutamine (Ala-Gln, also called ‘Glutamax®’ or ‘Ultraglutamine®’), hPL, dexamethasone, ascorbic acid and sodium phosphate. In one embodiment, the osteogenic differentiation medium comprises or consists of DMEM supplemented with L-alanyl-L-glutamine, hPL, dexamethasone, ascorbic and sodium phosphate, and antibiotics, preferably penicillin, streptomycin, gentamycin and/or amphotericin B.

In one embodiment, the osteogenic differentiation medium comprises or consists of DMEM supplemented with L-alanyl-L-glutamine, hPL (about 5%, v/v), dexamethasone (about 1 μM), ascorbic acid (about 0.25 mM) and sodium phosphate (about 2.93 mM). In one embodiment, the osteogenic differentiation medium comprises or consists of DMEM supplemented with L-alanyl-L-glutamine, hPL (about 5%, v/v), dexamethasone (about 1 μM), ascorbic acid (about 0.25 mM) and sodium phosphate (about 2.93 mM), penicillin (about 100 U/mL) and streptomycin (about 100 μg/mL). In one embodiment, the osteogenic differentiation medium further comprises amphotericin B (about 0.1%). In one embodiment, the osteogenic differentiation medium consists of DMEM supplemented with L-alanyl-L-glutamine, hPL (about 5%, v/v), dexamethasone (about 1 μM), ascorbic acid (about 0.25 mM) and sodium phosphate (about 2.93 mM). In one embodiment, the osteogenic differentiation medium comprises or consists of DMEM supplemented with L-alanyl-L-glutamine, hPL (about 5%, v/v), dexamethasone (about 1 μM), ascorbic acid (about 0.25 mM) and sodium phosphate (about 2.93 mM), penicillin (about 100 U/mL), streptomycin (about 100 μg/mL) and amphotericin B (about 0.1%).

In another embodiment, the cells, in particular ASCs, are chondrogenic differentiated. In other words, in one embodiment, cells, in particular ASCs, are differentiated into chondrogenic cells. In a particular embodiment, cells, in particular ASCs, are differentiated into chondrocytes, or precursor cells thereof.

Methods to control and assess the chondrogenic differentiation are known in the art. For example, the chondro-differentiation of the cells or tissues of the invention may be assessed by measurement of the expression level of chondrocyte-specific genes such as aggrecan, collagen II and SOX-9. Methods include, but are not limited to, real-time PCR or histological analysis (e.g., staining of Alcian Blue).

In one embodiment, the chondrogenic differentiation medium comprises or consists of proliferation medium supplemented with sodium pyruvate, ascorbic acid and dexamethasone. In one embodiment, the chondrogenic differentiation medium further comprises antibiotics, such as penicillin, streptomycin, gentamycin and/or amphotericin B. In one embodiment, the chondrogenic differentiation medium further comprises growth factors, such as IGF and TGF-β. In one embodiment, all media are free of animal proteins.

In one embodiment, the chondrogenic differentiation medium comprises or consists of DMEM supplemented with hPL, dexamethasone, ascorbic acid and sodium pyruvate. In one embodiment, the chondrogenic differentiation medium may further comprise proline and/or growth factors and/or antibiotics.

In one embodiment, chondrogenic differentiation is performed by culture of ASCs in chondrogenic differentiation medium.

In one embodiment, the chondrogenic differentiation medium comprises or consists of DMEM, hPL, sodium pyruvate, ITS, proline, TGF-β1 and dexamethazone. In one embodiment, the chondrogenic differentiation medium further comprises antibiotics, such as penicillin, streptomycin, gentamycin and/or amphotericin B.

In one embodiment, the chondrogenic differentiation medium comprises or consists of DMEM, hPL (about 5%, v/v), dexamethasone (about 1 μM), sodium pyruvate (about 100 μg/mL), ITS (about 1×), proline (about 40 μg/mL) and TGF-β1 (about 10 ng/mL).

In another embodiment, cells, in particular ASCs are keratinogenic differentiated. In other words, in a preferred embodiment, cells, in particular ASCs are differentiated into keratinogenic cells. In still other words, in a preferred embodiment, cells, in particular ASCs are differentiated in keratinogenic medium. In a particular embodiment, cells, in particular ASCs are differentiated into keratinocytes, or precursor cells thereof.

Methods to control and assess the keratinogenic differentiation are known in the art. For example, the keratinogenic differentiation of the cells or tissues of the invention may be assessed by staining of Pankeratin or CD34.

In one embodiment, differentiation into keratinocytes are performed by culture of ASCs in keratinogenic differentiation medium.

In one embodiment, the keratinogenic differentiation medium comprises or consists of DMEM, hPL, insulin, KGF, hEGF, hydrocortisone and CaCl₂. In one embodiment, the keratinogenic differentiation medium further comprises antibiotics, such as penicillin, streptomycin, gentamycin and/or amphotericin B.

In one embodiment, the keratinogenic differentiation medium comprises or consists of DMEM, hPL (about 5%, v/v), insulin (about 5 μg/mL), KGF (about 10 ng/mL), hEGF (about 10 ng/mL), hydrocortisone (about 0.5 μg/mL) and CaCl₂ (about 1.5 mM).

In another embodiment, cells, in particular ASCs, are endothelial differentiated.

In still other words, in a preferred embodiment, cells, in particular ASCs, are differentiated in endothelial medium. In a particular embodiment, cells, in particular ASCs, are differentiated into endothelial cells, or precursor cells thereof.

Methods to control and assess the endothelial differentiation are known in the art. For example, the endothelial differentiation of the cells or tissues of the invention may be assessed by staining of CD34.

In one embodiment, differentiation into endothelial cells are performed by culture of cells, in particular ASCs, in endothelial differentiation medium.

In one embodiment, the endothelial differentiation medium comprises or consists of EBMTM-2 medium, hPL, hEGF, VEGF, R3-IGF-1, ascorbic acid, hydrocortisone and hFGFb. In one embodiment, the endothelial differentiation medium further comprises antibiotics, such as penicillin, streptomycin, gentamycin and/or amphotericin B.

In one embodiment, the endothelial differentiation medium comprises or consists of EBMTM-2 medium, hPL (about 5%, v/v), hEGF (about 0.5 mL), VEGF (about 0.5 mL), R3-IGF-1 (about 0.5 mL), ascorbic acid (about 0.5 mL), hydrocortisone (about 0.2 mL) and hFGFb (about 2 mL), reagents of the kit Clonetics™ EGM™-2MV BulletKit™ CC-3202 (Lonza®).

In another embodiment, cells, in particular ASCs, are myofibrogenic differentiated. In other words, in a preferred embodiment, the cells, in particular ASCs, are differentiated into myofibrogenic cells. In still other words, in a preferred embodiment, the cells, in particular ASCs, are differentiated in myofibrogenic medium. In a particular embodiment, the cells, in particular ASCs, are differentiated into myofibroblasts, or precursor cells thereof.

Methods to control and assess the myofibrogenic differentiation are known in the art. For example, the myofibrogenic differentiation of the cells or tissues of the invention may be assessed by staining of α-SMA.

In one embodiment, differentiation into myofibrogenic cells are performed by culture of cells, in particular ASCs, in myofibrogenic differentiation medium.

In one embodiment, the myofibrogenic differentiation medium comprises or consists of DMEM:F12, sodium pyruvate, ITS, RPMI 1640 vitamin, TGF-β1, Glutathione, MEM. In one embodiment, the myofibrogenic differentiation medium further comprises antibiotics, such as penicillin, streptomycin, gentamycin and/or amphotericin B.

In one embodiment, the myofibrogenic differentiation medium comprises or consists of DMEM:F12, sodium pyruvate (about 100 μg/mL), ITS (about 1×), RPMI 1640 vitamin (about 1×), TGF-β1 (about 1 ng/mL), Glutathione (about 1 μg/mL), MEM (about 0.1 mM).

In another embodiment, cells, in particular ASCs, are adipogenic differentiated. In other words, in a preferred embodiment, the cells, in particular ASCs, are differentiated into adipogenic cells. In still other words, in a preferred embodiment, the cells, in particular ASCs, are differentiated in adipogenic medium. In a particular embodiment, the cells, in particular ASCs, are differentiated into adipocytes, or precursor cells thereof.

Methods to control and assess the adipogenic differentiation are known in the art. For example, the adipogenic differentiation of the cells or tissues of the invention may be assessed by staining by Oil-Red.

In one embodiment, differentiation into adipocytes are performed by culture of ASCs in adipogenic differentiation medium.

In one embodiment, the adipogenic differentiation medium comprises or consists of DMEM, hPL, Dexamethazone, insulin, Indomethacin and IBMX. In one embodiment, the adipogenic differentiation medium further comprises antibiotics, such as penicillin, streptomycin, gentamycin and/or amphotericin B.

In one embodiment, the adipogenic differentiation medium comprises or consists of DMEM, hPL (about 5%), Dexamethazone (about 1 μM), insulin (about 5 μg/mL), Indomethacin (about 50 μM) and IBMX (about 0.5 mM).

In another embodiment, cells, in particular ASCs, are neural differentiated. In other words, in a preferred embodiment, the cells, in particular ASCs, are differentiated into neural cells. In a particular embodiment, the cells, in particular ASCs, are differentiated into neural cells. In a specific embodiment, the cells, in particular ASCs, are differentiated into neurons. In another specific embodiment, the cells, in particular ASCs, are differentiated into glial cells.

In one embodiment, differentiation into neural cells are performed by culture of the cells, in particular ASCs, in neurons or glial cells differentiation medium.

Methods to control and assess the neural differentiation are known in the art. For example, the neural differentiation of the cells or tissues of the invention may be assessed according to the morphology, physiology, or global gene expression pattern. For instance, the neural differentiation of the cells or tissues of the invention may be assessed by the cell growth in length, by the development of a growth cone, and/or by staining of neuroectodermal stem cell markers including NESTIN, PAX6, and SOX2. Another method to control and assess the neural differentiation is to assess the electrophysiological profile of the differentiated cells.

In one embodiment, the cells, in particular ASCs, are late passaged adipose tissue-derived stem cells. As used herein, the term “late passages” means adipose tissue-derived stem cells differentiated at least after passage 4. As used herein, the passage 4 refers to the fourth passage, i.e., the fourth act of splitting cells by detaching them from the surface of the culture vessel before they are resuspended in fresh medium. In one embodiment, late passaged adipose tissue-derived stem cells are differentiated after passage 4, passage 5, passage 6 or more. In a preferred embodiment, cells, in particular ASCs, are differentiated after passage 4.

As used herein, the term “vessel” means any cell culture surface, such as for example a flask or a well-plate.

The initial passage of the primary cells was referred to as passage 0 (P0). According to the present invention, passage PO refers to the seeding of cell suspension from the pelleted Stromal Vascular Fraction (SVF) on culture vessels. Therefore, passage P4 means that cells were detached 4 times (at P1, P2, P3 and P4) from the surface of the culture vessel (for example by digestion with trypsin) and resuspended in fresh medium.

In one embodiment, the cells of the invention, in particular ASCs, are cultured in proliferation medium up to the fourth passage. In one embodiment, the cells of the invention, in particular ASCs, are cultured in differentiation medium after the fourth passage. Accordingly, in one embodiment, at passages P1, P2 and P3, the cells of the invention, in particular ASCs are detached from the surface of the culture vessel and then diluted to the appropriate cell density in proliferation medium. Still according to this embodiment, at passage P4, cells, in particular ASCs, are detached from the surface of the culture vessel and then diluted to the appropriate cell density in differentiation medium. Therefore, according to this embodiment, at P4 the cells of the invention, in particular ASCs, are not resuspended and cultured in proliferation medium until they reach confluence before being differentiated (i.e., before being cultured in differentiation medium), but are directly resuspended and cultured in differentiation medium.

In one embodiment, cells are maintained in differentiation medium at least until they reach confluence, preferably between 70% and 100% confluence, more preferably between 80% and 95% confluence. In one embodiment, cells are maintained in differentiation medium for at least 5 days, preferably at least 10 days, more preferably at least 15 days. In one embodiment, cells are maintained in differentiation medium from 5 days to 30 days, preferably from 10 days to 25 days, more preferably from 15 days to 20 days. In one embodiment, differentiation medium is replaced every 2 days. However, as it is known in the art, the cell growth rate from one donor to another could slightly differ. Thus, the duration of the differentiation and the number of medium changes may vary from one donor to another.

In one embodiment, cells are maintained in osteogenic differentiation medium at least until formation of osteoid, i.e., the unmineralized, organic portion of the bone matrix that forms prior to the maturation of bone tissue. In one embodiment, cells are maintained in chondrogenic differentiation medium at least until formation of cartilage, immature or mature, with viscoelastic properties.

In some embodiments, the combination comprises genetically modified cells. In practice genetically modified cells are engineered so as to synthesize the factors and the nucleic acids that promote osteogenic and/or chondrogenic properties.

In some embodiments, the genetically modified cells are engineered so as to allow the synthesis of one or more growth factor, transcription factor or RNAs involved in osteogenesis and/or chondrogenesis.

In certain embodiments, the combination of step 1) comprises from about 10² to about 10¹⁶ cells per gram of the combination, preferably from about 10⁶ to about 10¹² cells per gram of the combination. Within the scope of the instant invention, the expression “from about 10² to about 10¹⁶ cells” encompasses 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 101², 5×10¹², 10¹³, 5×10¹³, 10¹⁴, 5×10¹⁴, 10¹⁵, 5×10¹⁵ and 10¹⁶ cells.

As used herein, a “culture medium” refers to the generally accepted definition in the field of cellular biology, i.e., any medium suitable for promoting the growth of the cells of interest.

In some embodiments, a suitable culture medium may include a chemically defined medium, i.e., a nutritive medium only containing specified components, preferably components of known chemical structure.

In some embodiments, a chemically defined medium may be a serum-free and/or feeder-free medium. As used herein, a “serum-free” medium refers to a culture medium containing no added serum. As used herein, a “feeder-free” medium refers to a culture medium containing no added feeder cells.

A culture medium for use according to the invention may be an aqueous medium that may include a combination of substances such as one or more salts, carbon sources, amino acids, vitamins, minerals, reducing agents, buffering agents, lipids, nucleosides, antibiotics, cytokines, and growth factors.

Examples of suitable culture media include, without being limited to, RPMI medium, William's E medium, Basal Medium Eagle (BME), Eagle's Minimum Essential Medium (EMEM), Minimum Essential Medium (MEM), Dulbecco's Modified Eagles Medium (DMEM), Ham's F-10, Ham's F-12 medium, Kaighn's modified Ham's F-12 medium, DMEM/F-12 medium, and McCoy's 5A medium, which may be further supplemented with any one of the above mentioned substances.

In some embodiments, a culture medium according to the invention may be a synthetic culture medium such as the RPMI (Roswell Park Memorial Institute medium) or the CMRL-1066 (Connaught Medical Research Laboratory).

In practice, both media may be supplemented with additional additives, commonly used in the field. In some embodiments, the additional additives may be intended to promote osteogenesis and/or chondrogenesis. Non-limitative examples of suitable additional additives encompass growth factors, transcription factors, osteocytes activators, osteoblasts activators, osteoclasts inhibitors, chondrocytes activators, the likes and a mixture thereof.

In practice, the culture parameters such as the temperature, the pH, the salinity, and the levels of O₂ and CO₂ are adjusted accordingly to the standards established in the state of the art. Illustratively, the temperature for culturing the cells according to the invention may range from about 30° C. to about 42° C., preferably from about 35° C. to about 40° C., and more preferably from about 36° C. to about 38° C. Within the scope of the invention, the expression “from about 30° C. to about 42° C.” encompasses 30° C., 31° C., 32° C., 33° C., 34° C., 35° C., 36° C., 37° C., 38° C., 39° C., 40° C., 41° C. and 42° C.

In some embodiments, the level of CO₂ during the course of culture is maintained constant and ranges from about 1% to about 10%, preferably from about 2.5% to about 7.5%. Within the scope of the invention, the expression “from about 1% to about 10%” encompasses 1, 2, 3, 4, 5, 6, 7, 8, 9 and 10%.

In one embodiment, the particulate material of the invention is in form of particles. In one embodiment, particles may be beads, powder, spheres, microspheres, and the like.

In some embodiments, the particulate material of the invention is formed by a material that provides a structural support for the growth and propagation of cells. In one embodiment, particulate material is biocompatible, and comprises a natural or synthetic material, or a chemical-derivative thereof.

Within the scope of the instant invention, “biocompatible” refers to the quality of not having toxic or injurious effects on the body.

In one embodiment, the particulate material of the invention is not structured to form a predefined 3D shape or scaffold, such as for example a cube. In one embodiment, the particulate material of the invention has not a predefined shape or scaffold. In one embodiment, the particulate material of the invention has not the form of a cube. In one embodiment, the particulate material is not a 3D scaffold. In one embodiment, the particulate material of the invention is scaffold-free.

In certain embodiments, the particulate material is selected from the group comprising or consisting of:
an organic material, including demineralized bone matrix (DBM), gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins, cellulose derivatives, or combinations thereof;
a ceramic material, including particles of calcium phosphate (CaP), calcium carbonate (CaCO₃), calcium sulfate (CaSO₄), or calcium hydroxide (Ca(OH)₂), or combinations thereof;
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example poly(propylene fumarate) (PPF) or poly(propylene fumarate-co-ethylene glycol) (P(PF-co-EG)), oligo(poly(ethylene glycol) fumarate) (OPF), poly (n-isopropylacrylamide) (PNIPPAAm), poly(aldehyde guluronate) (PAG), poly(n-vinyl pyrrolidone) (PNVP), or combination thereof
a gel, including a self-assembling oligopeptide gel, a hydrogel material, a microgel, a nanogel, a particulate gel, a hydrogel material, a thixotropic gel, a xerogel, a responsive gel, or combinations thereof;
a creamer;
and any combination thereof.

In some embodiments, the particulate material is gelatin or a ceramic material.

In one embodiment, the particulate material of the invention is gelatin.

In one embodiment, the gelatin of the invention is animal gelatin, preferably mammal gelatin, more preferably porcine gelatin. As used herein, the term “porcine gelatin” may be replaced by “pork gelatin” or “pig gelatin”. In one embodiment, the gelatin is porcine skin gelatin.

In certain embodiments, said gelatin is in the form of particles, preferably particles having a mean diameter ranging from about 50 μm to about 1,000 μm. Within the scope of the invention, the expression “from about 50 μm to about 1,000 μm” encompasses 50 μm, 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm and 1,000 μm.

In one embodiment, the gelatin of the invention is in form of particles, beads, spheres, microspheres, and the like.

In one embodiment, the gelatin of the invention is not structured to form a predefined 3D shape or scaffold, such as for example a cube. In one embodiment, the gelatin of the invention has not a predefined shape or scaffold. In one embodiment, the gelatin of the invention has not the form of a cube. In one embodiment, the gelatin, preferably the porcine gelatin, is not a 3D scaffold.

In one embodiment, the gelatin of the invention is a macroporous microcarrier.

Examples of porcine gelatin particles include, but are not limited to, Cultispher® G, Cultispher® S, Spongostan and Cutanplast. In one embodiment, the gelatin of the invention is Cultispher® G or Cultispher® S.

In one embodiment, the gelatin, preferably the porcine gelatin, of the invention have a mean diameter of at least about 50 μm, preferably of at least about 75 μm, more preferably of at least about 100 μm, more preferably of at least about 130 μm. In one embodiment, the gelatin of the invention, preferably the porcine gelatin, have a mean diameter of at most about 1,000 μm, preferably of at most about 750 μm, more preferably of at most about 500 μm. In another embodiment, the gelatin of the invention, preferably the porcine gelatin, have a mean diameter of at most about 450 μm, preferably of at most about 400 μm, more preferably of at least most about 380 μm.

In one embodiment, the gelatin of the invention, preferably the porcine gelatin, has a mean diameter ranging from about 50 μm to about 1,000 μm, preferably from about 75 μm to about 750 μm, more preferably from about 100 μm to about 500 μm. In another embodiment, the gelatin of the invention, preferably the porcine gelatin, has a mean diameter ranging from about 50 μm to about 500 μm, preferably from about 75 μm to about 450 μm, more preferably from about 100 μm to about 400 μm. In another embodiment, the gelatin of the invention, preferably the porcine gelatin, have a mean diameter ranging from about 130 μm to about 380 μm.

Methods to assess the mean diameter of gelatin particles according to the invention are known in the art. Examples of such methods include, but are not limited to, granulometry, in particular using suitable sieves; sedimentometry; centrifugation techniques; laser diffraction; and images analysis, in particular by the means of a high-performance camera with telecentric lenses; and the like.

In one embodiment, gelatin is added at a concentration ranging from about 0.1 cm³ to about 5 cm³ for a 150 cm² vessel, preferably from about 0.5 cm³ to about 4 cm³, more preferably from about 0.75 cm³ to about 3 cm³. In one embodiment, gelatin is added at a concentration ranging from about 1 cm³ to about 2 cm³ for a 150 cm² vessel. In one embodiment, gelatin is added at a concentration of about 1 cm³, 1.5 cm³ or 2 cm³ for a 150 cm² vessel. Within the scope of the invention, the expression “0.1 cm³ to about 5 cm³” encompasses 0.1 cm³, 0.2 cm³, 0.3 cm³, 0.4 cm³, 0.5 cm³, 0.6 cm³, 0.7 cm³, 0.8 cm³, 0.9 cm³, 1.0 cm³, 1.5 cm³, 2.0 cm³, 2.5 cm³, 3.0 cm³, 3.5 cm³, 4.0 cm³, 4.5 cm³ and 5.0 cm³.

In one embodiment, gelatin is added at a concentration ranging from about 0.1 g to about 5 g for a 150 cm² vessel, preferably from about 0.5 g to about 4 g, more preferably from about 0.75 g to about 3 g. In one embodiment, gelatin is added at a concentration ranging from about 1 g to about 2 g for a 150 cm² vessel. In one embodiment, gelatin is added at a concentration of about 1 g, 1.5 g or 2 g for a 150 cm² vessel. Within the scope of the invention, the expression “0.1 g to about 5 g” encompasses 0.1 g, 0.2 g, 0.3 g, 0.4 g, 0.5 g, 0.6 g, 0.7 g, 0.8 g, 0.9 g, 1.0 g, 1.5 g, 2.0 g, 2.5 g, 3.0 g, 3.5 g, 4.0 g, 4.5 g and 5.0 g.

In one embodiment, the gelatin of the invention is added to the culture medium after differentiation of the cells. In one embodiment, the gelatin of the invention is added to the culture medium when cells are sub-confluent. In one embodiment, the gelatin of the invention is added to the culture medium when cells are overconfluent. In one embodiment, the gelatin of the invention is added to the culture medium when cells have reached confluence after differentiation. In other words, in one embodiment, the gelatin of the invention is added to the culture medium when cells have reached confluence in differentiation medium. In one embodiment, the gelatin of the invention is added to the culture medium at least 5 days after P4, preferably 10 days after P4, more preferably 15 days after P4. In one embodiment, the gelatin of the invention is added to the culture medium from 5 to 30 days after P4, preferably from 10 to 25 days after P4, more preferably from 15 to 20 days after P4.

In another embodiment, the particulate material of the invention is a ceramic material. In one embodiment, the ceramic material of the invention are particles of calcium phosphate (CaP), calcium carbonate (CaCO₃), calcium sulfate (CaSO₄), or calcium hydroxide (Ca(OH)₂), or combinations thereof.

Examples of calcium phosphate particles include, but are not limited to, hydroxyapatite (HA, Ca₁₀(PO₄)₆(OH)₂), tricalcium phosphate (TCP, Ca₃(PO₄)₂), α-tricalcium phosphate (α-TCP, (α-Ca₃(PO₄)₂), β-tricalcium phosphate (β-TCP, β-Ca₃(PO₄)₂), tetracalcium phosphate (TTCP, Ca₄(PO₄)₂O), octacalcium phosphate (Ca₈H₂(PO₄)₆.5H₂O), amorphous calcium phosphate (Ca₃(PO₄)₂), hydroxyapatite/β-tricalcium phosphate (HA/β-TCP), hydroxyapatite/tetracalcium phosphate (HA/TTCP), and the like.

In one embodiment, the ceramic material of the invention comprises or consists of hydroxyapatite (HA), tricalcium phosphate (TCP), hydroxyapatite/β-tricalcium phosphate (HA/β-TCP), calcium sulfate (CaSO₄), or combinations thereof. In one embodiment, the ceramic material of the invention comprises or consists of hydroxyapatite (HA), β-tricalcium phosphate (β-TCP), hydroxyapatite/β-tricalcium phosphate (HA/β-TCP), α-tricalcium phosphate (α-TCP), calcium sulfate (CaSO₄), or combinations thereof.

In some embodiments, said particulate material comprises a ceramic material, preferably comprising calcium phosphate, preferably hydroxyapatite (HA) and/or β-tricalcium phosphate (β-TCP), more preferably particles of calcium phosphate.

In certain embodiments, said ceramic material comprises calcium phosphate, preferably hydroxyapatite (HA) and/or β-tricalcium phosphate (β-TCP), more preferably particles of calcium phosphate.

In one embodiment, the ceramic particles of the invention are particles of hydroxyapatite (HA). In another embodiment, the ceramic particles of the invention are particles of β-tricalcium phosphate (β-TCP). In another embodiment, the ceramic particles of the invention are particles of hydroxyapatite/β-tricalcium phosphate (HA/β-TCP). In other words, in one embodiment, the ceramic particles of the invention are a mixture of hydroxyapatite and β-tricalcium phosphate particles (called HA/β-TCP particles). In one embodiment, the ceramic particles of the invention consist of hydroxyapatite particles and β-tricalcium phosphate particles (called HA/β-TCP particles).

In one embodiment, the particulate material, preferably the ceramic particles, more preferably HA, β-TCP and/or HA/β-TCP particles, are in form of granules, powder or beads. In one embodiment, the particulate material, preferably the ceramic particles, more preferably HA, β-TCP and/or HA/β-TCP particles, are in form of porous granules, powder or beads. In one embodiment, the particulate material, preferably the ceramic particles, more preferably HA, β-TCP and/or HA/β-TCP particles, are porous ceramic material. In one embodiment, the particulate material, preferably the ceramic particles, more preferably HA, β-TCP and/or HA/β-TCP particles, are powder particles. In a particular embodiment, the particulate material, preferably the ceramic particles, more preferably HA, β-TCP and/or HA/β-TCP particles, are in form of porous granules. In another particular embodiment, the particulate material, preferably the ceramic particles, more preferably HA, β-TCP and/or HA/β-TCP particles, are in form of powder. In one embodiment, the particulate material, preferably the ceramic particles, more preferably HA, β-TCP and/or HA/β-TCP particles, are not structured to form a predefined 3D shape or scaffold, such as for example a cube. In one embodiment, the particulate material, preferably the ceramic material of the invention is not a 3D scaffold. In one embodiment, the particulate material, preferably the ceramic material has not a predefined shape or scaffold. In one embodiment, the particulate material, preferably the ceramic material of the invention has not the form of a cube.

In one embodiment, the particulate material, preferably the ceramic particles of the invention, more preferably HA, β-TCP and/or HA/β-TCP particles, are larger than about 50 μm, preferably larger than about 100 μm. In one embodiment, the particulate material, preferably the ceramic particles of the invention, more preferably HA, β-TCP and/or HA/β-TCP particles, have a mean diameter larger than about 50 μm, preferably larger than about 100 μm.

In one embodiment, the particulate material, preferably the ceramic particles of the invention, more preferably HA, β-TCP and/or HA/β-TCP particles, have a mean diameter of at least about 50 μm, preferably of at least about 100 μm, more preferably of at least about 150 μm. In another embodiment, the particulate material, preferably the ceramic particles of the invention, more preferably HA, β-TCP and/or HA/β-TCP particles, have a mean diameter of at least about 200 μm, preferably of at least about 250 μm, more preferably of at least about 300 μm.

In another embodiment, the particulate material, preferably the ceramic particles of the invention, more preferably HA, β-TCP and/or HA/β-TCP particles, have a mean diameter of at most about 2,500 μm, preferably of at most about 2,000 μm, more preferably of at most about 1,500 μm. In one embodiment, the particulate material, preferably the ceramic particles of the invention, more preferably HA, β-TCP and/or HA/β-TCP particles, have a mean diameter of at most about 1,000 μm, 900 μm, 800 μm, 700 μm or 600 μm.

In one embodiment, the particulate material, preferably the ceramic particles of the invention, more preferably HA, β-TCP and/or HA/β-TCP particles, have a mean diameter ranging from about 50 μm to about 1,500 μm, preferably from about 50 μm to about 1,250 μm, more preferably from about 100 μm to about 1,000 μm. In one embodiment, the particulate material, preferably the ceramic particles of the invention, more preferably HA, β-TCP and/or HA/β-TCP particles, have a mean diameter ranging from about 100 μm to about 800 μm, preferably from about 150 μm to about 700 μm, more preferably from about 200 μm to about 600 μm.

In one embodiment, the HA/β-TCP particles have a mean diameter ranging from about 50 μm to about 1,500 μm, preferably from about 50 μm to about 1,250 μm, more preferably from about 100 μm to about 1,000 μm. In one embodiment, the HA and β-TCP particles have a mean diameter ranging from about 100 μm to about 800 μm, preferably from about 150 μm to about 700 μm, more preferably from about 200 μm to about 600 μm.

In practice, the measure of the mean sizes and diameters of particles may be performed by any suitable methods known in the state of the art, or a method adapted therefrom. Non-limiting examples of such methods include atomic force microscopy (AFM), transmission electron microscopy (TEM), scanning electron microscopy (SEM), dynamic light scattering (DLS).

In one embodiment, the ratio between HA and β-TCP (HA/β-TCP ratio) in the particles ranges from about 0/100 to about 100/0, preferably from about 10/90 to about 90/10, more preferably from about 20/80 to about 80/20. In one embodiment, the ratio HA/β-TCP in the particles ranges from about 30/70 to about 70/30, from about 35/65 to about 65/35, or from about 40/60 to about 60/40.

In one embodiment, the HA/β-TCP ratio in the particles is 0/100, i.e., the particles are particles of β-tricalcium phosphate. In another embodiment, the HA/β-TCP ratio in the particles is 100/0, i.e., the particles are particles of hydroxyapatite. In one embodiment, the HA/β-TCP ratio in the particles is about 10/90. In another embodiment, the HA/β-TCP ratio in the particles is about 90/10. In one embodiment, the HA/β-TCP ratio in the particles is about 20/80. In another embodiment, the HA/β-TCP ratio in the particles is about 80/20. In one embodiment, the HA/β-TCP ratio in the particles is about 30/70. In another embodiment, the HA/β-TCP ratio in the particles is about 70/30. In another embodiment, the HA/β-TCP ratio in the particles is about 35/65. In another embodiment, the HA/β-TCP ratio in the particles is about 65/35. In one embodiment, the HA/β-TCP ratio in the particles is about 40/60. In another embodiment, the HA/β-TCP ratio in the particles is about 60/40. In another embodiment, the HA/β-TCP ratio in the particles is about 50/50.

In one embodiment, the HA/β-TCP ratio in the particles is 100/0, 99/1, 98/2, 97/3, 96/4, 95/5, 94/6, 93/7, 92/8, 91/9, 90/10, 89/11, 88/12, 87/13, 86/14, 85/15, 84/16, 83/17, 82/18, 81/19, 80/20, 79/21, 78/22, 77/23, 76/24, 75/25, 74/26, 73/27, 72/28, 71/29, 70/30, 69/31, 68/32, 67/33, 66/34, 65/35, 64/36, 63/37, 62/38, 61/39, 60/40, 59/41, 58/42, 57/43, 56/44, 55/45, 54/46, 53/47, 52/48, 51/49, 50/50, 49/51, 48/52, 47/53, 46/54, 45/55, 44/56, 43/57, 42/58, 41/59, 40/60, 39/61, 38/62, 37/63, 36/64, 35/65, 34/66, 33/67, 32/68, 31/69, 30/70, 29/71, 28/72, 27/73, 26/74, 25/75, 24/76, 23/77, 22/78, 21/79, 20/80, 19/81, 18/82, 17/83, 16/84, 15/85, 14/86, 13/87, 12/88, 11/89, 10/90, 9/91, 8/92, 7/93, 6/94, 5/95, 4/96, 3/97, 2/98, 1/99, or 0/100.

According to one embodiment, the quantity of particulate material, preferably ceramic particles, more preferably HA, β-TCP and/or HA/β-TCP particles, is optimal for providing a 3D structure to the biomaterial. In one embodiment, the particulate material, preferably the ceramic particles, more preferably HA, β-TCP and/or HA/β-TCP particles, are added at a concentration ranging from about 0.1 cm³ to about 5 cm³ for a 150 cm² vessel, preferably from about 0.5 cm³ to about 3 cm³, more preferably from about 1 cm³ to about 3 cm³. In a preferred embodiment, the particulate material, preferably the ceramic particles, more preferably HA, β-TCP and/or HA/β-TCP particles, are added at a concentration of about 1.5 cm³ to about 3 cm³ for a 150 cm² vessel.

In one embodiment, the particulate material, preferably the ceramic particles, more preferably HA, β-TCP and/or HA/β-TCP particles, are added at a concentration ranging from about 7.10⁻³ to 7.10⁻² cm³ per mL of medium. In one embodiment, the particulate material, preferably the ceramic particles, more preferably HA, β-TCP and/or HA/β-TCP particles, are added at a concentration ranging from about 3.3.10⁻³ to 3.3.10⁻² cm³ per cm² of vessel.

In one embodiment, the particulate material, preferably the ceramic material, of the invention is added to the culture medium after differentiation of the cells. In one embodiment, the particulate material, preferably the ceramic material, of the invention is added when cells are sub-confluent. In one embodiment, the particulate material, preferably the ceramic material, of the invention is added when cells are overconfluent. In one embodiment, the particulate material, preferably the ceramic material, of the invention is added when cells have reached confluence after differentiation. In others words, in one embodiment, the particulate material, preferably the ceramic material, of the invention is added when cells have reached confluence in differentiation medium. In one embodiment, the particulate material, preferably the ceramic material, of the invention is added at least 5 days after P4, preferably 10 days after P4, more preferably 15 days after P4. In one embodiment, the particulate material, preferably the ceramic material, of the invention is added from 5 to 30 days after P4, preferably from 10 to 25 days after P4, more preferably from 15 to 20 days after P4.

In another embodiment, the particulate material of the invention is demineralized bone matrix (DBM).

In one embodiment, DBM is of animal origin, preferably of mammal origin, more preferably of human origin. In a particular embodiment, human DBM is obtained by grinding cortical bones from human donors.

Methods to obtain DBM are known in the art. For example, human bone tissue may first be defatted by acetone (e.g., at about 99%) bath during an overnight and then be washed in demineralized water during about 2 hours. Decalcification may be performed by immersion in HCL (e.g., at about 0.6 N) during about 3 hours (20 mL solution per gram of bone) under agitation at room temperature. Then, demineralized bone powder may be rinsed with demineralized water during about 2 hours and the pH is controlled. If the pH is too acid, DBM may be buffered with a phosphate solution (e.g., at about 0.1 M) under agitation. Finally, DBM may be dried and weighted. The DBM may be sterilized by Gamma irradiation following techniques known in the field, for example at about 25 kGray.

In one embodiment, the DBM is allogenic. In one embodiment, the DBM is homogenous. In another embodiment, the DBM is heterogeneous.

In one embodiment, DBM is in the form of particles, herein referred to as demineralized bone matrix particles or DBM particles. In one embodiment, the DBM particles have a mean diameter ranging from about 50 to about 2,500 μm, preferably from about 50 μm to about 1500 μm, more preferably from about 50 μm to about 1,000 μm. In one embodiment, the DBM particles have a mean diameter ranging from about 100 μm to about 1,500 μm, more preferably from about 150 μm to about 1,000 μm. In one embodiment, the DBM particles have a mean diameter ranging from about 200 to about 1,000 μm, preferably from about 200 μm to about 800 μm, more preferably from about 300 μm to about 700 μm.

In one embodiment, the multidimensional structure of the invention comprises an extracellular matrix. In one embodiment, the extracellular matrix of the invention derives from the differentiated cells, preferably differentiated ASCs. In one embodiment, the extracellular matrix of the invention is produced by the cells, preferably ASCs. In one embodiment, the terms “produced” and “secreted” are intended to substitute one another. As used herein, the term “extracellular matrix” (ECM) means a non-cellular three-dimensional macromolecular network. Matrix components of ECM bind to each other as well as cell adhesion receptors, thereby forming a complex network into which cells reside in tissues or in multidimensional structure according to the invention.

In one embodiment, the extracellular matrix of the invention comprises collagen, proteoglycans/glycosaminoglycans, elastin, fibronectin, laminin, and/or other glycoproteins. In a particular embodiment, the extracellular matrix of the invention comprises collagen. In another particular embodiment, the extracellular matrix of the invention comprises proteoglycans. In another particular embodiment, the extracellular matrix of the invention comprises collagen and proteoglycans. In one embodiment, the extracellular matrix of the invention comprises growth factors, proteoglycans, secreting factors, extracellular matrix regulators, and glycoproteins.

In one embodiment, the cells, preferably ASCs, and the particulate material, preferably the gelatin, the DBM or the ceramic material, of the invention are embedded into the extracellular matrix.

In certain embodiments, step 1) is performed in the presence of one or more exogenous factors selected in the group comprising growth factors, transcription factors, osteogenic factors, activators and/or inhibitors of signal pathways, and a mixture thereof.

As used herein, growth factors are intended to refer to polypeptides that regulate many aspects of cellular function, including survival, proliferation, migration and differentiation. In some embodiments, the growth factors according to the invention include, but are not limited to, BMPs, EGF, FGFs, HGF, IGF-1, OPG, SDF-1α, TGFB-1, TGFB-3, VEGFA and VEFGB. In certain embodiments, the growth factors according to the invention include, but are not limited to, IGF-1, TGFB-1, TGFB-3, VEGFA and VEFGB.

As used herein, transcription factors are intended to refer to polypeptides that control whether a given gene is to be transcribed into its corresponding RNA. In some embodiments, the transcription factors according to the invention include, but are not limited to, AKT, ANG, ANGPT1, ANGPTL4, ANPEP, COL18A1, CTGF,CXCL1, EDN1, EFNA1, EFNB2, ENG, EPHB4, F3, FGF1, FGF2, FN1, HIF1A, ID1, IL6, ITGAV, JAG1, LEP, MMP14, MMP2, NRP1, PTGS1, SERPINE1, SERPINF1, TGFB1, TGFBR1, THBS1, THBS2, TIMP1, TIMP2, TIMP3, VEGFA, VEGFB, VEGFC.

In certain embodiments, the transcription factors according to the invention include, but are not limited to, SMAD-2, SMAD-3, SMAD-4, SMAD-5.

As used herein, osteogenic factors are intended to refer to polypeptides that promote osteogenesis and/or impair osteoclasia. In some embodiments, the osteogenic factors according to the invention are involve in the skeletal development. Non-limitative examples of osteogenic factors according to the invention include OPG, SDF-1α, BMPR-1A, BMP-2, FGFR-1, FGFR-2, TWIST-1, CSF-1, IGFR, RUNX2, TGFBR-1.

In practice, under the suitable culture conditions, the cells are secreting an extracellular matrix and synthesize polypeptides and nucleic acids that promote tissue regeneration and/or tissue repair, in particular promote osteogenesis and/or chondrogenesis. Said polypeptides and nucleic acids may be considered as being biomarkers for the tissue regeneration and/or tissue repair, including osteogenesis and/or chondrogenesis properties, and may be monitored at the polypeptide level and/or at the nucleic acids, by the means of methods mentioned hereinabove.

In practice, the content of factors, as polypeptides, of the composition according to the instant invention may be assessed by any suitable method known in the art, or any method adapted therefrom. Illustratively, the expression or absence of expression (non-expression) of these biomarkers may be monitored at the nucleic acid level or the polypeptide level. Non-limitative example of methods for monitoring biomarkers at the nucleic acid level encompasses RT-PCR (qPCR) analysis of RNA extracted from cultured cells with specific primers. Non-limitative examples of methods for monitoring biomarkers at the polypeptide level encompass immunofluorescence analysis with markers-specific antibodies, such as Western blotting or ELISA; Fluorescent activated cell sorting (FACS); and enzymatic assays.

In some embodiments, the method for producing a composition according to the invention further comprises the step(s) of:
3) purifying the RNAs extracted at step 2); and optionally
4) freeze-drying the purified RNAs obtained at step 3).

In certain embodiments, the miRNAs content includes cellular miRNAs and/or exosomes-derived miRNAs.

In some embodiments, at least part of the miRNAs is cellular. In practice, cellular miRNAs may be isolated by any suitable method known from the state of the art, or a method adapted therefrom. One may refer, e.g., to Chapter 7: Extraction, Purification, and Analysis of mRNA from Eukaryotic Cells of Molecular Cloning: a laboratory manual (Russell and Sambrook; 2001; Cold Spring Harbor Laboratory). Illustratively, miRNAs may be isolated by a commercial kit, such as, e.g., RNeasy Mini kit (Qiagen®) or MagMax mirVana Total RNA isolation kit (Applied Biosystems®).

In one embodiment, the cellular miRNAs are selected in a group comprising hsa-let-7a-5p, hsa-miR-210-3p, hsa-miR-29b-3p, hsa-miR-30e-3p, hsa-let-7b-5p, hsa-miR-3184-3p, hsa-miR-92a-3p, hsa-miR-320a, hsa-miR-24-3p, hsa-let-7d-5p, hsa-miR-193b-5p, hsa-miR-361-3p, hsa-miR-199a-5p, hsa-miR-25-3p, hsa-miR-181a-5p, hsa-miR-151a-3p, hsa-miR-214-3p, hsa-miR-193a-5p, hsa-miR-30c-5p, hsa-miR-154-5p, hsa-let-7f-5p, hsa-miR-199a-3p, hsa-miR-664b-3p, hsa-miR-664a-5p, hsa-miR-3607-5p, hsa-miR-29a-3p, hsa-miR-27a-3p, hsa-miR-92b-3p, hsa-miR-199b-3p, hsa-miR-342-3p, hsa-miR-320b, hsa-miR-1291, hsa-let-7e-5p, hsa-miR-130a-3p, hsa-miR-3651, hsa-miR-103b, hsa-miR-1273g-3p, hsa-miR-30a-3p, hsa-miR-664b-5p, hsa-miR-34a-3p, hsa-miR-125a-5p, hsa-miR-145-5p, hsa-miR-664a-3p, hsa-miR-140-5p, hsa-miR-21-5p, hsa-miR-28-3p, hsa-miR-98-5p, hsa-miR-3609, hsa-let-7i-5p, hsa-miR-93-5p, hsa-miR-146b-5p, hsa-miR-374c-3p, hsa-miR-125b-5p, hsa-miR-34a-5p, hsa-miR-337-3p, hsa-miR-10a-5p, hsa-let-7g-5p, hsa-miR-222-3p, hsa-miR-4449, hsa-miR-22-3p, hsa-miR-191-5p, hsa-miR-3074-5p, hsa-miR-6516-3p, hsa-miR-4668-5p, hsa-miR-574-3p, hsa-miR-424-5p, hsa-let-7i-3p, hsa-miR-24-2-5p, hsa-miR-199b-5p, hsa-miR-424-3p, hsa-miR-103a-3p, hsa-miR-29b-1-5p, hsa-miR-423-5p, hsa-miR-328-3p, hsa-miR-324-5p, hsa-miR-335-5p, hsa-miR-574-5p, hsa-miR-17-5p, hsa-miR-660-5p, hsa-miR-425-5p, hsa-miR-23b-3p, hsa-miR-23a-3p, hsa-miR-185-5p, hsa-miR-4461, hsa-miR-196a-5p, hsa-let-7d-3p, hsa-miR-374b-5p, hsa-miR-127-3p, hsa-let-7c-5p, hsa-miR-423-3p, hsa-miR-409-3p, hsa-miR-196b-5p, hsa-miR-221-3p, hsa-miR-382-5p, hsa-miR-619-5p, hsa-miR-3613-5p, hsa-miR-3653-5p, hsa-miR-19b-3p, hsa-miR-99b-5p, hsa-miR-376c-3p, hsa-miR-99b-3p, hsa-miR-663b, hsa-miR-495-3p, hsa-miR-454-3p, and a combination thereof.

In one embodiment, the cellular miRNAs are selected in a group comprising hsa-let-7a-5p, hsa-let-7b-5p, hsa-miR-24-3p, hsa-let-7f-5p, hsa-miR-199a-5p, hsa-miR-214-3p, hsa-miR-3607-5p, hsa-miR-125a-5p, hsa-miR-199b-3p, hsa-miR-125b-5p, hsa-miR-21-5p, hsa-let-7e-5p, hsa-let-7i-5p, hsa-let-7g-5p, hsa-miR-574-3p, hsa-miR-574-5p, hsa-miR-191-5p, hsa-miR-196a-5p, hsa-miR-221-3p, hsa-miR-25-3p, hsa-miR-423-5p, hsa-miR-210-3p, hsa-miR-1273g-3p, hsa-let-7d-5p, hsa-miR-199b-5p, hsa-miR-199a-3p, hsa-miR-193 a-5p, hsa-miR-3184-3p, hsa-miR-3653-5p, hsa-miR-342-3p, hsa-miR-28-3p, hsa-miR-23b-3p, hsa-let-7c-5p, hsa-miR-222-3p, hsa-miR-29a-3p, hsa-miR-92a-3p, hsa-miR-30a-3p, hsa-miR-424-3p, hsa-miR-423-3p, hsa-miR-34a-5p, hsa-miR-424-5p, hsa-miR-145-5p, hsa-miR-328-3p, hsa-miR-3074-5p, hsa-let-7d-3p, hsa-miR-93-5p, hsa-miR-23a-3p, hsa-miR-19b-3p, hsa-miR-146b-5p, hsa-miR-3 20b, hsa-miR-337-3p, hsa-miR-17-5p, hsa-miR-130a-3p, hsa-miR-193b-5p, hsa-miR-382-5p, hsa-miR-30c-5p, hsa-miR-98-5p, hsa-miR-664a-3p, hsa-miR-92b-3p, hsa-miR-4449, hsa-miR-320a, hsa-miR-181a-5p, hsa-miR-3651, hsa-miR-185-5p, hsa-miR-664b-5p, hsa-miR-196b-5p, hsa-miR-27a-3p, hsa-miR-29b-3p, hsa-miR-664b-3p, hsa-miR-99b-5p, hsa-miR-103a-3p, hsa-miR-6516-3p, hsa-miR-22-3p, hsa-miR-26a-5p, hsa-miR-103b, hsa-miR-1291, hsa-miR-425-5p, hsa-miR-22-5p, hsa-miR-374c-3p, hsa-let-7i-3p, hsa-miR-374b-5p, hsa-miR-455-3p, hsa-miR-532-3p, hsa-miR-619-5p, hsa-miR-28-5p, hsa-miR-10a-5p, hsa-miR-151a-3p, hsa-miR-30e-3p, hsa-miR-324-5p, hsa-miR-495-3p, hsa-miR-576-5p, hsa-miR-625-3p, hsa-miR-671-5p, hsa-miR-1271-5p, hsa-miR-186-5p, hsa-miR-23a-5p, hsa-miR-3613-5p, hsa-miR-376c-3p, hsa-miR-409-3p, hsa-miR-4461, hsa-miR-454-3p, hsa-miR-6724-5p, hsa-let-7b-3p, hsa-miR-190a-5p, hsa-miR-26b-3p, hsa-miR-3609, hsa-miR-411-5p, hsa-miR-425-3p, hsa-miR-4485-3p and a mixture thereof.

In one aspect, the invention relates to a pharmaceutical composition comprising cellular miRNAs obtained from a culture of differentiated cells in the presence of a particulate material, wherein the cells and the particulate material were embedded in an extracellular matrix. In some embodiments, the pharmaceutical composition comprises cellular miRNAs obtained from a culture of osteo-differentiated cells in the presence of a particulate material, wherein the cells and the particulate material were embedded in an extracellular matrix. In certain embodiments, the pharmaceutical composition comprises cellular miRNAs obtained from a culture of osteo-differentiated MSCs, in particular osteo-differentiated ASCs, in the presence of a particulate material, wherein the cells and the particulate material were embedded in an extracellular matrix. In some embodiments, the pharmaceutical composition comprises cellular miRNAs obtained from a culture of osteo-differentiated ASCs, in the presence of a gelatin, wherein the cells and the gelatin were embedded in an extracellular matrix. In certain embodiments, the pharmaceutical composition comprises cellular miRNAs obtained from a culture of osteo-differentiated ASCs, in the presence of a ceramic material, wherein the cells and the ceramic material were embedded in an extracellular matrix.

In certain embodiments, at least part of the miRNAs is secreted by the cells, preferably in the form of exosomes or exosome-like vesicles. In said embodiments, at least part of the miRNAs content is comprised in exosomes or exosome-like vesicles. In some embodiments, at least part of the miRNAs content is exosomal miRNAs.

As used herein, the term “exosome” refers to endocytic-derived nanovesicles that are secreted by nearly all cell types in the body. The exosomes comprise proteins, nucleic acids, in particular miRNAs, and lipids. In practice, the exosomes may be isolated and/or purified according to any suitable method known in the state of the art, or a method adapted therefrom. Illustratively, the exosome fraction may be isolated by differential centrifugation from culture medium; by polymer precipitation; by high-performance liquid chromatography (HPLC). Non-limitative example of differential centrifugation method from culture medium may include the following steps:
Centrifugation for 10-20 min at a speed of about 300×g to about 500×g, so as to remove cells;
Centrifugation for 10-20 min at a speed of about 1,500×g to about 3,000×g, so as to remove dead cells;
Centrifugation for 20-45 min at a speed of about 7,500×g to about 15,000×g, so as to remove cell debris;
One or more ultracentrifugation for 30-120 min at a speed of about 100,000×g to about 200,000×g, so as to pellet the exosomes.

Alternative methods to isolate exosomes may take advantage of commercial kits, such as, e.g., the exoEasy Maxi Kit (Qiagen®) or the Total Exosome Isolation Kit (ThermoFisher Scientific®).

In some embodiments, the exosomes or the exosome-like vesicles have an average diameter ranging from about 25 nm to about 150 nm, preferably from about 30 nm to 120 nm. Within the scope of the instant invention, the expression “from about 25 nm to about 150 nm” includes 25, 30, 35, 40, 45, 50, 55, 60, 65, 70, 75, 80, 85, 90, 95, 100, 105, 110, 115, 120, 125, 130, 135, 140, 145 and 150 nm.

In one embodiment, the exosomal miRNAs are selected in a group comprising hsa-let-7a-5p, hsa-miR-92a-3p, hsa-miR-92b-3p, hsa-miR-24-2-5p, hsa-let-7b-5p, hsa-miR-125b-5p, hsa-miR-335-5p, hsa-miR-26a-2-3p, hsa-let-7f-5p, hsa-miR-337-3p, hsa-let-7f-1-3p, hsa-miR-301 a-3p, hsa-miR-24-3p, hsa-miR-93-5p, hsa-miR-196b-5p, hsa-miR-98-3p, hsa-miR-21-5p, hsa-miR-409-3p, hsa-miR-3613-3p, hsa-miR-1273a, hsa-miR-23b-3p, hsa-miR-199a-3p, hsa-miR-23a-5p, hsa-miR-28-5p, hsa-miR-1273g-3p, hsa-miR-145-5p, hsa-miR-374b-5p, hsa-miR-34a-3p, hsa-miR-574-3p, hsa-miR-30a-3p, hsa-miR-660-5p, hsa-miR-425-3p, hsa-miR-25-3p, hsa-miR-382-5p, hsa-miR-186-5p, hsa-miR-505-3p, hsa-let-7e-5p, hsa-miR-19b-3p, hsa-miR-454-3p, hsa-miR-34b-3p, hsa-miR-214-3p, hsa-miR-210-3p, hsa-miR-10a-5p, hsa-miR-361-3p, hsa-miR-199a-5p, hsa-miR-619-5p, hsa-miR-495-3p, hsa-miR-10b-5p, hsa-miR-196a-5p, hsa-miR-17-5p, hsa-miR-425-5p, hsa-miR-1306-5p, hsa-miR-199b-5p, hsa-miR-193a-5p, hsa-miR-2053, hsa-miR-22-5p, hsa-miR-221-3p, hsa-miR-320b, hsa-miR-5096, hsa-miR-378a-3p, hsa-miR-424-5p, hsa-miR-193b-5p, hsa-miR-494-3p, hsa-miR-411-5p, hsa-miR-23a-3p, hsa-miR-320a, hsa-miR-27a-3p, hsa-miR-505-5p, hsa-let-7c-5p, hsa-miR-151a-3p, hsa-miR-4449, hsa-miR-664a-3p, hsa-miR-199b-3p, hsa-let-7a-3p, hsa-miR-532-3p, hsa-miR-26a-5p, hsa-miR-191-5p, hsa-miR-30e-3p, hsa-miR-532-5p, hsa-miR-377-3p, hsa-miR-574-5p, hsa-miR-22-3p, hsa-miR-126-5p, hsa-miR-485-3p, hsa-miR-424-3p, hsa-miR-99b-5p, hsa-miR-30c-5p, hsa-miR-590-3p, hsa-miR-423-5p, hsa-miR-625-3p, hsa-miR-130b-3p, hsa-miR-99a-3p, hsa-miR-342-3p, hsa-miR-4668-5p, hsa-miR-136-3p, hsa-miR-143-3p, hsa-let-7d-3p, hsa-miR-29b-3p, hsa-miR-15b-3p, hsa-miR-26b-3p, hsa-miR-130a-3p, hsa-miR-423-3p, hsa-miR-29b-1-5p, hsa-miR-3607-5p, hsa-miR-3184-3p, hsa-miR-376c-3p, hsa-miR-99b-3p, hsa-miR-3651, hsa-miR-222-3p, hsa-let-7b-3p, hsa-miR-127-3p, hsa-miR-374a-3p, hsa-let-7g-5p, hsa-miR-3074-5p, hsa-miR-134-5p, hsa-miR-376a-3p, hsa-miR-125a-5p, hsa-miR-98-5p, hsa-miR-324-5p, hsa-miR-485-5p, hsa-let-7d-5p, hsa-miR-185-5p, hsa-miR-3605-3p, hsa-miR-103b, hsa-miR-29a-3p, hsa-miR-19a-3p, hsa-miR-101-3p, hsa-miR-126-3p, hsa-let-7i-5p, hsa-miR-34a-5p, hsa-miR-103a-3p, hsa-miR-149-5p, hsa-miR-146b-5p, hsa-miR-374c-3p, hsa-miR-1246, hsa-miR-193b-3p, hsa-miR-4454, hsa-miR-181a-5p, hsa-miR-138-5p, hsa-miR-223-3p, hsa-miR-28-3p, hsa-miR-328-3p, hsa-miR-190a-5p, hsa-miR-340-3p, hsa-miR-874-3p, hsa-miR-7847-3p, hsa-miR-6724-5p, hsa-miR-369-5p, and a combination thereof.

In one embodiment, the exosomal miRNAs are selected in a group comprising hsa-let-7a-5p, hsa-let-7b-5p, hsa-miR-24-3p, hsa-miR-21-5p, hsa-let-7f-5p, hsa-miR-574-3p, hsa-miR-23b-3p, hsa-miR-1273g-3p, hsa-miR-25-3p, hsa-miR-199a-5p, hsa-miR-196a-5p, hsa-miR-214-3p, hsa-miR-125a-5p, hsa-miR-221-3p, hsa-miR-222-3p, hsa-let-7e-5p, hsa-miR-191-5p, hsa-miR-199b-3p, hsa-miR-342-3p, hsa-miR-23 a-3p, hsa-miR-424-3p, hsa-miR-28-3p, hsa-let-7g-5p, hsa-miR-92a-3p, hsa-miR-424-5p, hsa-let-7d-3p, hsa-miR-4454, hsa-miR-146b-5p, hsa-miR-423-5p, hsa-miR-29a-3p, hsa-miR-574-5p, hsa-miR-199b-5p, hsa-miR-125b-5p, hsa-miR-3184-3p, hsa-let-7c-5p, hsa-miR-337-3p, hsa-let-7d-5p, hsa-miR-145-5p, hsa-miR-93-5p, hsa-miR-619-5p, hsa-miR-130a-3p, hsa-let-7i-5p, hsa-miR-409-3p, hsa-miR-210-3p, hsa-miR-199a-3p, hsa-miR-30a-3p, hsa-miR-320b, hsa-miR-193a-5p, hsa-miR-382-5p, hsa-miR-423-3p, hsa-miR-17-5p, hsa-miR-19b-3p, hsa-miR-92b-3p, hsa-miR-320a, hsa-miR-3074-5p, hsa-miR-376c-3p, hsa-let-7b-3p, hsa-miR-625-3p, hsa-miR-99b-5p, hsa-miR-34a-5p, hsa-miR-5096, hsa-miR-30e-3p, hsa-miR-22-3p, hsa-miR-151a-3p, hsa-miR-186-5p, hsa-miR-193b-5p, hsa-miR-328-3p, hsa-miR-4449, hsa-miR-27a-3p, hsa-miR-30c-5p, hsa-miR-494-3p, hsa-miR-98-5p, hsa-miR-10a-5p, hsa-miR-29b-3p, hsa-miR-374b-5p, hsa-miR-335-5p, hsa-miR-374c-3p, hsa-miR-425-5p, hsa-miR-181a-5p, hsa-miR-196b-5p, hsa-let-7f-1-3p, hsa-miR-4668-5p, hsa-miR-660-5p, hsa-miR-664a-3p, hsa-miR-185-5p, hsa-miR-3651, hsa-miR-495-3p, hsa-let-7a-3p, hsa-miR-28-5p, hsa-miR-99b-3p, hsa-miR-103a-3p, hsa-miR-19a-3p, hsa-miR-126-5p, hsa-miR-2053, hsa-miR-29b-1-5p, hsa-miR-3648, hsa-miR-374a-3p, hsa-miR-454-3p, hsa-miR-532-3p, hsa-miR-136-3p, hsa-miR-361-3p, hsa-miR-1246, hsa-miR-130b-3p, hsa-miR-134-5p, hsa-miR-154-5p, hsa-miR-34a-3p, hsa-miR-576-5p, hsa-miR-874-3p, hsa-miR-100-5p, hsa-miR-103b, hsa-miR-1273a, hsa-miR-1306-5p, hsa-miR-138-5p, hsa-miR-15b-3p, hsa-miR-26b-3p, hsa-miR-10b-5p, hsa-miR-22-5p, hsa-miR-3613-3p, hsa-miR-655-3p, hsa-miR-7-1-3p, hsa-miR-23a-5p, hsa-miR-24-2-5p, hsa-miR-3605-3p, hsa-miR-6832-3p, hsa-miR-146a-5p, hsa-miR-16-2-3p, hsa-miR-181b-5p, hsa-miR-26a-2-3p, hsa-miR-376a-3p, hsa-miR-539-5p, hsa-miR-708-5p, hsa-miR-98-3p, hsa-miR-1237-5p, hsa-miR-223-3p, hsa-miR-532-5p, hsa-miR-542-3p, hsa-miR-663a, hsa-miR-101-3p, hsa-miR-143-3p, hsa-miR-21-3p, hsa-miR-224-5p, hsa-miR-26a-5p, hsa-miR-27a-5p, hsa-miR-324-5p, hsa-miR-340-3p, hsa-miR-379-5p, hsa-miR-409-5p, hsa-miR-543, hsa-miR-5787, hsa-miR-6089, hsa-miR-127-3p, hsa-miR-149-5p, hsa-miR-181c-5p, hsa-miR-193b-3p, hsa-miR-222-5p, hsa-miR-3613-5p, hsa-miR-365b-3p, hsa-miR-3960, hsa-miR-485-3p, hsa-miR-6087, hsa-miR-92a-1-5p and a mixture thereof.

In one aspect, the invention relates to a pharmaceutical composition comprising exosomes obtained from a culture of differentiated cells in the presence of a particulate material, wherein the cells and the particulate material were embedded in an extracellular matrix. In some embodiments, the pharmaceutical composition comprises exosomes obtained from a culture of osteo-differentiated cells in the presence of a particulate material, wherein the cells and the particulate material were embedded in an extracellular matrix. In certain embodiments, the pharmaceutical composition comprises exosomes obtained from a culture of osteo-differentiated MSCs, in particular osteo-differentiated ASCs, in the presence of a particulate material, wherein the cells and the particulate material were embedded in an extracellular matrix. In some embodiments, the pharmaceutical composition comprises exosomes obtained from a culture of osteo-differentiated ASCs, in the presence of a gelatin, wherein the cells and the gelatin were embedded in an extracellular matrix. In certain embodiments, the pharmaceutical composition comprises exosomes obtained from a culture of osteo-differentiated ASCs, in the presence of a ceramic material, wherein the cells and the ceramic material were embedded in an extracellular matrix.

Another aspect of the invention pertains to a composition comprising at least three miRNAs selected in any one of Table 1, Table 2, Table 3, Table 4, Table 5, Table 6, Table 7, Table 8, Table 9, Table 10, Table 11 or Table 12, obtainable by a method according to the invention.

In another aspect, the invention also relates to a composition for use as a medicament, in particular for the prevention and/or the treatment of a tissue disorder.

In another aspect, the invention also relates to a composition for use as a medicament, in particular for the prevention and/or the treatment of a bone disorder and/or a cartilage disorder.

The invention also pertains to a kit for the prevention and/or the treatment of a tissue disorder comprising a pharmaceutical composition according to the invention and a means to administer said pharmaceutical composition.

The invention also pertains to a kit for the prevention and/or the treatment of a bone disorder and/or a cartilage disorder comprising a pharmaceutical composition according to the invention and a means to administer said pharmaceutical composition.

In some embodiments, the means to administer the pharmaceutical composition according to the invention include, but are not limited to, syringes, catheters, trocars, patches, dressings, spatulas, cups, nebulizers, and the likes.

In some embodiments, the kit further comprises one or more additional active ingredients for the prevention and/or the treatment of a tissue disorder. In some embodiments, the kit further comprises one or more additional active ingredients for the prevention and/or the treatment of a bone disorder and/or a cartilage disorder. Non-limitative examples of such active ingredients may be growth factors, transcription factors, osteogenic factors, anti-cancer agents, antibiotics, immunotherapeutic agents, chemotherapeutic agents, and the likes.

In some embodiments, the one or more additional active ingredient(s) is/are intended to be administered prior, concomitantly or upon the administration of the pharmaceutical composition according to the invention.

### BRIEF DESCRIPTION OF THE DRAWINGS

FIG. 1: Plots showing the proliferation of HDFa (express as viability (DO)) in the absence (curve 1) or the presence of 2.5 μg/ml (curve 2) and 25 μg/ml (curve 3) of NVD002-exosomes under normoxia (21% O₂).

FIG. 2: Plots showing the linear regression of the progression speed of HDFa in the absence (curve 1) or the presence of 2.5 μg/ml (curve 2) and 25 μg/ml (curve 3) of NVD002-exosomes calculated from FIG. 1. Results are expressed as the percentage of viable cells vs negative control (without exosomes) at 24 h and 32 h. ^{∘ ∘}: statistical difference between negative control and 2.5 μg/ml.

FIG. 3: Plots showing the proliferation of HDFa (express as viability (DO)) in the absence (curve 1) or the presence of 2.5 μg/ml (curve 2) and 25 μg/ml (curve 3) of NVD002-exosomes under hypoxia (1% O₂).

FIG. 4: Plots showing the linear regression of the progression speed of HDFa in the absence (curve 1) or the presence of 2.5 μg/ml (curve 2) and 25 μg/ml (curve 3) of NVD002-exosomes calculated from FIG. 3. Results are expressed as the percentage of viable cells vs negative control (without exosomes) at 32 h and 48 h. ***, ****: statistical difference between negative control and 2.5 μg/ml.

FIG. 5: Plot showing the effect of exosomes on RANKL mediated osteoclast formation. Osteoclast precursors (CD14 19-10) were cultured in medium supplemented with 1% FBS, 25 ng/mL human MCSF, +/−100 ng/mL human RANKL, and +/−exosomes. TRAP staining was performed on D8. The statistical significance of the difference between the treatments and the Plus RANKL control was determined using the Mann Whitney test (Statview software). **: p<0.01; ***: p<0.005.

FIG. 6: Plot showing the effect of exosomes on osteoclast viability. Mature osteoclasts were differentiated from CD14+ cells (CD14 19-10) and cultured in medium supplemented with 1% FBS, 25 ng/mL human MCSF, 100 ng/mL human RANKL, +/−exosomes for 48 hours. TRAP staining was performed 48 hours after the addition of the treatment. The statistical significance of the difference between the treatments and the Plus RANKL control was determined using the Mann Whitney test (Statview software).

FIG. 7A-Q: Plots showing the osteogenic and angiogenic genes expression in different culture conditions. (MD) represents the differentiation medium; (MD+SCL) the differentiation medium in the presence of sclerotin; (MD+SCL+Exosomes) represents the differentiation medium in the presence of sclerotin and of NVD003-derived exosomes.

FIG. 8A-Q: Plots showing the osteogenic and angiogenic genes expression in different culture conditions, i.e., in the presence of 10 μg/ml, 25 μg/ml or 75 μg/ml of NVD003-derived exosomes. Horizontal line shows the basal expression level in cells in proliferation medium. C+ shows the expression level in cells in osteo-differentiation medium.

FIG. 9 is a plot showing the proliferation of human osteosarcoma cells H143B in the absence (black curve) or in the presence of 2.5 μg/ml (dark grey curve) and 25 μg/ml (light grey curve) of NVD002-Exosomes. The proliferation is expressed as viability (DO) with respect of the time of the co-culture of the cells and the exosomes.

FIG. 10 is a plot showing the linear regression of the loss of proliferation of human osteosarcoma cells H143B in the absence (black curve) or in the presence of 2.5 μg/ml (dark grey curve) and 25 μg/ml (light grey curve) of NVD002-Exosomes. Results are expressed as the % of viable cells vs negative control (without exosomes) at each time point. **: p<0.01; ***: p<0.005; ****: p<0.0001; −: no statistical difference.

FIG. 11 is a plot showing the proliferation of human osteosarcoma cells H143B in the absence (black curve) or in the presence of 2.5 μg/ml (dark grey curve) and 25 μg/ml (light grey curve) of NVD003-Exosomes. The proliferation is expressed as viability (DO) with respect of the time of the co-culture of the cells and the exosomes.

FIG. 12 is a plot showing the linear regression of the loss of proliferation of human osteosarcoma cells H143B in the absence (black curve) or in the presence of 2.5 μg/ml (dark grey curve) and 25 μg/ml (light grey curve) of NVD003-Exosomes. Results are expressed as the % of viable cells vs negative control (without exosomes) at each time point. (*: p<0.05; **: p<0.01; −: no statistical difference.

FIG. 13 is a plot showing the proliferation of human melanoma cells A375 in the absence (black curve) or in the presence of 2.5 μg/ml (dark grey curve) and 25 μg/ml (light grey curve) of NVD002-Exosomes. The proliferation is expressed as viability (DO) with respect of the time of the co-culture of the cells and the exosomes.

FIG. 14 is a plot showing the linear regression of the loss of proliferation of human melanoma cells A375 in the absence (black curve) or in the presence of 2.5 μg/ml (dark grey curve) and 25 μg/ml (light grey curve) of NVD002-Exosomes. Results are expressed as the % of viable cells vs negative control (without exosomes) at each time point. *: p<0.05; **: p<0.01; ****: p<0.0001; ^{∘ ∘}: p<0.01; ^{∘ ∘ ∘ ∘}: p<0.0001; −: no statistical difference.

FIG. 15 is a plot showing the proliferation of human melanoma cells A375 in the absence (black curve) or in the presence of 2.5 μg/ml (dark grey curve) and 25 μg/ml (light grey curve) of NVD003-Exosomes. The proliferation is expressed as viability (DO) with respect of the time of the co-culture of the cells and the exosomes.

FIG. 16 is a plot showing the linear regression of the loss of proliferation of human melanoma cells A375 in the absence (black curve) or in the presence of 2.5 μg/ml (dark grey curve) and 25 μg/ml (light grey curve) of NVD003-Exosomes. Results are expressed as the % of viable cells vs negative control (without exosomes) at each time point. ***: p<0.005; ****: p<0.0001; ^{∘ ∘ ∘ ∘}: p<0.0001; −: no statistical difference.

FIG. 17 is a plot showing the proliferation of human glioblastoma cells U87 in the absence (black curve) or in the presence of 2.5 μg/ml (dark grey curve) and 25 μg/ml (light grey curve) of NVD002-Exosomes. The proliferation is expressed as viability (DO) with respect of the time of the co-culture of the cells and the exosomes.

FIG. 18 is a plot showing the linear regression of the loss of proliferation of human glioblastoma cells U87 in the absence (black curve) or in the presence of 2.5 μg/ml (dark grey curve) and 25 μg/ml (light grey curve) of NVD002-Exosomes. Results are expressed as the % of viable cells vs negative control (without exosomes) at each time point. *: p<0.05; ****: p<0.0001; ^{∘ ∘ ∘ ∘}: p<0.0001; −: no statistical difference.

FIG. 19 is a plot showing the proliferation of human glioblastoma cells U87 in the absence (black curve) or in the presence of 2.5 μg/ml (dark grey curve) and 25 μg/ml (light grey curve) of NVD003-Exosomes. The proliferation is expressed as viability (DO) with respect of the time of the co-culture of the cells and the exosomes.

FIG. 20 is a plot showing the linear regression of the loss of proliferation of human glioblastoma cells U87 in the absence (black curve) or in the presence of 2.5 μg/ml (dark grey curve) and 25 μg/ml (light grey curve) of NVD003-Exosomes. Results are expressed as the % of viable cells vs negative control (without exosomes) at each time point. ***: p<0.005; ****: p<0.0001; ^{∘ ∘ ∘ ∘}: p<0.0001; −: no statistical difference.

### EXAMPLES

The present invention is further illustrated by the following examples.

### Example 1

### Production of Exosomal miRNAs (miRNA Cocktails) According to the Invention

1. Materials and Methods

1.1 Isolation of hASCs

Human subcutaneous adipose tissues were harvested by lipo-aspiration following Coleman technique in the abdominal region and after informed consent and serologic screening.

Human adipose tissue-derived stem cells (hASCs) were promptly isolated from the incoming adipose tissue. Lipoaspirate can be stored at +4° C. for 24 to 72 hours or for a longer time at −18° C.

First, a fraction of the lipoaspirate was isolated for quality control purposes and the remaining volume of the lipoaspirate was measured. Then, the lipoaspirate was digested by a collagenase solution (NB 1, Serva Electrophoresis® GmbH, Heidelberg, Germany) prepared in HBSS (with a final concentration of ˜8 U/mL). The volume of the enzyme solution used for the digestion was the double of the volume of the adipose tissue. The digestion was performed during 50-70 min at 37° C.±1° C. A first intermittent shaking was performed after 15-25 min and a second one after 35-45 min. The digestion was stopped by the addition of MP medium (proliferation medium, or growth medium). The MP medium comprised DMEM medium (4.5 g/L glucose and 4 mM Ala-Gln; Sartorius Stedim Biotech®, Gottingen, Germany) supplemented with 5% human platelet lysate (hPL) (v/v). DMEM is a standard culture medium containing salts, amino acids, vitamins, pyruvate and glucose, buffered with a carbonate buffer and has a physiological pH (7.2-7.4). The DMEM used contained Ala-Gln. Human platelet lysate (hPL) is a rich source of growth factor used to stimulate in vitro growth of mesenchymal stem cells (such as hASCs).

The digested adipose tissue was centrifuged (500×g, 10 min, 20° C.) and the supernatant was removed. The pelleted Stromal Vascular Fraction (SVF) was re-suspended into MP medium and passed through a 200-500 μm mesh filter. The filtered cell suspension was centrifuged a second time (500×g, 10 min, 20° C.). The pellet containing the hASCs was re-suspended into MP medium. A small fraction of the cell suspension can be kept for cell counting and the entire remaining cell suspension was used to seed one 75 cm² T-flask (referred as Passage P0). Cell counting was performed (for information only) in order to estimate the number of seeded cells.

The day after the isolation step (day 1), the growth medium was removed from the 75 cm² T-flask. Cells were rinsed three times with phosphate buffer and freshly prepared MP medium was then added to the flask.

1.2 Growth and Expansion of Human Adipose Tissue-Derived Stem Cells

During the proliferation phase, hASCs were passaged 4 times (P1, P2, P3 and P4) in order to obtain a sufficient amount of cells for the subsequent steps of the process.

Between P0 and the fourth passage (P4), cells were cultivated on T-flasks and fed with fresh MP medium. Cells were passaged when reaching a confluence ≥70% and ≤100% (target confluence: 80-90%). All the cell culture recipients from 1 batch were passaged at the same time. At each passage, cells were detached from their culture vessel with TrypLE (Select 1×; 9 mL for 75 cm² flasks or 12 mL for 150 cm² flasks), a recombinant animal-free cell-dissociation enzyme. TrypLe digestion was performed for 5-15 min at 37° C.±2° C. and stopped by the addition of MP medium.

Cells were then centrifuged (500×g, 5 min, 20° C.), and re-suspended in MP medium. Harvested cells were pooled in order to guaranty a homogenous cell suspension. After resuspension, cells were counted. At passages P1, P2 and P3, the remaining cell suspension was then diluted to the appropriate cell density in MP medium and seeded on larger tissue culture surfaces. At these steps, 75 cm² flasks were seeded with a cell suspension volume of 15 mL, while 150 cm² flasks were seeded with a cell suspension volume of 30 mL. At each passage, cells were seeded between 0.5×10⁴ and 0.8×10⁴ cells/cm². Between the different passages, culture medium was exchanged every 3-4 days. The cell behavior and growth rate from one donor to another could slightly differ. Hence the duration between two passages and the number of medium exchanges between passages may vary from one donor to another.

1.3 Osteogenic Differentiation

At passage P4 (i.e., the fourth passage), cells were centrifuged a second time, and re-suspended in MD medium (differentiation medium). After resuspension, cells were counted a second time before being diluted to the appropriate cell density in MD medium, and a cell suspension volume of 70 mL was seeded on 150 cm² flasks and fed with osteogenic MD medium. According to this method, cells were directly cultured in osteogenic MD medium after the fourth passage. Therefore, osteogenic MD medium was added while cells have not reached confluence.

The osteogenic MD medium was composed of proliferation medium (DMEM, Ala-Gln, hPL 5%) supplemented with dexamethasone (1 μM), ascorbic acid (0.25 mM) and sodium phosphate (2.93 mM).

The cell behavior and growth rate from one donor to another could slightly differ. Hence the duration of the osteogenic differentiation step and the number of medium exchanges between passages may vary from one donor to another.

1.4 Multi-Dimensional Induction of Cells

The multi-dimensional induction of ASCs was launched when cells reach a confluence and if a morphologic change appears and if at least one osteoid nodule (i.e., the un-mineralized, organic portion of the bone matrix that forms prior to the maturation of bone tissue) was observed in the flasks.

a) 3D-Induction in the Presence of Gelatin (NVD002 Biomaterial)

After being exposed to the osteogenic MD medium, the culture vessels containing the confluent monolayer of adherent osteogenic cells were slowly and homogeneously sprinkled with gelatin particles (Cultispher®-S, Percell Biolytica®, Astorp, Sweden) at a concentration of 1.5 cm³ for a 150 cm² vessel.

Cells were maintained in MD medium. Regular medium exchanges were performed every 3 to 4 days during the multi-dimensional induction. Those medium exchanges were performed by carefully preventing removal of gelatin particles and developing structure(s). The corresponding biomaterial is referred to as NVD002.

b) 3D-Induction in the Presence of HA/β-TCP (NVD003 Biomaterial)

After being exposed to the osteogenic MD medium, the culture vessels containing the confluent monolayer of adherent osteogenic cells were slowly and homogeneously sprinkled with HA/β-TCP particles (in a ratio 60/40): 3 cm³ for a 150 cm² flask (Biomatlante®, France).

Cells were maintained in MD medium. Regular medium exchanges were performed every 3 to 4 days during the multi-dimensional induction. Those medium exchanges were performed by carefully preventing removal of ceramic material particles and developing structure(s). The corresponding biomaterial is referred to as NVD003. The RNAs' content, in particular the miRNAs' content, was recovered from the obtained biomaterial, which constitutes a miRNAs cocktail.

1.5 miRNAs' Content

a) RNA Extraction

mRNAs isolation was performed from biopsies. mRNAs were extracted using miRNeasy kit Mastermix (Qiagen®, Hilden, Germany) following the manufacturer's protocol. RNA concentration was determined by Nanodrop (ThermoFisher®, Waltham, Mass., USA). To assess the quality of the samples, 2 μL of RNA was analyzed using RNA pico chip Agilent® Bioanalyzer (Agilent®, Santa Clara, Calif., USA). Three biological replicates were prepared per condition. Long RNA libraries were generated using TruSeq® Stranded Total RNA Library Prep (RS-122-2001, Illumina®, San Diego, Calif., USA) and small RNA libraries using the SMARTer® smRNA-Seq kit (635030, Takara Kusatsu, Shiga, Japan).

Sequencing was performed using an Illumina NextSeq® 500 (75-pb single-end reads) (Illumina®, San Diego, Calif., USA) generating approximately 26 million reads per long RNA libraries.

b) Ouantitative RT-PCR (qRT-PCR)

For quantification of miRNA expression, 50 ng RNA was reverse transcribed into cDNA using qScript miRNA cDNA Synthesis kit (Quanta Biosciences®), and qRT-PCR was conducted in triplicate using Perfecta SYBR Green Super Mix (Quanta Biosciences®). Thermal cycling was performed on an Applied Biosystems 7900 HT detection system (Applied Biosystems®). Data was normalized to miR-16-5p and U6 small nuclear RNA using the Delta-Delta Ct method.

c) Exosomes Purification

Exosomes have been isolated by differential centrifugation from culture medium whereby larger “contaminants” are first excluded by pelleting out through increasing speeds of centrifugation before exosomes, small extracellular vesicles and even protein aggregates are pelleted at very high speeds (˜100,000×g).

Briefly, the culture medium was collected and precleared by centrifugation at 400×g for 5 minutes, then 2,000×g for 20 min at 4° C., followed by centrifugation at 12,000×g for 45 min at 4° C. to eliminate dead cells and cellular debris. Then, the supernatant was passed through a 0.22-μm filter (Millipore®). The supernatant was then ultracentrifuged at 110,000×g for 120 minutes at 4° C., followed by washing of the exosome pellet with phosphate-buffered saline (PBS) at 110,000×g for 120 minutes at 4° C. (Optima XPN-80 Ultracentrifuge, Beckman Coulter®). The supernatant was discarded and the exosome pellet was lysed with Qiazol and stored at −80° C. for further analysis.

2. Results

a) Identification of miRNAs Obtained after 3D-Induction with Gelatin (NVD002)

TABLE 13
identification of miRNAs from purified exosomes

| | | | |
|---|---|---|---|
| hsa-let-7a-5p | hsa-miR-92a-3p | hsa-miR-92b-3p | hsa-miR-24-2-5p |
| hsa-let-7b-5p | hsa-miR-125b-5p | hsa-miR-335-5p | hsa-miR-26a-2-3p |
| hsa-let-7f-5p | hsa-miR-337-3p | hsa-let-7f-1-3p | hsa-miR-301a-3p |
| hsa-miR-24-3p | hsa-miR-93-5p | hsa-miR-196b-5p | hsa-miR-98-3p |
| hsa-miR-21-5p | hsa-miR-409-3p | hsa-miR-3613-3p | hsa-miR-1273a |
| hsa-miR-23b-3p | hsa-miR-199a-3p | hsa-miR-23a-5p | hsa-miR-28-5p |
| hsa-miR-1273g-3p | hsa-miR-145-5p | hsa-miR-374b-5p | hsa-miR-34a-3p |
| hsa-miR-574-3p | hsa-miR-30a-3p | hsa-miR-660-5p | hsa-miR-425-3p |
| hsa-miR-25-3p | hsa-miR-382-5p | hsa-miR-186-5p | hsa-miR-505-3p |
| hsa-let-7e-5p | hsa-miR-19b-3p | hsa-miR-454-3p | hsa-miR-34b-3p |
| hsa-miR-214-3p | hsa-miR-210-3p | hsa-miR-10a-5p | hsa-miR-361-3p |
| hsa-miR-199a-5p | hsa-miR-619-5p | hsa-miR-495-3p | hsa-miR-10b-5p |
| hsa-miR-196a-5p | hsa-miR-17-5p | hsa-miR-425-5p | hsa-miR-1306-5p |
| hsa-miR-199b-5p | hsa-miR-193a-5p | hsa-miR-2053 | hsa-miR-22-5p |
| hsa-miR-221-3p | hsa-miR-320b | hsa-miR-5096 | hsa-miR-378a-3p |
| hsa-miR-424-5p | hsa-miR-193b-5p | hsa-miR-494-3p | hsa-miR-411-5p |
| hsa-miR-23a-3p | hsa-miR-320a | hsa-miR-27a-3p | hsa-miR-505-5p |
| hsa-let-7c-5p | hsa-miR-151a-3p | hsa-miR-4449 | hsa-miR-664a-3p |
| hsa-miR-199b-3p | hsa-let-7a-3p | hsa-miR-532-3p | hsa-miR-26a-5p |
| hsa-miR-191-5p | hsa-miR-30e-3p | hsa-miR-532-5p | hsa-miR-377-3p |
| hsa-miR-574-5p | hsa-miR-22-3p | hsa-miR-126-5p | hsa-miR-485-3p |
| hsa-miR-424-3p | hsa-miR-99b-5p | hsa-miR-30c-5p | hsa-miR-590-3p |
| hsa-miR-423-5p | hsa-miR-625-3p | hsa-miR-130b-3p | hsa-miR-99a-3p |
| hsa-miR-342-3p | hsa-miR-4668-5p | hsa-miR-136-3p | hsa-miR-143-3p |
| hsa-let-7d-3p | hsa-miR-29b-3p | hsa-miR-15b-3p | hsa-miR-26b-3p |
| hsa-miR-130a-3p | hsa-miR-423-3p | hsa-miR-29b-1-5p | hsa-miR-3607-5p |
| hsa-miR-3184-3p | hsa-miR-376c-3p | hsa-miR-99b-3p | hsa-miR-3651 |
| hsa-miR-222-3p | hsa-let-7b-3p | hsa-miR-127-3p | hsa-miR-374a-3p |
| hsa-let-7g-5p | hsa-miR-3074-5p | hsa-miR-134-5p | hsa-miR-376a-3p |
| hsa-miR-125a-5p | hsa-miR-98-5p | hsa-miR-324-5p | hsa-miR-485-5p |
| hsa-let-7d-5p | hsa-miR-185-5p | hsa-miR-3605-3p | hsa-miR-103b |
| hsa-miR-29a-3p | hsa-miR-19a-3p | hsa-miR-101-3p | hsa-miR-126-3p |
| hsa-let-7i-5p | hsa-miR-34a-5p | hsa-miR-103a-3p | hsa-miR-149-5p |
| hsa-miR-146b-5p | hsa-miR-374c-3p | hsa-miR-1246 | hsa-miR-193b-3p |
| hsa-miR-4454 | hsa-miR-181a-5p | hsa-miR-138-5p | hsa-miR-223-3p |
| hsa-miR-28-3p | hsa-miR-328-3p | hsa-miR-190a-5p | hsa-miR-340-3p |
| hsa-miR-874-3p | hsa-miR-7847-3p | hsa-miR-6724-5p | hsa-miR-369-5p |

TABLE 14
identification of cellular miRNAs

| | | | |
|---|---|---|---|
| hsa-let-7a-5p | hsa-miR-210-3p | hsa-miR-29b-3p | hsa-miR-30e-3p |
| hsa-let-7b-5p | hsa-miR-3184-3p | hsa-miR-92a-3p | hsa-miR-320a |
| hsa-miR-24-3p | hsa-let-7d-5p | hsa-miR-193b-5p | hsa-miR-361-3p |
| hsa-miR-199a-5p | hsa-miR-25-3p | hsa-miR-181a-5p | hsa-miR-151a-3p |
| hsa-miR-214-3p | hsa-miR-193a-5p | hsa-miR-30c-5p | hsa-miR-154-5p |
| hsa-let-7f-5p | hsa-miR-199a-3p | hsa-miR-664b-3p | hsa-miR-664a-5p |
| hsa-miR-3607-5p | hsa-miR-29a-3p | hsa-miR-27a-3p | hsa-miR-92b-3p |
| hsa-miR-199b-3p | hsa-miR-342-3p | hsa-miR-320b | hsa-miR-1291 |
| hsa-let-7e-5p | hsa-miR-130a-3p | hsa-miR-3651 | hsa-miR-103b |
| hsa-miR-1273g-3p | hsa-miR-30a-3p | hsa-miR-664b-5p | hsa-miR-34a-3p |
| hsa-miR-125a-5p | hsa-miR-145-5p | hsa-miR-664a-3p | hsa-miR-140-5p |
| hsa-miR-21-5p | hsa-miR-28-3p | hsa-miR-98-5p | hsa-miR-3609 |
| hsa-let-7i-5p | hsa-miR-93-5p | hsa-miR-146b-5p | hsa-miR-374c-3p |
| hsa-miR-125b-5p | hsa-miR-34a-5p | hsa-miR-337-3p | hsa-miR-10a-5p |
| hsa-let-7g-5p | hsa-miR-222-3p | hsa-miR-4449 | hsa-miR-22-3p |
| hsa-miR-191-5p | hsa-miR-3074-5p | hsa-miR-6516-3p | hsa-miR-4668-5p |
| hsa-miR-574-3p | hsa-miR-424-5p | hsa-let-7i-3p | hsa-miR-24-2-5p |
| hsa-miR-199b-5p | hsa-miR-424-3p | hsa-miR-103a-3p | hsa-miR-29b-1-5p |
| hsa-miR-423-5p | hsa-miR-328-3p | hsa-miR-324-5p | hsa-miR-335-5p |
| hsa-miR-574-5p | hsa-miR-17-5p | hsa-miR-660-5p | hsa-miR-425-5p |
| hsa-miR-23b-3p | hsa-miR-23a-3p | hsa-miR-185-5p | hsa-miR-4461 |
| hsa-miR-196a-5p | hsa-let-7d-3p | hsa-miR-374b-5p | hsa-miR-127-3p |
| hsa-let-7c-5p | hsa-miR-423-3p | hsa-miR-409-3p | hsa-miR-196b-5p |
| hsa-miR-221-3p | hsa-miR-382-5p | hsa-miR-619-5p | hsa-miR-3613-5p |
| hsa-miR-3653-5p | hsa-miR-19b-3p | hsa-miR-99b-5p | hsa-miR-376c-3p |
| hsa-miR-99b-3p | hsa-miR-663b | hsa-miR-495-3p | hsa-miR-454-3p |

TABLE 15
Level of exosomal miRNAs from the NVD002
biomaterial as compared to a 2D culture

| miRNA | logFC | FC | logCPM | PValue | FDR |
|---|---|---|---|---|---|
| hsa-miR-3687 | −5.4340 | −43.2330 | 9.986598816 | 6.14E−08 | 3.38E−05 |
| hsa-miR-664b-5p | −6.7300 | −106.1558 | 9.571690269 | 1.19E−06 | 0.00032614 |
| hsa-miR-210-3p | 3.3558 | 10.2381 | 10.48338867 | 2.04E−06 | 0.00035487 |
| hsa-miR-4449 | −2.9142 | −7.5382 | 11.07025445 | 2.58E−06 | 0.00035487 |
| hsa-miR-3651 | −4.4165 | −21.3551 | 11.30176655 | 1.47E−05 | 0.00140376 |
| hsa-miR-663a | −5.5373 | −46.4407 | 10.73936527 | 1.53E−05 | 0.00140376 |
| hsa-miR-664b-3p | −7.2564 | −152.9025 | 10.0417161 | 2.26E−05 | 0.00177881 |
| hsa-miR-3653-5p | −3.7408 | −13.3696 | 9.73359759 | 5.05E−05 | 0.00347481 |
| hsa-miR-664a-3p | −2.8640 | −7.2806 | 10.34200935 | 0.000189789 | 0.01159819 |
| hsa-miR-3648 | −3.5109 | −11.4000 | 9.789761883 | 0.000273373 | 0.01503553 |
| hsa-miR-619-5p | 1.9219 | 3.7892 | 10.62269524 | 0.002222957 | 0.11114785 |
| hsa-miR-181a-5p | 2.5395 | 5.8140 | 9.387142262 | 0.002792503 | 0.12798972 |
| hsa-miR-409-3p | −1.3868 | −2.6151 | 12.81792844 | 0.00335429 | 0.14191227 |
| hsa-let-7a-3p | 6.5703 | 95.0297 | 9.606951776 | 0.004329701 | 0.17009541 |
| hsa-miR-4454 | 2.6272 | 6.1784 | 11.53331453 | 0.004846818 | 0.17771667 |
| hsa-let-7i-5p | 1.4660 | 2.7626 | 11.86103511 | 0.006780515 | 0.23308021 |
| hsa-miR-335-5p | 3.5260 | 11.5196 | 9.315665201 | 0.007991541 | 0.25854987 |
| hsa-miR-1246 | 5.0336 | 32.7554 | 8.539445396 | 0.009781646 | 0.29888364 |
| hsa-miR-6516-5p | −4.1176 | −17.3593 | 9.073414111 | 0.011915311 | 0.34491691 |
| hsa-miR-3607-5p | −2.0844 | −4.2410 | 9.626131484 | 0.012915156 | 0.35516678 |
| hsa-let-7e-5p | 1.3793 | 2.6014 | 13.88843995 | 0.016482404 | 0.43168201 |
| hsa-miR-25-3p | −1.0699 | −2.0993 | 14.99237678 | 0.020116155 | 0.50290388 |
| hsa-miR-374c-3p | 2.1608 | 4.4719 | 9.494278145 | 0.0222919 | 0.53306717 |
| hsa-miR-29b-3p | 1.5768 | 2.9832 | 9.845249887 | 0.025174313 | 0.56328252 |
| hsa-let-7b-3p | −1.4233 | −2.6820 | 10.95150956 | 0.025603751 | 0.56328252 |
| hsa-miR-23b-3p | 1.2910 | 2.4470 | 14.73760912 | 0.028045493 | 0.5796539 |
| hsa-miR-3613-3p | −2.0169 | −4.0472 | 10.92938585 | 0.028455737 | 0.5796539 |
| hsa-miR-138-5p | −1.5674 | −2.9637 | 9.777277597 | 0.040019769 | 0.78610261 |
| hsa-miR-6516-3p | −2.0847 | −4.2420 | 8.826423155 | 0.042870909 | 0.81306896 |

TABLE 16
Level of cellular miRNAs from the NVD002
biomaterial as compared to a 2D culture

| miRNA | logFC | FC | logCPM | PValue | FDR |
|---|---|---|---|---|---|
| hsa-miR-210-3p | 6.4208 | 85.6764 | 12.48420101 | 6.80E−18 | 2.42E−15 |
| hsa-miR-4485-3p | −4.0409 | −16.4603 | 11.3547262 | 8.18E−11 | 1.46E−08 |
| hsa-miR-24-3p | 1.1078 | 2.1551 | 16.03988644 | 2.58E−05 | 0.0030666 |
| hsa-miR-409-3p | −2.2145 | −4.6413 | 11.39958036 | 0.000257967 | 0.02295903 |
| hsa-let-7i-5p | 1.1353 | 2.1966 | 13.67239011 | 0.000499245 | 0.03554627 |
| hsa-miR-382-5p | −1.2517 | −2.3813 | 11.92251263 | 0.00225027 | 0.133516 |
| hsa-miR-214-3p | 0.8790 | 1.8391 | 15.62352032 | 0.003282859 | 0.16695684 |
| hsa-miR-199b-5p | 1.2889 | 2.4435 | 13.44753159 | 0.006034794 | 0.24998011 |
| hsa-miR-199a-5p | 0.8790 | 1.8391 | 15.68100144 | 0.006319722 | 0.24998011 |
| hsa-miR-3074-5p | 1.2659 | 2.4048 | 11.37814998 | 0.009651574 | 0.34359603 |
| hsa-miR-361-3p | 2.8526 | 7.2232 | 9.80277522 | 0.013351705 | 0.43210973 |
| hsa-miR-6723-5p | −4.6621 | −25.3195 | 9.311990262 | 0.015695848 | 0.46564349 |
| hsa-miR-130a-3p | 1.7314 | 3.3205 | 12.0290808 | 0.017837723 | 0.48847918 |
| hsa-miR-663a | −2.7768 | −6.8533 | 9.692314667 | 0.019868045 | 0.505216 |
| hsa-miR-3607-5p | 0.6038 | 1.5197 | 14.71277969 | 0.021552995 | 0.5115244 |
| hsa-miR-660-5p | 1.5747 | 2.9787 | 10.15389819 | 0.025273272 | 0.5623303 |
| hsa-miR-196b-5p | −1.6406 | −3.1181 | 10.12403868 | 0.028246436 | 0.5915136 |
| hsa-miR-4449 | −1.1855 | −2.2744 | 11.22700361 | 0.031038083 | 0.61386431 |
| hsa-miR-342-3p | 0.9434 | 1.9230 | 12.22137602 | 0.038570501 | 0.70780213 |
| hsa-miR-221-3p | −0.7419 | −1.6723 | 13.67334394 | 0.039764165 | 0.70780213 |

b) Identification of miRNAs Obtained after 3D-Induction with HA/β-TCP (NVD003)

TABLE 17
identification of miRNAs from purified exosomes

| | | | |
|---|---|---|---|
| hsa-let-7a-5p | hsa-let-7i-5p | hsa-miR-660-5p | hsa-miR-6832-3p |
| hsa-let-7b-5p | hsa-miR-409-3p | hsa-miR-664a-3p | hsa-miR-146a-5p |
| hsa-miR-24-3p | hsa-miR-210-3p | hsa-miR-185-5p | hsa-miR-16-2-3p |
| hsa-miR-21-5p | hsa-miR-199a-3p | hsa-miR-3651 | hsa-miR-181b-5p |
| hsa-let-7f-5p | hsa-miR-30a-3p | hsa-miR-495-3p | hsa-miR-26a-2-3p |
| hsa-miR-574-3p | hsa-miR-320b | hsa-let-7a-3p | hsa-miR-376a-3p |
| hsa-miR-23b-3p | hsa-miR-193a-5p | hsa-miR-28-5p | hsa-miR-539-5p |
| hsa-miR-1273g-3p | hsa-miR-382-5p | hsa-miR-99b-3p | hsa-miR-708-5p |
| hsa-miR-25-3p | hsa-miR-423-3p | hsa-miR-103a-3p | hsa-miR-98-3p |
| hsa-miR-199a-5p | hsa-miR-17-5p | hsa-miR-19a-3p | hsa-miR-1237-5p |
| hsa-miR-196a-5p | hsa-miR-19b-3p | hsa-miR-126-5p | hsa-miR-223-3p |
| hsa-miR-214-3p | hsa-miR-92b-3p | hsa-miR-2053 | hsa-miR-532-5p |
| hsa-miR-125a-5p | hsa-miR-320a | hsa-miR-29b-1-5p | hsa-miR-542-3p |
| hsa-miR-221-3p | hsa-miR-3074-5p | hsa-miR-3648 | hsa-miR-663a |
| hsa-miR-222-3p | hsa-miR-376c-3p | hsa-miR-374a-3p | hsa-miR-101-3p |
| hsa-let-7e-5p | hsa-let-7b-3p | hsa-miR-454-3p | hsa-miR-143-3p |
| hsa-miR-191-5p | hsa-miR-625-3p | hsa-miR-532-3p | hsa-miR-21-3p |
| hsa-miR-199b-3p | hsa-miR-99b-5p | hsa-miR-136-3p | hsa-miR-224-5p |
| hsa-miR-342-3p | hsa-miR-34a-5p | hsa-miR-361-3p | hsa-miR-26a-5p |
| hsa-miR-23a-3p | hsa-miR-5096 | hsa-miR-1246 | hsa-miR-27a-5p |
| hsa-miR-424-3p | hsa-miR-30e-3p | hsa-miR-130b-3p | hsa-miR-324-5p |
| hsa-miR-28-3p | hsa-miR-22-3p | hsa-miR-134-5p | hsa-miR-340-3p |
| hsa-let-7g-5p | hsa-miR-151a-3p | hsa-miR-154-5p | hsa-miR-379-5p |
| hsa-miR-92a-3p | hsa-miR-186-5p | hsa-miR-34a-3p | hsa-miR-409-5p |
| hsa-miR-424-5p | hsa-miR-193b-5p | hsa-miR-576-5p | hsa-miR-543 |
| hsa-let-7d-3p | hsa-miR-328-3p | hsa-miR-874-3p | hsa-miR-5787 |
| hsa-miR-4454 | hsa-miR-4449 | hsa-miR-100-5p | hsa-miR-6089 |
| hsa-miR-146b-5p | hsa-miR-27a-3p | hsa-miR-103b | hsa-miR-127-3p |
| hsa-miR-423-5p | hsa-miR-30c-5p | hsa-miR-1273a | hsa-miR-149-5p |
| hsa-miR-29a-3p | hsa-miR-494-3p | hsa-miR-1306-5p | hsa-miR-181c-5p |
| hsa-miR-574-5p | hsa-miR-98-5p | hsa-miR-138-5p | hsa-miR-193b-3p |
| hsa-miR-199b-5p | hsa-miR-10a-5p | hsa-miR-15b-3p | hsa-miR-222-5p |
| hsa-miR-125b-5p | hsa-miR-29b-3p | hsa-miR-26b-3p | hsa-miR-3613-5p |
| hsa-miR-3184-3p | hsa-miR-374b-5p | hsa-miR-10b-5p | hsa-miR-365b-3p |
| hsa-let-7c-5p | hsa-miR-335-5p | hsa-miR-22-5p | hsa-miR-3960 |
| hsa-miR-33 7-3p | hsa-miR-374c-3p | hsa-miR-3613-3p | hsa-miR-485-3p |
| hsa-let-7d-5p | hsa-miR-425-5p | hsa-miR-655-3p | hsa-miR-6087 |
| hsa-miR-145-5p | hsa-miR-181a-5p | hsa-miR-7-1-3p | hsa-miR-92a-1-5p |
| hsa-miR-93-5p | hsa-miR-196b-5p | hsa-miR-23a-5p | hsa-miR-4668-5p |
| hsa-miR-619-5p | hsa-let-7f-1-3p | hsa-miR-24-2-5p | hsa-miR-3605-3p |
| hsa-miR-130a-3p | | | |

TABLE 18
identification of cellular miRNAs

| | | | |
|---|---|---|---|
| hsa-let-7a-5p | hsa-miR-3653-5p | hsa-miR-98-5p | hsa-miR-28-5p |
| hsa-let-7b-5p | hsa-miR-342-3p | hsa-miR-664a-3p | hsa-miR-10a-5p |
| hsa-miR-24-3p | hsa-miR-28-3p | hsa-miR-92b-3p | hsa-miR-151a-3p |
| hsa-let-7f-5p | hsa-miR-23b-3p | hsa-miR-4449 | hsa-miR-30e-3p |
| hsa-miR-199a-5p | hsa-let-7c-5p | hsa-miR-320a | hsa-miR-324-5p |
| hsa-miR-214-3p | hsa-miR-222-3p | hsa-miR-181a-5p | hsa-miR-495-3p |
| hsa-miR-3607-5p | hsa-miR-29a-3p | hsa-miR-3651 | hsa-miR-576-5p |
| hsa-miR-125a-5p | hsa-miR-92a-3p | hsa-miR-185-5p | hsa-miR-625-3p |
| hsa-miR-199b-3p | hsa-miR-30a-3p | hsa-miR-664b-5p | hsa-miR-671-5p |
| hsa-miR-125b-5p | hsa-miR-424-3p | hsa-miR-196b-5p | hsa-miR-1271-5p |
| hsa-miR-21-5p | hsa-miR-423-3p | hsa-miR-27a-3p | hsa-miR-186-5p |
| hsa-let-7e-5p | hsa-miR-34a-5p | hsa-miR-29b-3p | hsa-miR-23a-5p |
| hsa-let-7i-5p | hsa-miR-424-5p | hsa-miR-664b-3p | hsa-miR-3613-5p |
| hsa-let-7g-5p | hsa-miR-145-5p | hsa-miR-99b-5p | hsa-miR-376c-3p |
| hsa-miR-574-3p | hsa-miR-328-3p | hsa-miR-103a-3p | hsa-miR-409-3p |
| hsa-miR-574-5p | hsa-miR-3074-5p | hsa-miR-6516-3p | hsa-miR-4461 |
| hsa-miR-191-5p | hsa-let-7d-3p | hsa-miR-22-3p | hsa-miR-454-3p |
| hsa-miR-196a-5p | hsa-miR-93-5p | hsa-miR-26a-5p | hsa-miR-6724-5p |
| hsa-miR-221-3p | hsa-miR-23a-3p | hsa-miR-103b | hsa-let-7b-3p |
| hsa-miR-25-3p | hsa-miR-19b-3p | hsa-miR-1291 | hsa-miR-190a-5p |
| hsa-miR-423-5p | hsa-miR-146b-5p | hsa-miR-425-5p | hsa-miR-26b-3p |
| hsa-miR-210-3p | hsa-miR-320b | hsa-miR-22-5p | hsa-miR-3609 |
| hsa-miR-1273g-3p | hsa-miR-337-3p | hsa-miR-374c-3p | hsa-miR-411-5p |
| hsa-let-7d-5p | hsa-miR-17-5p | hsa-let-7i-3p | hsa-miR-425-3p |
| hsa-miR-199b-5p | hsa-miR-130a-3p | hsa-miR-374b-5p | hsa-miR-4485-3p |
| hsa-miR-199a-3p | hsa-miR-193b-5p | hsa-miR-455-3p | hsa-miR-30c-5p |
| hsa-miR-193a-5p | hsa-miR-382-5p | hsa-miR-532-3p | hsa-miR-619-5p |
| hsa-miR-3184-3p | | | |

TABLE 19
Level of exosomal miRNAs from the NVD003
biomaterial as compared to a 2D culture

| miRNA | logFC | FC | logCPM | PValue | FDR |
|---|---|---|---|---|---|
| hsa-miR-210-3p | 3.8329 | 14.2502 | 11.0073 | 5.86E−07 | 0.00016341 |
| hsa-miR-3687 | −4.2961 | −19.6462 | 10.1302 | 4.12E−07 | 0.00016341 |
| hsa-miR-4454 | 3.3561 | 10.2401 | 12.1672 | 1.14E−06 | 0.00021296 |
| hsa-miR-3653-5p | −4.4413 | −21.7264 | 9.7928 | 3.76E−06 | 0.00052506 |
| hsa-miR-664b-5p | −4.2939 | −19.6152 | 9.7186 | 9.08E−06 | 0.00101337 |
| hsa-miR-619-5p | 2.7545 | 6.7485 | 11.3607 | 0.00016339 | 0.0130247 |
| hsa-miR-664b-3p | −4.9289 | −30.4622 | 10.1914 | 0.00014999 | 0.0130247 |
| hsa-miR-3613-3p | −3.0938 | −8.5377 | 10.8210 | 0.00105987 | 0.07392561 |
| hsa-miR-409-3p | −1.5541 | −2.9365 | 12.8917 | 0.00139377 | 0.08641401 |
| hsa-miR-3607-5p | −2.9445 | −7.6981 | 9.6272 | 0.00178514 | 0.0996108 |
| hsa-miR-1246 | 5.1208 | 34.7953 | 8.6663 | 0.00292854 | 0.14855699 |
| hsa-miR-3609 | −4.9838 | −31.6427 | 8.4767 | 0.00415237 | 0.19308534 |
| hsa-miR-222-3p | 1.4242 | 2.6837 | 13.2032 | 0.0053805 | 0.23094758 |
| hsa-miR-181a-5p | 2.3137 | 4.9717 | 9.3588 | 0.00612351 | 0.24406574 |
| hsa-miR-6832-3p | 4.7957 | 27.7766 | 8.5024 | 0.01014361 | 0.37734231 |
| hsa-miR-335-5p | 3.3247 | 10.0198 | 9.3569 | 0.01119602 | 0.39046125 |
| hsa-let-7a-3p | 5.8907 | 59.3317 | 9.0729 | 0.01316075 | 0.40798328 |
| hsa-miR-28-3p | 1.2784 | 2.4258 | 12.7300 | 0.01298217 | 0.40798328 |
| hsa-miR-663a | −3.4819 | −11.1732 | 10.9716 | 0.01441164 | 0.42324701 |
| hsa-miR-19a-3p | −1.6552 | −3.1497 | 10.3578 | 0.01740016 | 0.46234701 |
| hsa-miR-3651 | −2.7857 | −6.8957 | 11.5575 | 0.01725774 | 0.46234701 |
| hsa-miR-125a-5p | 1.2160 | 2.3231 | 13.5469 | 0.0201696 | 0.51157428 |
| hsa-miR-374c-3p | 2.1030 | 4.2963 | 9.5512 | 0.02120255 | 0.51439235 |
| hsa-miR-4668-5p | −1.6714 | −3.1853 | 10.65127 | 0.02283107 | 0.53082232 |
| hsa-miR-301a-3p | −2.8147 | −7.0361 | 8.9635 | 0.02430933 | 0.54258424 |
| hsa-miR-664a-3p | −1.7910 | −3.4606 | 10.6389 | 0.02619156 | 0.56211116 |
| hsa-miR-485-5p | −2.0114 | −4.0317 | 9.0528 | 0.03141568 | 0.64925748 |
| hsa-miR-382-5p | −1.0946 | −2.1355 | 12.0209 | 0.03304208 | 0.65848152 |
| hsa-miR-4449 | −1.5489 | −2.9260 | 11.4255 | 0.03601783 | 0.68391665 |
| hsa-miR-138-5p | −1.5886 | −3.0076 | 9.8986 | 0.03676971 | 0.68391665 |
| hsa-miR-181c-5p | 4.1112 | 17.2823 | 8.2063 | 0.04528724 | 0.81517026 |
| hsa-miR-374b-5p | 1.7817 | 3.4383 | 9.6939 | 0.04793018 | 0.83578254 |

TABLE 20
Level of cellular miRNAs from the NVD003
biomaterial as compared to a 2D culture

| miRNA | logFC | FC | logCPM | PValue | FDR |
|---|---|---|---|---|---|
| hsa-miR-210-3p | 6.4907 | 89.9288 | 12.5959 | 3.65E−17 | 1.29E−14 |
| hsa-miR-4485-3p | −3.7281 | −13.2516 | 11.4563 | 3.29E−09 | 5.82E−07 |
| hsa-miR-409-3p | −3.3163 | −9.9616 | 11.31065 | 3.00E−06 | 0.00035375 |
| hsa-miR-382-5p | −1.6105 | −3.05363 | 11.8932 | 0.00020693 | 0.01831299 |
| hsa-miR-24-3p | 0.9484 | 1.9298 | 16.0201 | 0.00036741 | 0.0260129 |
| hsa-miR-93-5p | −1.5223 | −2.8725 | 12.3200 | 0.00054198 | 0.03197659 |
| hsa-let-7i-5p | 1.1389 | 2.2021 | 13.7506 | 0.00119986 | 0.06067875 |
| hsa-miR-3074-5p | 1.2618 | 2.3980 | 11.4482 | 0.00741164 | 0.32796499 |
| hsa-let-7c-5p | −0.9293 | −1.9044 | 13.0382 | 0.01316331 | 0.47049582 |
| hsa-miR-154-5p | −2.0724 | −4.2061 | 10.1415 | 0.01329084 | 0.47049582 |
| hsa-miR-6723-5p | −4.7630 | −27.1540 | 9.3165 | 0.01570223 | 0.50532623 |
| hsa-miR-671-5p | 4.2840 | 19.4815 | 9.3994 | 0.03498742 | 1 |

### Example 2

### Skin Wound Reconstruction Properties of NVD002-Derived Exosomes

The potential functional impact of NVD002-derived exosomes was studied in vitro on one model of cell line: HDFa (human dermal fibroblast).

NVD002-derived exosomes (ASCs osteo-differentiated and 3D-induced in the presence of gelatin, as in example 1) from 3 donors were co-incubated in 96-wells plates with HDFa cell line at 2.5 and 25 μg/ml for up to 72 h at 37° C., 5% CO₂ under normoxia (21% O₂) or hypoxia (1% O₂). A cell viability test (CellTiter-Glo Cell viability Assay) was performed after 30 minutes to 48 h of co-incubation, at minimum 5 different time points, to evaluate the proliferation of HDFa. The CellTiter-Gloe Luminescent Cell Viability Assay is a homogeneous method to determine the number of viable cells in culture based on quantitation of the ATP present, which signals the presence of metabolically active cells. Experiments were performed in triplicate.

Statistically significant differences between groups (with normal distribution) were tested by paired t-test and two-way analysis of variance with the Bonferroni post hoc test. Non-normal distributions of data were analyzed using the Kruskal-Wallis test. Statistical tests were performed with Prism GraphPad 2 (NIH). A P value<0.05 was considered significant.

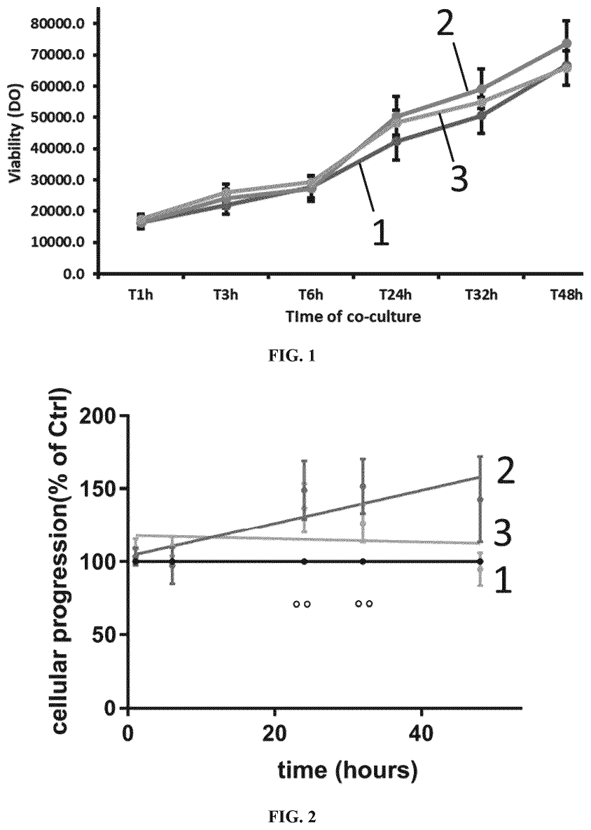

Proliferation curves of HDFa cells cultured with NVD002-derived exosomes under normoxia (curves 2 and 3 of FIG. 1) showed a slightly higher level of viability than the control cells cultured without exosomes (curve 1). In addition, a more marked effect was noted with the lowest dose of exosomes (2.5 μg/ml vs 25 μg/ml) (FIG. 1).

Linear regression of the progression speed curves was calculated. Higher progression speed rates were found for HDFa cells cultured with exosomes, at both 2.5 and 25 μg/ml. A significant higher viability was observed for HDFa cells co-cultured with NVD002-derived exosomes at 2.5 and 2.5 μg/ml at 24 h and 32 h/48 h of incubation of incubation (p<0.01). (FIG. 2).

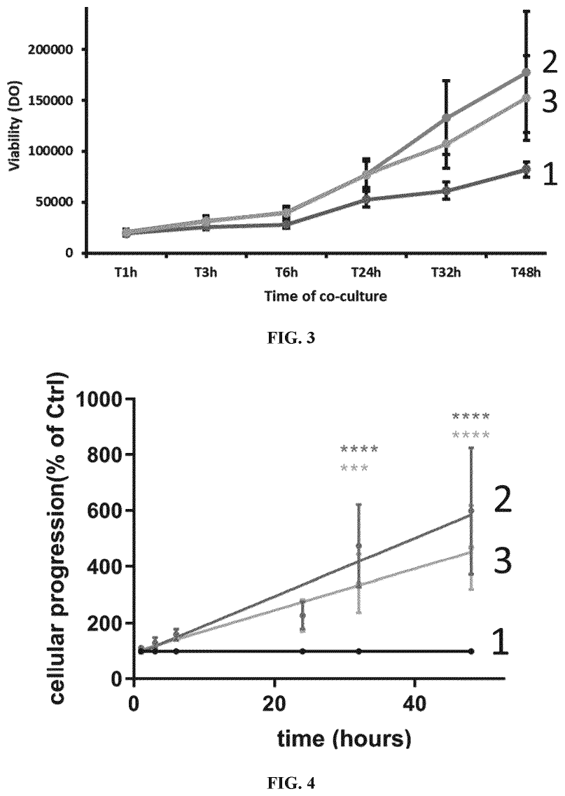

Proliferation curves of HDFa cells cultured with NVD002-derived exosomes under hypoxia (curves 2 and 3 of FIG. 3) showed a higher level of viability than the control cells cultured without exosomes (curve 1). In addition, a more marked effect was noted with the lowest dose of exosomes (2.5 μg/ml vs 25 μg/ml) (FIG. 3).

Linear regression of the progression speed curves was calculated. higher proliferation rates were found for HDFa cells cultured with exosomes, at both 2.5 and 25 μg/ml. A significant higher viability was observed for HDFa cells co-cultured with NVD002-derived exosomes at 2.5 and 2.5 μg/ml at 24 h and 32 h/48 h of incubation of incubation (p<0.01). (FIG. 4).

In conclusion, NVD002-derived exosomes can speed the proliferation of human dermal fibroblast cell lines in vitro. These results suggest that skin repair, including, e.g., the diabetic wound healing, may be achieved with NVD002-derived exosomes.

### Example 3

### In Vitro Evaluation of the Inhibition of Osteoclastogenesis

1. Impact of NVD003-Derived Exosomes (miRNAs Cocktail) on the Inhibition of Osteoclasts Maturation and Activity

1.1 Impact of the NVD003-Derived Exosomes on the Differentiation of Osteoclasts Precursors

a) Osteoclastogenesis in Presence of RANKL

An in vitro osteoclast differentiation protocol from human monocytes was used. Human CD14+ monocytes were isolated from peripheral blood of healthy volunteers, obtained in agreement with the “Etablissement Francais du Sang”.

Following isolation of peripheral blood mononuclear cells by Ficoll-Hypaque centrifugation, monocytes (CD14+ cells) were sorted (MACS®, MiltenyiBiotec). Freshly isolated precursors were differentiated into osteoclasts in the presence of M-CSF and RANKL (RANKL medium, “plus RANKL” control). Cells in medium without RANKL serve as a negative control (M-CSF medium, “no RANKL” control). The differentiation time was 8 days. The osteoclatogenesis was carried out in 96-well culture plates.

At D0, precursors (CD14+) were seeded and incubated for 2 hours (minimum time for cell attachment) in medium supplemented with 1% FBS, 25 ng/mL human MCSF+/−100 ng/mL human RANKL and exosomes were added at 50 and 100 μg/ml in 24-well culture plates. Medium was changed at day 4 and day 7. All treatments and controls were carried out in triplicate.

A TRAP staining was performed at day 8. The number of TRAP-positive cells containing more than three nuclei was determined in each well.

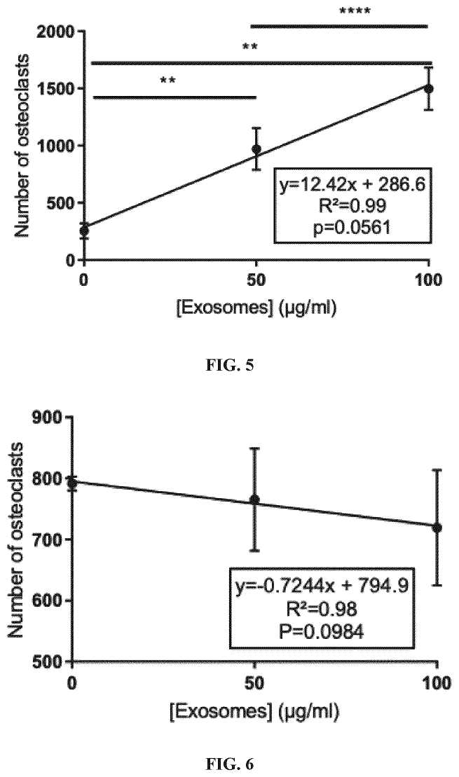

As shown in FIG. 5, a strong dose-dependent increase of the mean osteoclast number was observed in the presence of exosomes. The mean percent of increase induced by exosomes from the 3 donors is 280.70±31.10 at 50 μg/mL and 486.93±18.44 at 100 μg/mL.

b) Osteoclastogenesis in Absence of RANKL+/−Sclerostin

Human CD14+ monocytes were isolated from peripheral blood of healthy volunteers, obtained in agreement with the “Etablissement Français du Sang”. Following isolation of peripheral blood mononuclear cells by Ficoll-Hypaque centrifugation, monocytes (CD14+ cells) were sorted (MACS®, Miltenyi Biotec). Freshly isolated precursors (CD14+) were cultivated in M-CSF medium (medium supplemented with 1% FBS and 25 ng/mL M-CSF) with or without exosomes and/or sclerostin. Cells in RANKL medium (medium supplemented with 1% FBS, 25 ng/mL M-CSF and 100 ng/mL RANKL) served as positive control. The osteoclatogenesis was carried out in 96-well cell culture plates. At D0, precursors (CD14+) were seeded and incubated for 2 hours (minimum time for cell attachment) in 50 μL of medium supplemented with 1 FBS, 25 ng/mL human M-CSF+/−100 ng/mL human RANKL. Then, exosomes and/or sclerostin were added.

All treatments and controls were carried out in duplicate. Media (and treatments) were changed at day 4 and day 7. A TRAP staining was performed at day 8. The number of TRAP-positive cells containing more than three nuclei was determined in each well.

In the absence of RANKL, no osteoclast was formed. No osteoclast was formed in the presence of sclerostin at 10 ng/mL or 100 ng/mL. The combination of exosomes and sclerostin was not more able to induce osteoclast formation in the absence of RANKL.

1.2 Impact of the NVD003-Derived Exosomes on the Mature Osteoclasts (Cytotoxicity)

Human CD14+ monocytes were isolated from peripheral blood of healthy volunteers, obtained in agreement with the “Etablissement Français du Sang”. Osteoclast precursor cells were isolated from the peripheral blood. Following isolation of peripheral blood mononuclear cells by Ficoll-Hypaque centrifugation, monocytes (CD14+ cells) were sorted (MACS®, Miltenyi Biotec). Freshly isolated precursors were differentiated into osteoclasts in the presence of M-CSF and RANKL (“plus RANKL” control) for 5 to 6 days (depending on the donor of CD14+ cells). Cells in medium without RANKL served as a negative control (“no RANKL” control). The differentiation time was 8 days. The osteoclatogenesis was carried out in 96-well culture plates.

At D0, precursors (CD14+) were seeded and incubated for 2 hours (minimum time for cell attachment) in 50 μL of medium supplemented with 1 FBS, 25 ng/mL human M-CSF+/−100 ng/mL human RANKL.

When the multinucleated cells were observed in the positive control, medium was renewed. All treatments and controls were carried out in triplicate.

48 hours after the addition of the NVD003-derived exosomes, a TRAP staining was performed. The number of TRAP-positive cells containing more than three nuclei was determined in each well.

As shown in FIG. 6, the mean osteoclast number per well was not significantly modified by treatment with exosomes. The treatment with exosomes showed no effect at the two tested concentrations of 50 μg/mL and 100 μg/mL (% of inhibition of 3.34% and 9.15%, respectively).

2. Impact of NVD003-Derived Exosomes (miRNA Cocktail) on the Promotion of Osteogenesis

2.1 Impact of NVD003-Derived Exosomes on ASCs Osteodifferentiation

Adipose tissue-derived stem cells (ASCs) at passage 5 (P5) were placed in 96 wells plates in 0.1 mL of proliferation medium (MP) (in the presence of 5% hPL) for about 2 days. Then, the MP was removed and the cells were rinsed 2-fold with PBS. Cells were placed in proliferation medium (MP) or in osteo-differentiation medium (MD), MD+sclerostin (SCL) 100 ng/ml or MD+sclerostin (SCL) 100 ng/ml+NVD003-derived exosomes 100 μg/ml, for 10 days with medium change after 5 days. In addition, cells were placed in proliferation medium (MP) as negative control.

After 10 days of culture, cells were placed in Qiazol lysis reagent (Qiagen®, Hilden, Germany) for RNA isolation for qRT-PCR for osteogenic genes. Total RNA was extracted from cell lysates. RNAs were purified using Rneasy mini kit (Qiagen®, Hilden, Germany) with an additional on column DNase digestion according to the manufacturer's instruction. Quality and quantity of RNA were determined using a spectrophotometer (Spectramax 190, Molecular Devices®, California, USA). cDNA was synthesized from 0.5 μg of total RNA using RT² RNA first strand kit (Qiagene, Hilden, Germany) for osteogenic and angiogenic genes expression profiles though customized PCR arrays (Customized Human Osteogenic and angiogenic RT² Profiler Assay—Qiagen®, Hilden, Germany). The ABI Quantstudio 5 system (Applied Biosystemse) and SYBR Green ROX Mastermix (Qiagen®, Hilden, Germany) were used for detection of the amplification product. Quantification was obtained according to the ΔΔCT method. The final result of each sample was normalized to the means of expression level of three housekeeping genes (ACTB, B2M and GAPDH). Experiments were performed in triplicate.

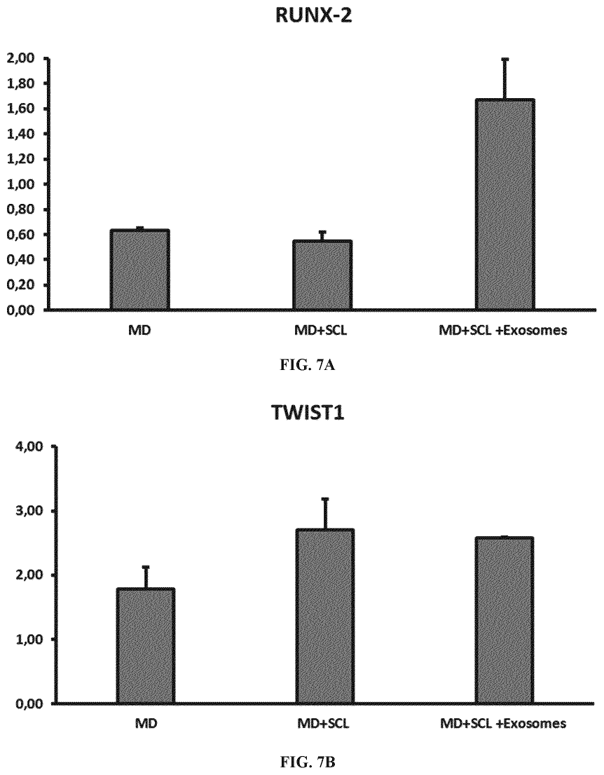

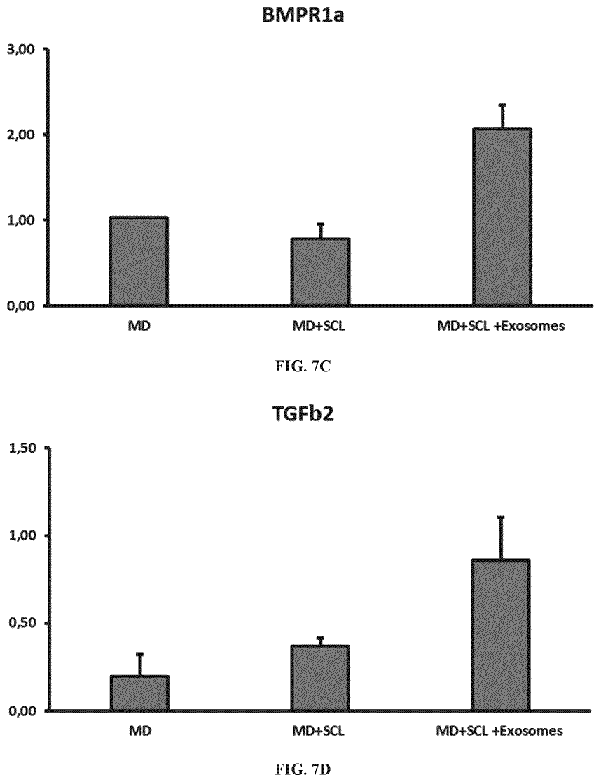

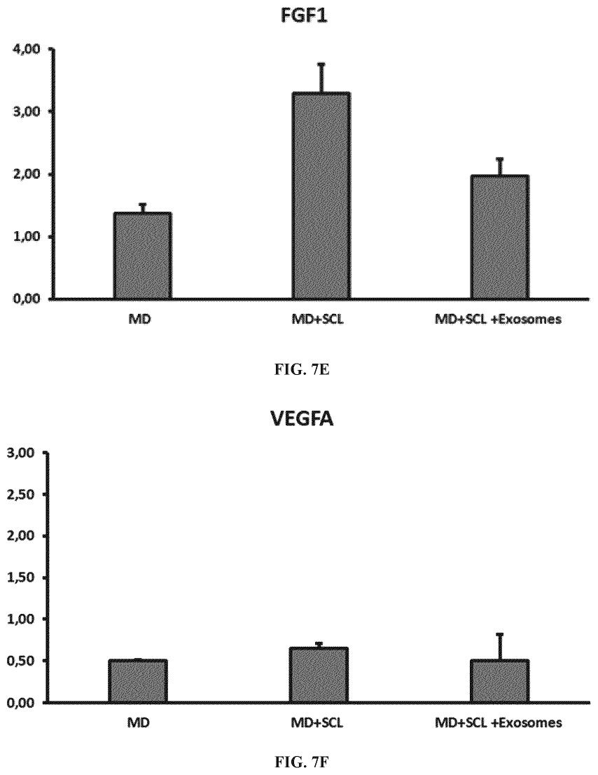

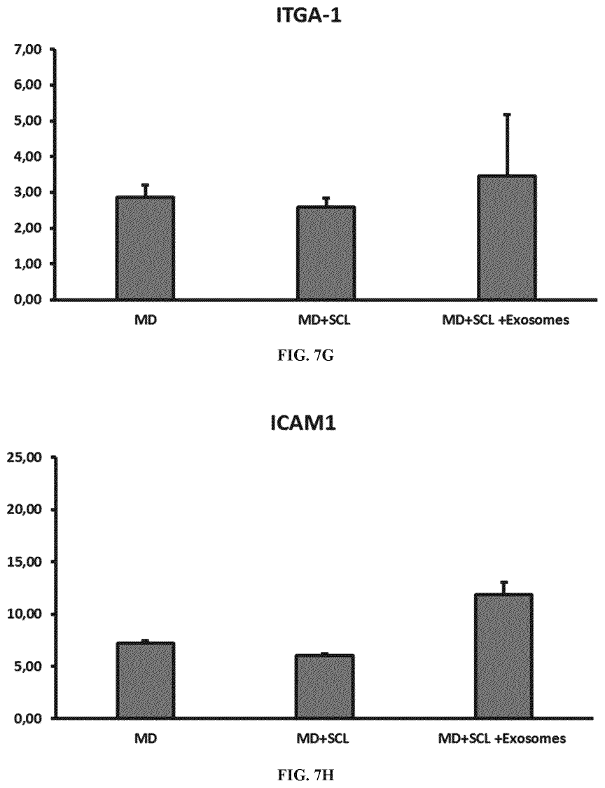

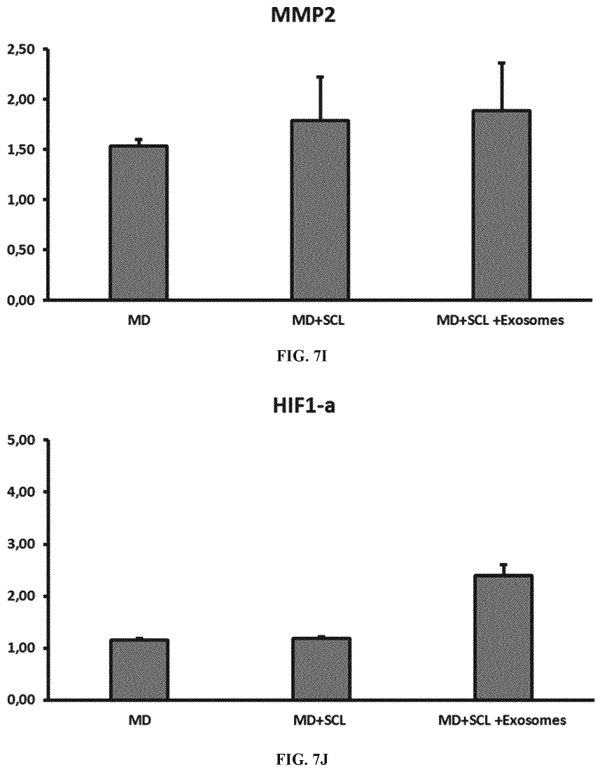

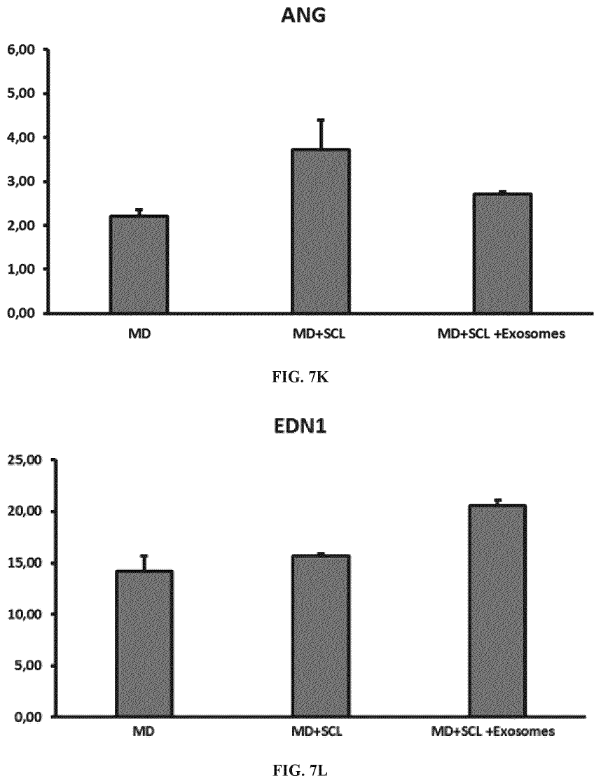

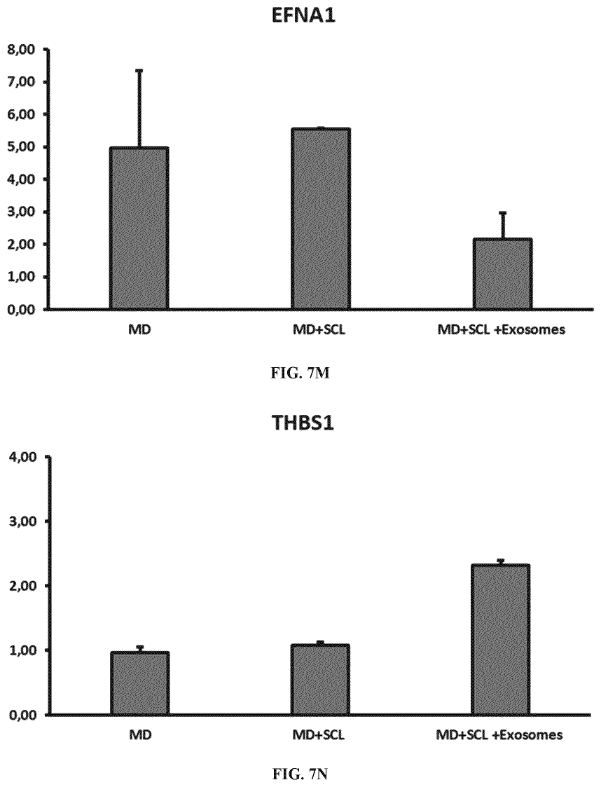

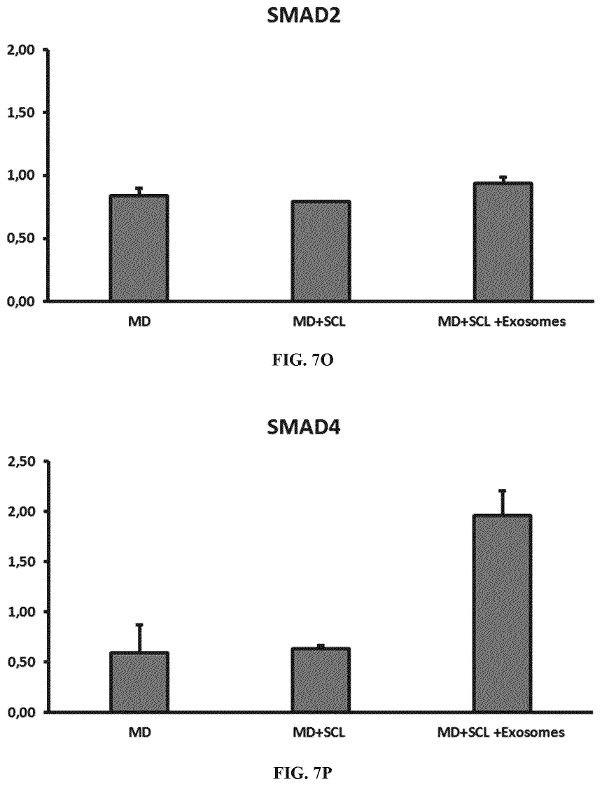

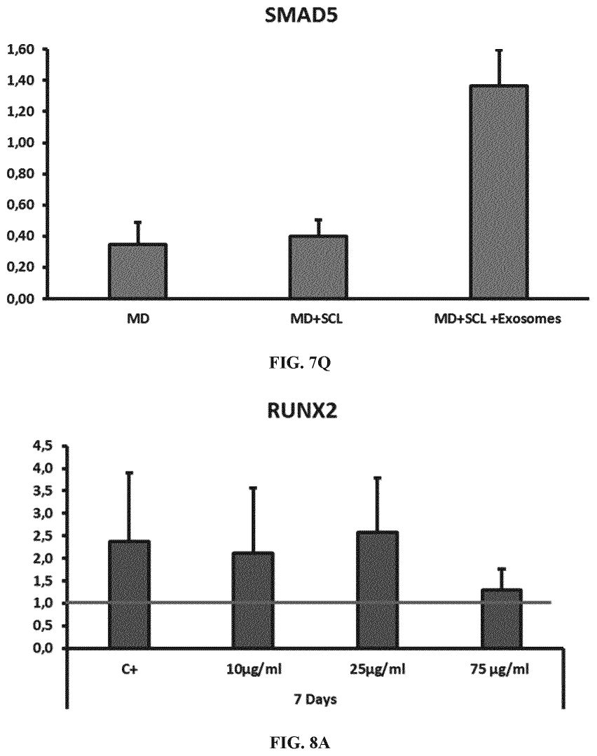

FIG. 7A-Q shows the osteogenic genes expression by ASCs placed in osteo-differentiation medium (MD), MD+100 ng/ml SCL and MD+100 ng/ml SCL+100 μg/ml NVD003-derived exosomes. Results are shown as mean+/−SD, as a fold induction compared to proliferation medium.

Surprisingly, culture of ASCs in MD did not induce the expression of all tested osteogenic genes. An overexpression of osteogenic and angiogenic genes MMP, ITGA1, HIF1a, FGF1, ANG, EDN1, TWIST1 and ICAM1 was found in comparison with ASCs in proliferation medium. No impact of SCL was noted on osteogenic genes expression. In contrast, the coculture of exosomes showed the overexpression of skeletal development factors, such as RUNX2 (FIG. 7A), TWIST1 (FIG. 7B), BMPR1A (FIG. 7C); of growth factor FGF1 (FIG. 7E); of cell-extracellular matrix adhesion factors, such as, ITGA1 (FIG. 7G) and ICAM1 (FIG. 7H); of angiogenic factors, such as, MMP2 (FIG. 7I), HIF1a (FIG. 7J), ANG (FIG. 7K), EDN1 (FIG. 7L), EFNA1 (FIG. 7M), THBS1 (FIG. 7N); and of transcription factors SMAD4 (FIG. 7P) and SMAD5 (FIG. 7Q). The coculture of exosomes showed no overexpression of skeletal development factor TFGb2 (FIG. 7D); of growth factor VEGFA (FIG. 7F); and of transcription factors SMAD2 (FIG. 7O).

In conclusion, NVD003-derived exosomes at a concentration of 100 μg/ml can potentiate the expression of osteogenic and angiogenic genes by ASCs in osteo-differentiation medium and in presence of sclerostin (100 ng/ml) in comparison to osteo-differentiation medium alone or osteo-differentiation medium+sclerostin.

2.2 Impact of NVD003-Derived Exosomes on Osteoblasts Precursors (BM-MSCs)

A model of osteoblastogenesis from human mesenchymal stem cells was used. Mesenchymal stem cells were thawed according to the recommendations of the supplier. Cells were seeded and cultured in flask in the medium recommended by the supplier for cell proliferation (RoosterBio®, KT-001).

Four days after thawing, human MSCs were detached with trypsin-EDTA and counted. The cells were seeded at 3.5·10⁴ cells per well and cultured in monolayer in 96-well plates in DMEM medium supplemented with 1% FBS for 4 days (the day of seeding is designated day-4). After 4 days of culture in DMEM medium, cells were placed in basal medium (DMEM 1% FBS+ascorbic acid (50 μg/mL) and b-glycerophosphate (10 mM), differentiation medium (positive control) (DMEM 1% FBS+ascorbic acid (50 μg/mL) and b-glycerophosphate (10 mM), dexamethasone (10-8 M) and a vitamin D3 (10-8 M)) or basal medium and NVD003-derived exosomes (the first day of treatment is “Day 0”). Medium and treatments were changed at day 4, day 7 and day 11.

As the treatment with exosomes at 75 μg/mL required an approximately 1:3 dilution for the stock solution (of a concentration of approximately 200 μg/mL), a control with PBS diluted at 1:3 in differentiation medium and a control with PBS diluted at 1:3 in basal medium were performed. All treatments and controls were carried out in duplicate.

Cells were lysed at day 7 and day 14 using Qiazol lysis reagent (Qiagen®, Hilden, Germany). For each condition, the triplicates were pooled to provide 1 cell lysate (in total, 13 cell lysates were collected at day 7 and day 14). The volume of each cell lysate was 250 μL. The cell lysates were analyzed by qRT-PCR. Total RNA was extracted from cell lysates. RNAs were purified using Rneasy mini kit (Qiagen®, Hilden, Germany) with an additional on column DNase digestion according to the manufacturer's instruction. Quality and quantity of RNA were determined using a spectrophotometer (Spectramax 190, Molecular devices®, California, USA). mRNA was concentrated using RNA Clean & Concentrator™-5 kit (ZYMO RESEARCH®, Irvine, USA). cDNA was synthesized from 0.5 μg of total RNA using RT² RNA first strand kit (Qiagen®, Hilden, Germany) for osteogenic and angiogenic genes expression profiles though a customized osteogenic and angiogenic RT² array (Qiagen®, Hilden, Germany). The ABI Quantstudio 5 system (Applied Biosystems®) and SYBR Green ROX Mastermix (Qiagen®, Hilden, Germany) were used for detection of the amplification product. Quantification was obtained according to the ΔΔCT method. The final result of each sample was normalized to the means of expression level of three housekeeping genes (ACTB, B2M and GAPDH).

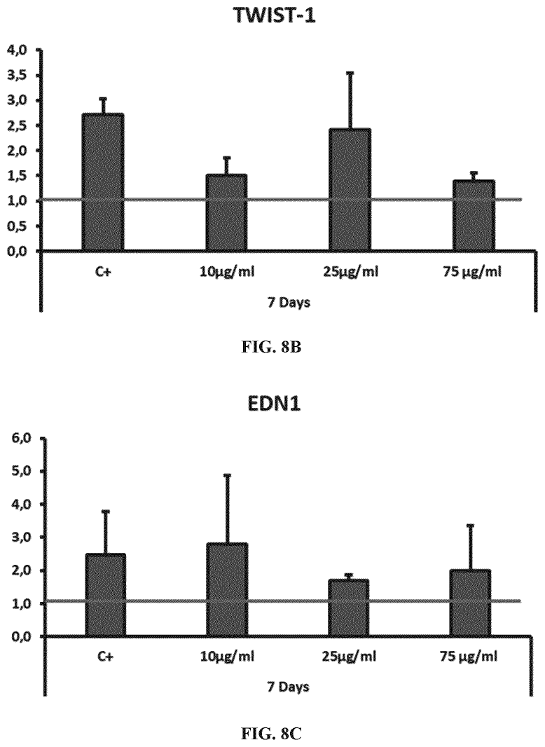

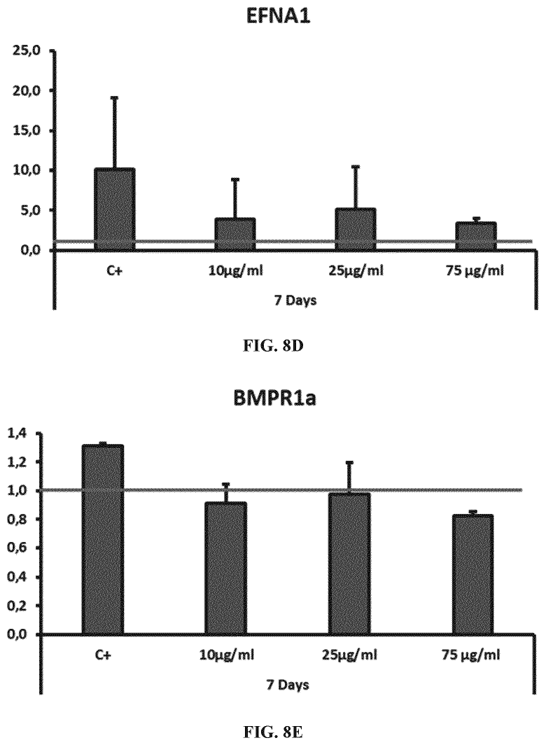

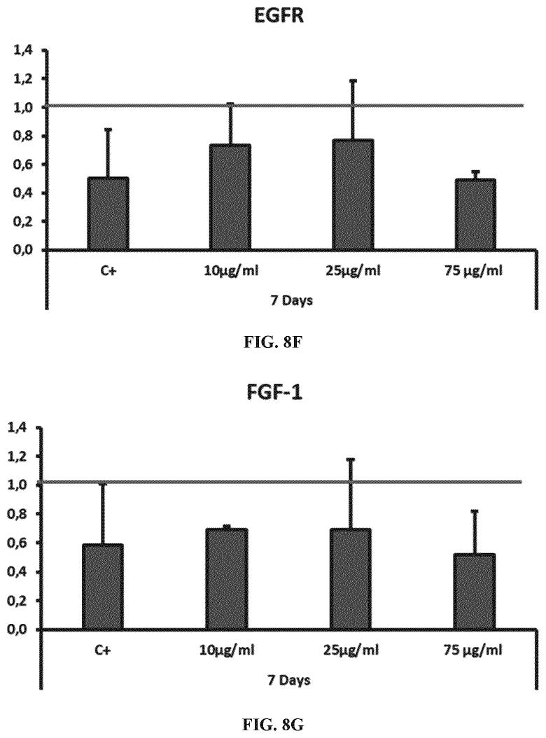

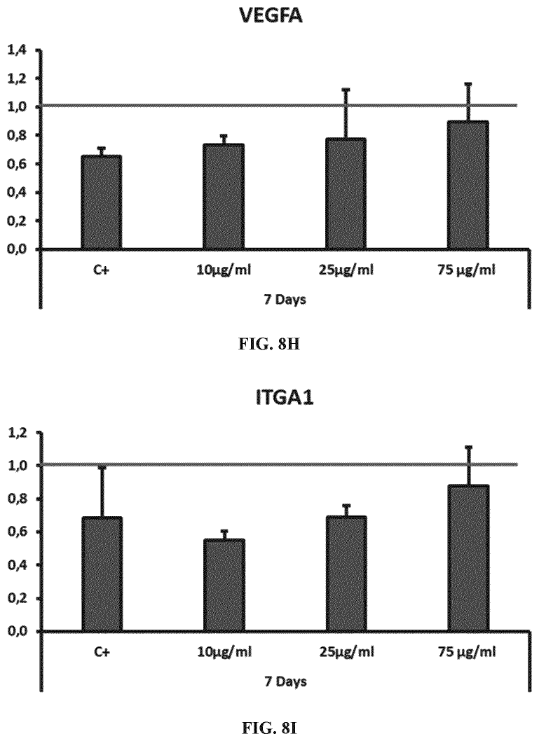

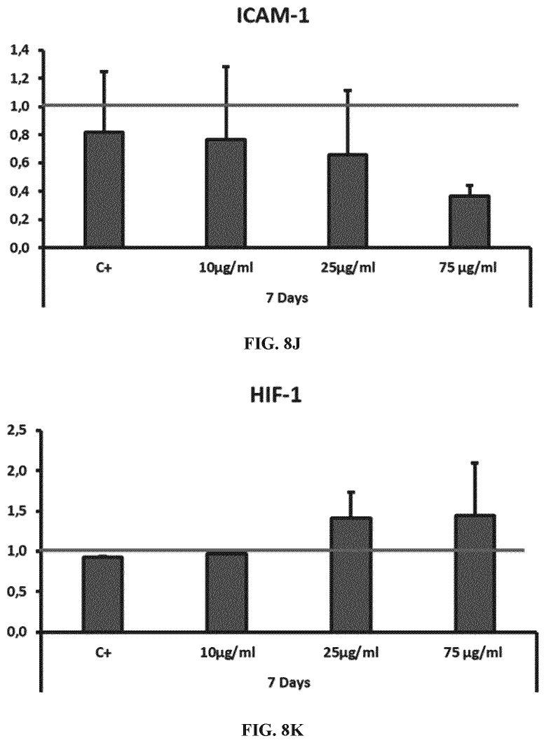

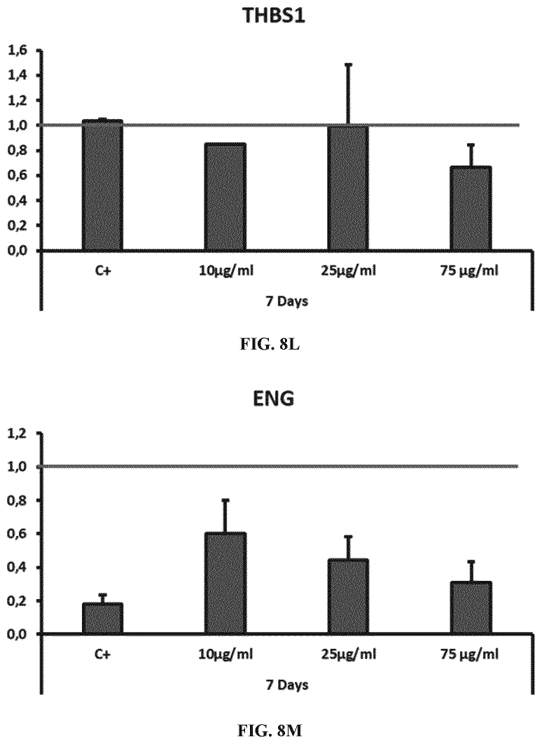

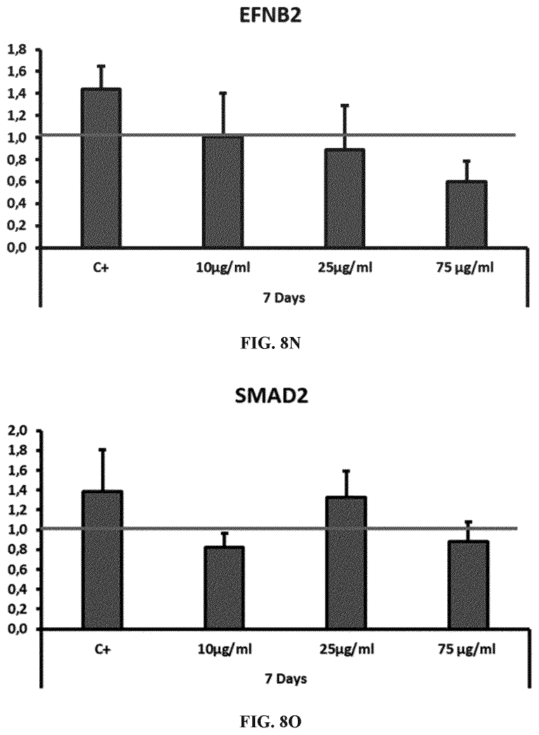

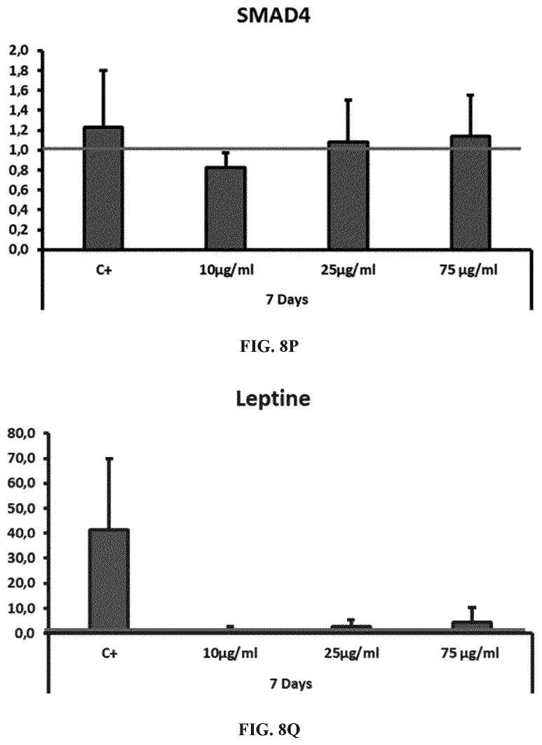

FIG. 8A-Q shows the expression of angiogenic and osteogenic genes in BM-MSCs in proliferation medium (horizontal line), BM-MSCs in osteo-differentiation medium for 7 days (C+) and BM-MSCs in proliferation medium+NVD003-derived exosomes at 10, 20 and 75 μg/ml for 7 days. Different conclusions can be made in function of the gene of interest.

Some genes are clearly induced over the basal condition (proliferation medium) as observed in the positive control (osteo-differentiation medium), such as the skeletal development factors RUNX2 (FIG. 8A) and TWIST-1 (FIG. 8B), and the angiogenic factors EDN1 (FIG. 8C) and EFNA1 (FIG. 8D). Other genes seem not to be impacted by treatment, as found in positive control, such as the skeletal development factors BMPR1a (FIG. 8E), EGFR (FIG. 8F), TGFβ1, TGFβ2, CSF1 (not shown); the growth factors FGF-1 (FIG. 8G) and, VEGFA (FIG. 8H); the cell-extracellular matrix adhesion factors, such as ITGA1 (FIG. 8I), ICAM-1 (FIG. 8J), and ITGA3 (not shown); the angiogenic factors HIF-1 (FIG. 8K), THBS1 (FIG. 8L), ENG (FIG. 8M), EFNB2 (FIG. 8N), and MMP2 (not shown); the transcription factors SMAD2 (FIG. 8O), SMAD4 (FIG. 8P), and SMADS (not shown).

Finally, Leptine expression was not induced after exosomes treatment while it was overexpressed after osteo-differentiation (see FIG. 8Q).

In conclusion, NVD003-derived exosomes treatment of BM-MSCs, at a concentration ranging from 10 μg/ml to 75 μg/ml for 7 days, shows a similar osteogenic and angiogenic expression as found in BM-MSCs in osteo-differentiation medium, except for HIF1a.

### Example 4

### In Vitro Effect of NVD002-Derived and NVD003-Derived Exosomes on Cancer Cells

1. Material and Methods

a) Cells and Exosomes

H143B human osteosarcoma cells were obtained from ATCC® (CRL-8303™), A375 human melanoma cells were obtained from ATCC® (CRL-1619™) and U87 human glioblastoma cells were obtained from ATCC® (HTB-14™). NVD002-derived and NVD003-derived exosomes are obtained as disclosed in Example 1.

b) Proliferation Assay

NVD003-derived and NVD002-derived exosomes from 3 donors were co-incubated in 96-wells plates with those three cell lines at 2.5 and 25 μg/ml for up to 72 h at 37° C., 5% CO₂. A cell viability test (using the CellTiter-Glo® Cell viability Assay from PROMEGA®) was performed after 30 minutes to 48 h of co-incubation, at minimum 5 different time points, to evaluate the proliferation of targeted cells. The CellTiter-Glo® Luminescent Cell Viability Assay from PROMEGA® is a homogeneous method to determine the number of viable cells in culture based on quantitation of the ATP present, which signals the presence of metabolically active cells). Experiments were performed in triplicate.

c) Statistical Analysis

Statistically significant differences between groups (with normal distribution) were tested by paired t-test and one-way analysis of variance with the Bonferroni post hoc test. Non-normal distributions of data were analyzed using the Kruskal-Wallis test. Statistical tests were performed with Prism GraphPad 2 (NIH). Statistical significance are as follows: *:p<0.05; **: p<0.01; ***: p<0.005; ****: p<0.0001.

2. Results

2.1 In Vitro Effect of Exosomes on Human Osteosarcoma Cells (H143B)

a) Effect of NVD002-Derived Exosomes

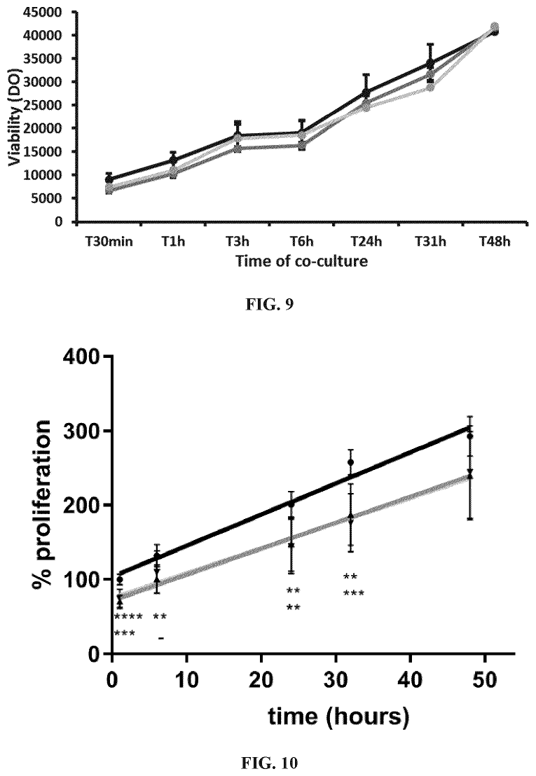

Proliferation curves of H143B cells cultured with NVD002-derived exosomes showed a slightly lower level of viability than the control cells cultured without NVD002-derived exosomes. In addition, a more marked effect was noted with the highest dose of NVD002-Exosomes exosomes (25 μg/ml vs 2.5 μg/ml) (FIG. 9).

Linear regression of the proliferation curves was calculated. Lower proliferation rates were found for cells cultured with NVD002-derived exosomes, at both 2.5 and 25 μg/ml. A significant lower viability signal was found in cells co-cultured with exosomes at 2.5 and 25 μg/ml at 1, 24 and 32 h of incubation and 2.5 μg/ml at 1, 6, 24 and 32 h of incubation (p<0.01). (FIG. 10).

Although a higher slope was found for cells cultured without NVD002-derived exosomes, it was associated with a higher viability level.

b) Effect of NVD003-Derived Exosomes

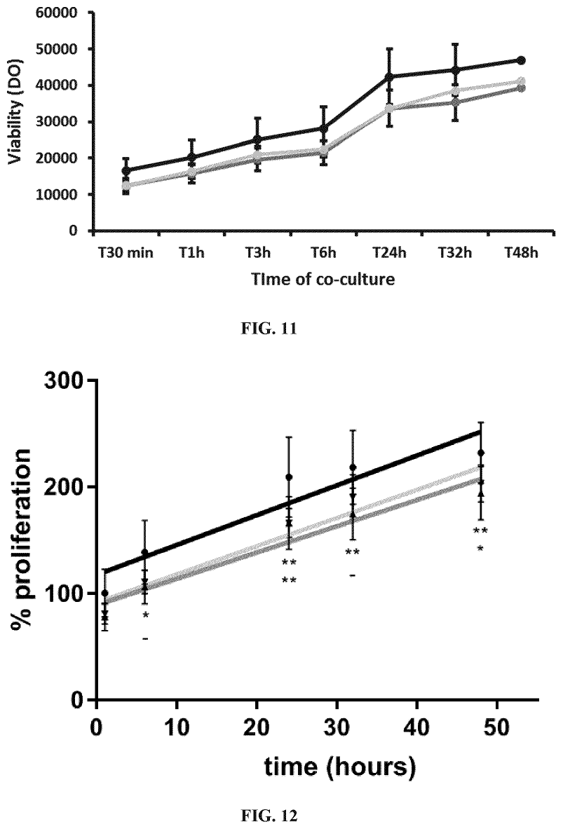

Proliferation curves of H143B cells cultured with NVD003-derived exosomes showed a slightly lower level of viability than the control cells cultured without exosomes (FIG. 11).

Linear regression of the proliferation curves was calculated. Lower proliferation rates were found for cells cultured with NVD003-derived exosomes, at both 2.5 and 25 μg/ml. A significant lower viability signal was found in cells co-cultured with NVD003-derived exosomes at 2.5 at 6, 24, 32 and 48 h of incubation and 25 μg/ml only at 24 h and 48 h of incubation p<0.01). (FIG. 12)

Although a higher slope was found for cells cultured without exosomes, it was associated with a higher viability level.

c) Conclusion

In conclusion, NVD002-derived and NVD003-derived exosomes can reduce the proliferation of human osteosarcoma cell lines in vitro. A dose-response effect was observed.

2.2 In Vitro Effect of Exosomes on Human Melanoma Cells (A375)

a) Effect of NVD002-Derived Exosomes

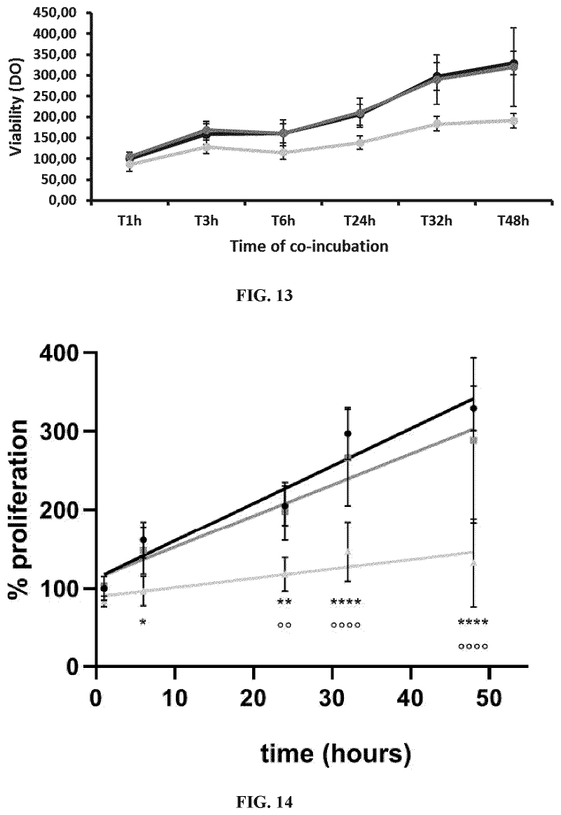

Although similar profile was found between cells cultured without exosomes and 2.5 μg/ml NVD002-derived exosomes, proliferation curves of A375 cells cultured with 25 μg/ml NVD002-derived exosomes showed a lower level of viability than the control cells cultured without exosomes (FIG. 13).

Linear regression of the proliferation curves was calculated. Lower proliferation rates were found for cells cultured with NVD002-derived exosomes, at both 2.5 and 25 μg/ml.

A significant lower viability signal was found in cells co-cultured with NVD002-derived exosomes at 25 μg/ml at 1, 24, 32 and 48 h of incubation (p<0.01). In addition, a significant lower viability signal was found at 24, 32 and 48 h in cells treated with 25 μg/ml NVD002-derived exosomes vs 2.5 μg/ml (p<0.01) (FIG. 14).

Although a higher slope was found for cells cultured without exosomes, it was associated with a higher viability level.

b) Effect of NVD003-Derived Exosomes

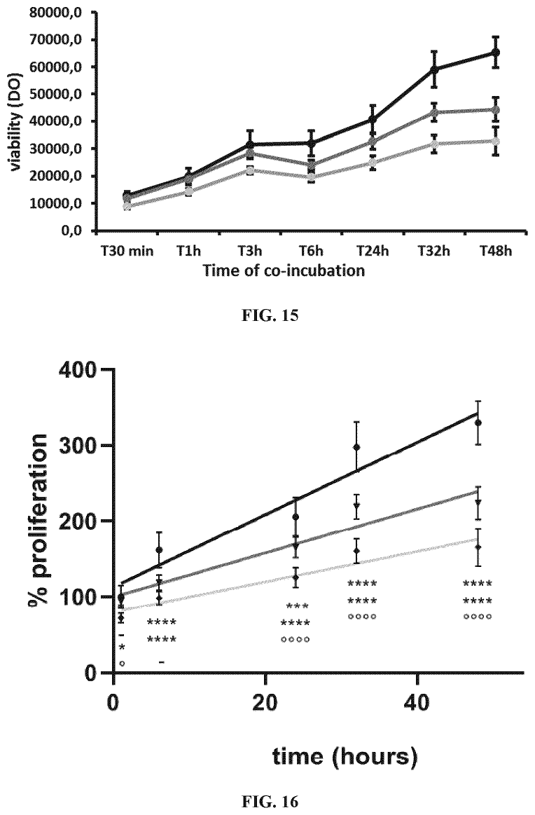

Proliferation curves of A375 cells cultured with 2.5 and 25 μg/ml NVD003-derived exosomes showed a lower level of viability than the control cells cultured without exosomes. This effect was more marked at 25 μg/ml NVD003-derived exosomes than 2.5 μg/ml (FIG. 15).

Linear regression of the proliferation curves was calculated. Lower proliferation rates were found for cells cultured with NVD003-derived exosomes, at both 2.5 and 25 μg/ml. A significant lower viability signal was found in cells co-cultured with NVD003-derived exosomes at 25 μg/ml at each time point of incubation (p<0.01). A significant lower viability signal was found in cells co-cultured with NVD003-derived exosomes at 2.5 μg/ml at 6, 24, 32 and 48 h (p<0.05). A significant lower viability signal was found in cells co-cultured with NVD003-derived exosomes at 25 μg/ml vs 2.5 μg/ml NVD003-derived exosomes at 1, 24, 32, 48 h (p<0.05) (FIG. 16).

Although a higher slope was found for cells cultured without exosomes, it was associated with a higher viability level.

c) Conclusion

In conclusion, NVD002-derived and NVD003-derived exosomes can reduce the proliferation of human melanoma cell lines in vitro. A dose-response effect was observed.

2.3 In Vitro Effect of Exosomes on Human Glioblastoma Cells (U87)

a) Effect of NVD002-Derived Exosomes

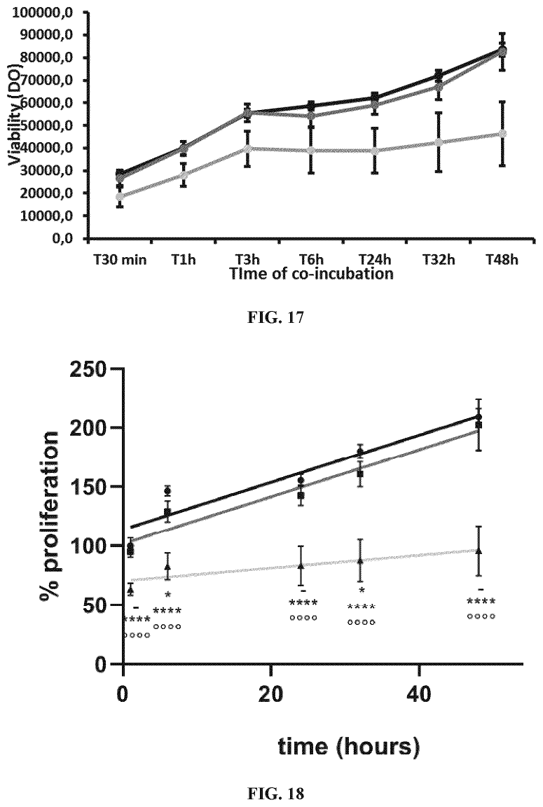

Although similar profile was found between cells cultured without exosomes and 2.5 μg/ml exosomes, proliferation curves of U87 cells cultured with 25 μg/ml exosomes showed a lower level of viability than the control cells cultured without exosomes (FIG. 17).

Linear regression of the proliferation curves was calculated. Lower proliferation rates were found for cells cultured with exosomes, at both 2.5 and 25 μg/ml, with a more marked effect at 25 than 2.5 μg/ml. A significant lower viability signal was found in cells co-cultured with exosomes at 2.5 only at 6 and 32 h (p<0.05) and 25 μg/ml at each time of incubation (p<0.0001). In addition, a significant lower viability signal was found for U87 cultured with 25 μg/ml NVD002-Exo vs 2.5 μg/ml at each tested timepoint (p<0.0001) (FIG. 18).

Although a higher slope was found for cells cultured without exosomes, it was associated with a higher viability level.

b) Effect of NVD003-Exosomes

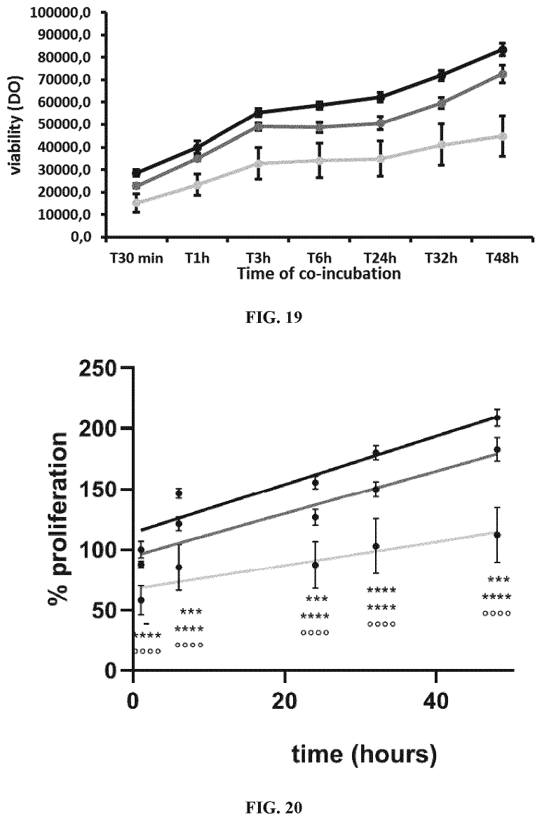

Proliferation curves of U87 cells cultured with 2.5 and 25 μg/ml exosomes showed a lower level of viability than the control cells cultured without exosomes. This effect was more marked at 25 μg/ml exosomes than 2.5 μg/ml (FIG. 19).

Linear regression of the proliferation curves was calculated. Lower proliferation rates were found for cells cultured with exosomes, at both 2.5 and 25 μg/ml. A significant lower viability signal was found in cells co-cultured with exosomes at 2.5 μg/ml at 6, 24, 32 and 48 h (p<0.0001). In addition, a significant lower viability signal was found for U87 cultured with 25 μg/ml NVD003-Exo vs 2.5 μg/ml at each tested timepoint (p<0.0001). (FIG. 20).

Although a higher slope was found for cells cultured without exosomes, it was associated with a higher viability level.

c) Conclusion

In conclusion, NVD002-derived and NVD003-derived exosomes can reduce the proliferation of human glioblastoma cell lines in vitro.

1. A pharmaceutical composition comprising (i) a therapeutically effective amount of at least three miRNAs and (ii) a pharmaceutical acceptable vehicle,
wherein said at least three miRNAs are selected in a group comprising hsa-miR-210-3p, hsa-miR-409-3p, hsa-let-7i-5p, hsa-miR-24-3p, hsa-miR-382-5p, hsa-miR-4485-3p, hsa-miR-4454, hsa-miR-619-5p, hsa-miR-3607-5p, hsa-miR-3613-3p, hsa-miR-664b-5p, hsa-miR-3687, hsa-miR-3653-5p, hsa-miR-664b-3p, and a combination thereof.

2-3. (canceled)

4. The pharmaceutical composition according to claim 1, wherein said at least three miRNAs comprise hsa-miR210-3p and/or hsa-miR-409-3p.

5. The pharmaceutical composition according to claim 1, wherein the composition is desiccated and/or sterilized.

6. A method of treatment or prevention of a tissue disorder, wherein the pharmaceutical composition according to claim 1, is administered to an animal or a human individual.

7. (canceled)

8. The method according to claim 6, wherein said tissue is selected from the group comprising bone tissue, cartilage tissue, skin tissue, muscular tissue, epithelial tissue, endothelial tissue, connective tissue, neural tissue and adipose tissue.

9. The method according to claim 6, wherein the tissue disorder is a bone disorder and/or a cartilage disorder.

10. The method according to claim 6, wherein the tissue disorder is a skin disorder.

11. The method according to claim 6, wherein the tissue disorder is selected in a group comprising aplasia cutis congenita; a burn; a cancer, including a breast cancer, a skin cancer and a bone cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer; arthritis; bone fracture; bone frailty; Caffey's disease; congenital pseudarthrosis; cranial deformation; cranial malformation; delayed union; infiltrative disorders of bone; hyperostosis; loss of bone mineral density; metabolic bone loss; osteogenesis imperfecta; osteomalacia; osteonecrosis; osteopenia; osteoporosis; Paget's disease; pseudarthrosis; sclerotic lesions; spina bifida; spondylolisthesis; spondylolysis; chondrodysplasia; costochondri tis; enchondroma; hallux rigidus; hip labral tear; osteochondritis dissecans; osteochondrodysplasia; polychondritis; and the likes.

12. The method according to claim 6, wherein the pharmaceutical composition is administered for tissue reconstruction.

13-17. (canceled)
